(19) United States
(12) Patent Application Publication
(10) Pub. No.: US 2023/0057154 A1
(43) Pub. Date: Feb. 23, 2023

(54) SOMATIC VARIANT COOCCURRENCE WITH ABNORMALLY METHYLATED FRAGMENTS

(71) Applicant: GRAIL, LLC, Menlo Park, CA (US)

(72) Inventors: Pranav Parmjit Singh, Santa Clara, CA (US); Oliver Claude Venn, San Francisco, CA (US)

(73) Assignee: GRAIL, LLC, Menlo Park, CA (US)

(21) Appl. No.: 17/817,421

(22) Filed: Aug. 4, 2022

Related U.S. Application Data
(60) Provisional application No. 63/229,797, filed on Aug. 5, 2021.

(51) Int. Cl. G16B 20/20; G16B 30/10; G16B 20/10; C12Q 1/6886; G16B 40/00; G16H 50/20; C12Q 1/6869; G16H 10/40; G06N 20/00; G06N 3/04; G06N 3/08

(52) U.S. Cl. CPC G16B 20/20; G16B 30/10; G16B 20/10; C12Q 1/6886; G16B 40/00; G16H 50/20; C12Q 1/6869; G16H 10/40; G06N 20/00; G06N 3/0454; G06N 3/084; C12Q 2600/154; C12Q 2600/112

(57) ABSTRACT

Systems and methods for identifying variant alleles as somatic or germline are provided. Reference and variant alleles for a genomic position are identified. Methylation states and sequences of nucleic acid fragment sequences that map to the genomic position are obtained from a sample of a subject. Using the sequences of nucleic acid fragment sequences, each nucleic acid fragment sequence that has the reference allele is assigned to a reference subset, and each nucleic acid fragment sequence that has the variant allele is assigned to a variant subset. One or more indications of the methylation states across the nucleic acid fragment sequences in the variant subset and an indication of the number of nucleic acid fragment sequences in the reference subset versus the variant subset are applied to a trained binary classifier. An identification of the variant allele at the genomic position as somatic or germline is obtained from the classifier.

### CROSS-REFERENCE TO RELATED APPLICATIONS

This application claims the benefit of priority from U.S. Provisional Patent Application No. 63/229,797, filed Aug. 5, 2021, which is hereby incorporated by reference herein in its entirety.

### TECHNICAL FIELD

This specification describes technologies relating to using sequencing of nucleic acid samples to determine genomic variants of a subject.

### BACKGROUND

The increasing knowledge of the molecular basis for cancer and the rapid development of next-generation sequencing techniques are advancing the study of early molecular alterations involved in cancer development and detection. Large scale sequencing technologies, such as next-generation sequencing (NGS), have afforded the opportunity to achieve sequencing at costs that are less than one U.S. dollar per million bases, and in fact costs of less than ten U.S. cents per million bases have been realized. As a result, specific genetic and epigenetic alterations associated with cancers have been found in biological samples such as plasma, serum, and urine. Such alterations can be used as diagnostic biomarkers, for instance, where methylation status and other epigenetic modifications can be correlated with the presence or classification of cancer. For example, DNA methylation plays an important role in regulating gene expression, and aberrant DNA methylation has been implicated in many disease processes, including certain cancer conditions.

Specific patterns of differentially methylated regions and/or allele specific methylation patterns obtained using methylation sequencing may therefore be useful as molecular markers for non-invasive diagnostics using circulating cell-free DNA (cfDNA). Found in serum, plasma, urine, and other body fluids, cfDNA provides a circulating picture of disease in a biological subject, including, for example, specific tumor-related alterations such as mutations, methylation, and copy number variations. The analysis of cfDNA in liquid biopsies obtained from subjects with cancer conditions presents an attractive opportunity for non-invasive methods of screening for a variety of cancers.

In addition, approaches using deep learning to model and infer complex biological patterns and non-linearities across the genome can be used in the development of clinical and analytical tools for cancer. For example, deep learning strategies using nucleic acid sequences can be used for various classification, regression, inference and clustering cancer objectives, including Neu-Somatic, DeepVariant, methylation state predictions, and denoising histone. Deep learning approaches aim, in part, to address the rapid and substantial increases in the amount, size, and complexity of sequencing datasets accompanying new, large-scale sequencing technologies. For example, the assembly and organization of large quantities of high-fidelity nucleic acid sequences into complete genomes, and the analysis and identification of potential diagnostic indicators therein, are computationally challenging tasks.

Along with the promise and possibilities of applying deep learning to nucleic acid sequencing data, there are numerous caveats and dangers to avoid, including large class imbalance due to low prevalence of cancer in general populations, insufficient number of training examples relative to number of learned parameters, and susceptibility to overfit on biological or process related noise, among others. Similarly, although cancer prediction can be approached using numerous modeling techniques (e.g., clustering, outlier, denoising or classification) using various architectures such as auto-encoder, recurrent, transformer, wide and deep, embeddings or convolutional networks, optimally framing the problem for accurate prediction and minimizing the data imbalances, noise, overfitting and sparsity are pivotal challenges that need careful consideration.

For example, sample quality and/or purity in training datasets may vary due to the inclusion of mixed sample types, resulting in poor classifier performance (e.g., when using cfDNA from liquid biopsies, which can be derived from multiple cell and/or tissue origins). Obtaining a sufficient number of high-quality training samples that can be confidently annotated with the conditions of interest (e.g., cancer, non-cancer and/or cancer subtype) for accurate training of a classifier therefore presents a challenge.

Additionally, the identification of nucleic acid fragments with tumor-specific variants in cancer patients remains challenging due to the high proportion of nucleic acid fragments that originate from healthy tissue compared to those that originate from tumor tissue. Such problems are encountered particularly when using cfDNA fragments obtained from liquid biopsy samples but can also arise due to clonal heterogeneity in solid tumors.

Given the above, there is a need in the art for methods of analyzing genetic information from nucleic acid sequencing data, including data obtained from cfDNA.

### SUMMARY

The present disclosure addresses the shortcomings identified in the background by providing robust techniques for identifying genomic variants as somatic or germline from biological samples obtained from a subject using nucleic acid data. The combination of methylation data with whole genome and/or targeted genome sequencing data provides additional diagnostic power beyond previous screening methods.

Technical solutions (e.g., computing systems, methods, and non-transitory computer-readable storage mediums) for addressing the above-identified problems with analyzing datasets are provided in the present disclosure.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

One aspect of the present disclosure provides a method of identifying a variant allele at a genomic position in a test subject as somatic or germline. The method comprises obtaining an identification of a reference allele at the genomic position, obtaining an identification of the variant allele at the genomic position, and obtaining a methylation state and a respective sequence of each nucleic acid fragment sequence in a respective plurality of nucleic acid fragment sequences in a sequencing dataset (e.g., comprising at least 10\hat{ }6 nucleic acid fragment sequences) derived from a biological sample obtained from the test subject that map onto the genomic position.

The identification of the reference allele at the genomic position and the respective sequence of each nucleic acid fragment sequence in the respective plurality of nucleic acid fragment sequences are used to assign each nucleic acid fragment sequence in the respective plurality of nucleic acid fragment sequences that has the reference allele, at the genomic position, to a reference subset. Additionally, the identification of the variant allele at the genomic position and the respective sequence of each nucleic acid fragment sequence in the respective plurality of nucleic acid fragment sequences are used to assign each nucleic acid fragment sequence in the respective plurality of nucleic acid fragment sequences that has the variant allele, at the genomic position, to a variant subset.

At least (i) one or more indications of methylation state across the methylation state of each nucleic acid fragment sequence in the variant subset and (ii) an indication of a number of nucleic acid fragment sequences in the reference subset versus a number of nucleic acid fragment sequences in the variant subset are applied to a trained binary classifier (e.g., comprising at least 10 parameters), thus obtaining from the trained binary classifier an identification of the variant allele at the genomic position in the test subject as somatic or germline.

In some embodiments, the method further comprises inputting a reference genome into a computer system comprising a processor coupled to a non-transitory memory, and using the computer system to determine that each respective nucleic acid fragment sequence in the respective plurality of nucleic acid fragment sequences maps to the genomic position by aligning the respective nucleic acid fragment sequence to the reference genome.

In some embodiments, a first nucleic acid fragment sequence in the respective plurality of nucleic acid fragment sequences has a plurality of CpG sites, the first nucleic acid fragment sequence has a corresponding methylation pattern across the plurality of CpG sites, the methylation state of the first nucleic acid fragment sequence is a p-value, and the method further comprises determining the p-value of the first nucleic acid fragment sequence, at least in part, by comparison of the corresponding methylation pattern of the first nucleic acid fragment sequence to a corresponding distribution of methylation patterns of those nucleic acid fragment sequences in a healthy noncancer cohort dataset that each have the respective plurality of CpG sites.

In some embodiments, when the variant allele at the genomic position is determined by the trained binary classifier to be germline, the method further comprises using the variant allele in the test subject to determining a cancer risk of the test subject. In some embodiments, when the variant allele at the genomic position is determined by the trained binary classifier to be germline, the method further comprises using the variant allele in the test subject to predict an ethnicity of the subject. In some embodiments, when the variant allele at the genomic position is determined by the trained binary classifier to be somatic, the method further comprises using the variant allele in the test subject to determine a tumor fraction of the subject.

In some embodiments, the applying, to the trained binary classifier, further applies one or more CpG site indications across the variant subset.

In some embodiments, the applying, to the trained binary classifier, further applies one or more indications of methylation state across the reference subset.

In some embodiments, the applying, to the trained binary classifier, further applies one or more CpG site indications across the reference subset.

In some embodiments, the obtaining the identification of the variant allele at the genomic position comprises obtaining, for the genomic position, a strand-specific base count set, where the strand-specific base count set comprises a strand-specific count for each base in the set of bases (e.g., A, C, T, G) at the genomic position, in a forward direction and a reverse direction, that is acquired by determining (i) a strand orientation and (ii) an identity of a respective base at the genomic position in each respective nucleic acid fragment sequence in the respective plurality of nucleic acid fragment sequences, and where bases at the genomic position in the respective plurality of nucleic acid fragment sequences whose identity can be affected by conversion of methylated or unmethylated cytosine do not contribute to the strand-specific base count set. A respective forward strand conditional probability and a respective reverse strand conditional probability are computed for each respective candidate genotype in the set of candidate genotypes for the genomic position using the strand-specific base count set and a sequencing error estimate, thus computing a plurality of forward strand conditional probabilities and a plurality of reverse strand conditional probabilities. A plurality of likelihoods are computed, each respective likelihood in the plurality of likelihoods for a respective candidate genotype in the set of candidate genotypes, where the computing uses a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype. The plurality of likelihoods is used to identify the variant allele at the genomic position, thus obtaining the identification of the variant allele at the genomic position.

In some embodiments, the method further comprises repeating the method for each genomic position in a plurality of genomic positions, thus identifying a plurality of variants for the test subject, and for each respective variant in the plurality of variants, identifying whether the respective variant is somatic or germline.

Another aspect of the present disclosure provides a method of training a classifier (e.g., comprising at least 10 parameters) to identify a variant allele at a genomic position in a test subject as somatic or germline. The method comprises obtaining an identification of a reference allele at the genomic position and performing a procedure for each respective genomic position in a plurality of genomic positions, for each respective subject in a plurality of subjects.

The procedure comprises i) obtaining an orthogonal call for the variant allele at the respective genomic position as one of somatic or germline for the respective subject, ii) obtaining an identification of the variant allele at the respective genomic position for the respective subject, iii) obtaining a methylation state and a respective sequence of each nucleic acid fragment sequence in a respective plurality of nucleic acid fragment sequences in a sequencing dataset (e.g., comprising at least 10\hat{ }6 nucleic acid fragment sequences) derived from a biological sample obtained from the respective subject that map onto the respective genomic position, iv) using (a) the identification of the reference allele at the respective genomic position and (b) the respective sequence of each nucleic acid fragment sequence in the respective plurality of nucleic acid fragment sequences to assign each nucleic acid fragment sequence in the respective plurality of nucleic acid fragment sequences that has the reference allele, at the respective genomic position, to a reference subset, and v) using (a) the identification of the variant allele at the respective genomic position and (b) the respective sequence of each nucleic acid fragment sequence in the respective plurality of nucleic acid fragment sequences to assign each nucleic acid fragment sequence in the respective plurality of nucleic acid fragment sequences that has the variant allele, at the respective genomic position, to a variant subset.

For each respective subject in the plurality of subjects, for each respective genomic position in the plurality of genomic positions, at least (i) one or more indications of methylation state across the methylation state of each nucleic acid fragment sequence in the variant subset for the respective subject for the respective genomic position, (ii) an indication of a number of nucleic acid fragment sequences in the reference subset versus a number of nucleic acid fragment sequences in the variant subset for the respective subject for the respective genomic position, and (iii) the orthogonal call for the variant allele at the respective genomic position as one of somatic or germline for the respective subject are used to train the classifier to identify a variant allele at a genomic position in a test subject as somatic or germline.

Another aspect of the present disclosure provides a computing system, comprising one or more processors and memory storing one or more programs to be executed by the one or more processor, the one or more programs comprising instructions for performing any of the methods disclosed above alone or in combination.

Still another aspect of the present disclosure provides a non-transitory computer readable storage medium storing one or more programs configured for execution by a computer, where the one or more programs comprise instructions for performing any of the methods disclosed above alone or in combination.

Various embodiments of systems, methods, and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 illustrates an example block diagram illustrating a computing device in accordance with some embodiments of the present disclosure.

FIGS. 2A and 2B collectively illustrate an example flowchart of a method of identifying a variant allele at a genomic position in a test subject as somatic or germline, in which dashed boxes represent optional steps, in accordance with some embodiments of the present disclosure.

FIG. 3 illustrates an example flowchart of a method of calling a variant allele, in accordance with some embodiments of the present disclosure.

FIGS. 4A and 4B illustrate analysis of correlation between methylation patterns and somatic variants, in accordance with some embodiments of the present disclosure.

FIGS. 5A and 5B illustrate example performance measures for a method in accordance with some embodiments of the present disclosure.

FIGS. 6A and 6B illustrate example performance measures for a method in accordance with some embodiments of the present disclosure.

FIG. 7 illustrates a flowchart of a method for preparing a nucleic acid sample for sequencing, in accordance with some embodiments of the present disclosure.

FIG. 8 is a graphical representation of a process for obtaining sequence reads, in accordance with some embodiments of the present disclosure.

FIG. 9 illustrates an example flowchart of a method for obtaining methylation information in a subject, in accordance with some embodiments of the present disclosure.

FIGS. 10A and 10B illustrate example performance measures for a method in accordance with some embodiments of the present disclosure.

FIGS. 11A and 11B illustrate example performance measures for a method in accordance with some embodiments of the present disclosure.

### DETAILED DESCRIPTION

### Introduction

As described above, conventional methods for analyzing nucleic acid sequencing data may not provide accurate determination of cancer-associated biomarkers. For example, though recent developments in next-generation sequencing technologies and machine learning have led to advances in the analysis of sequencing data, accurate determination of genetic variants using cfDNA is hampered by the presence of nucleic acid molecules derived from other tissues such as healthy tissue. Conventional methods may include obtaining and sequencing a patient-matched normal (e.g., healthy) control sample such as white blood cells or tissue biopsies, and performing a comparative analysis to determine which mutations observed in the liquid biopsy sample are likely to originate from a tumor and which originate from the normal control.

In the absence of a matched normal control, it may be difficult to determine whether a genomic alteration is a germline variant or a somatic variant, particularly for uncommon or unannotated variants. However, unlike liquid biopsy samples, matched normal controls may not be routinely obtained in clinical settings. For instance, as described herein, use of bodily fluids advantageously facilitates clinical applications because of the ease of collection, as these fluids are obtainable by non-invasive or minimally invasive methodologies. This may be in contrast to methods that rely upon solid tissue samples, such as biopsies, which often use invasive surgical procedures. Thus, improved methods described herein may comprise analyzing nucleic acid sequencing data to accurately identify and classify genetic variants, such as tumor-specific variants, in cfDNA. In particular, improved methods may comprise identifying variant alleles as somatic or germline.

Advantageously, the present disclosure provides methods and systems that do provide accurate determination of variant alleles as somatic or germline. For example, in some embodiments, the methods and systems described herein include using nucleic acid sequencing and methylation sequencing of nucleic acid fragments in a liquid biopsy sample to obtain a plurality of features for input into a binary classifier trained to identify a variant allele in a subject as somatic or germline. Each nucleic acid fragment that maps to the genomic position of the variant allele may be binned into a variant subset if the corresponding sequence read (e.g., obtained from the nucleic acid sequencing) has support for the variant allele, or is binned into a reference subset if the corresponding sequence read has support for the reference allele. The features used as input into the classifier may include at least a count of nucleic acid fragments in the variant subset, a count of nucleic acid fragments in the reference subset, and one or more distribution statistics for p-values calculated across the methylation vectors (e.g., obtained from the methylation sequencing) corresponding to the nucleic acid fragments in the variant subset and the reference subset, respectively. In some embodiments, the features further include a count of CpG sites in the nucleic acid fragments assigned to the variant subset and a count of CpG sites in the nucleic acid fragments assigned to the reference subset. This may result in an output, from the trained binary classifier, that identifies whether the variant allele at the genomic position in the subject is somatic or germline.

The accurate identification of variants as somatic or germline may provide advantages to such clinical applications as diagnosing cancer, determining stage of cancer, monitoring cancer progression, determining prognosis, prescribing or administering treatments, matching or recommending enrollment in clinical trials, monitoring the development of additional complications or risks over time, and evaluating efficacy of treatment, among others.

For example, somatic variants reflect genetic mutations that are accumulated over a subject's lifetime through a mutagenic process (e.g., smoking, drinking, etc.) and are more closely connected with the development of cancer. Potential therapeutic uses of somatic variant identification may include the increased ability of physicians to interpret cancer types and select the most effective treatment option. Thus, the accurate identification of genetic variants as somatic or germline can impact the ability of healthcare providers to determine appropriate treatment recommendations for patients. In addition to cancer risk, monitoring, and treatment, identification of somatic variants using the methods described herein can also be used for tumor fraction estimation (e.g., to confirm or to supplement tumor mutational burden calculations obtained using matched normal control samples). Furthermore, somatic variants can be indicative for other disease types, including clonal hematopoiesis of indeterminate potential (CHIP), cardiovascular risk, nonalcoholic fatty liver disease (NAFLD or NASH), and other disease states.

In contrast, germline variants may not be involved with the development of cancer and as such typically provide less information than somatic variants in terms of detecting and/or identifying cancer. Nevertheless, germline variants can provide information on prior cancer risk, either through the identification of annotated cancer-associated germline variants (e.g., BRCA) or through the calculation of polygenic risk scores (PRS) using genetic information. Additionally, the accurate identification of germline variants can be used in analytical processing such as in the enrichment of somatic variants in datasets, or for other applications such as ethnicity prediction.

Advantageously, the presently disclosed methods can overcome the abovementioned difficulties of identifying somatic variants in the absence of normal (e.g., healthy) controls by using methylation patterns to improve the quality of variant calling in nucleic acid sequencing data. The presently disclosed methods can leverage the potential for co-occurrence between abnormal methylation signals with enrichment of somatic variants, in combination with machine learning algorithms, to improve upon prior art methods of variant classification using nucleic acid sequencing alone.

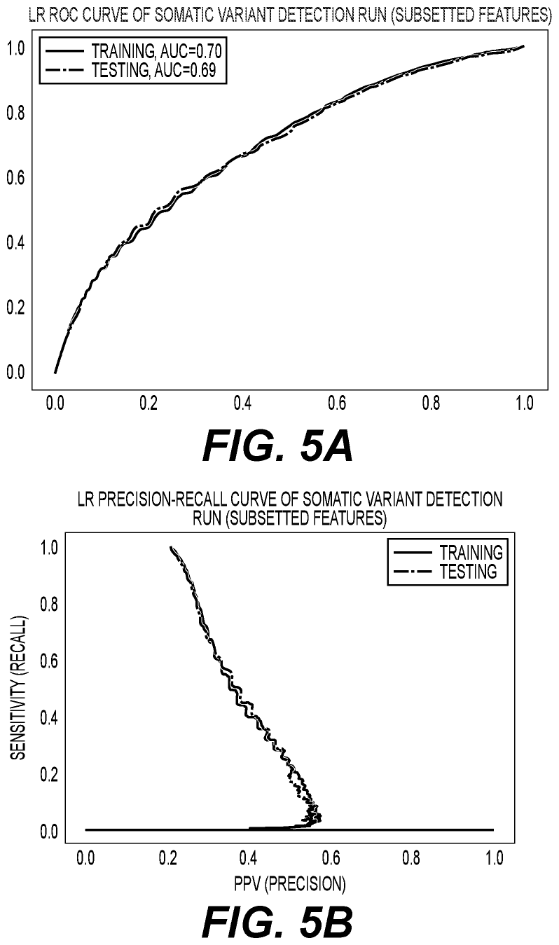

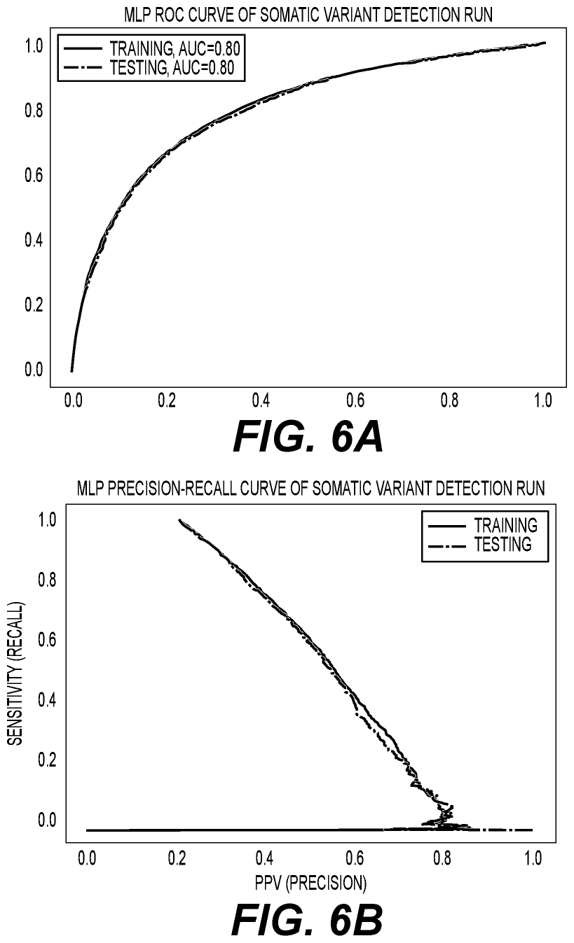

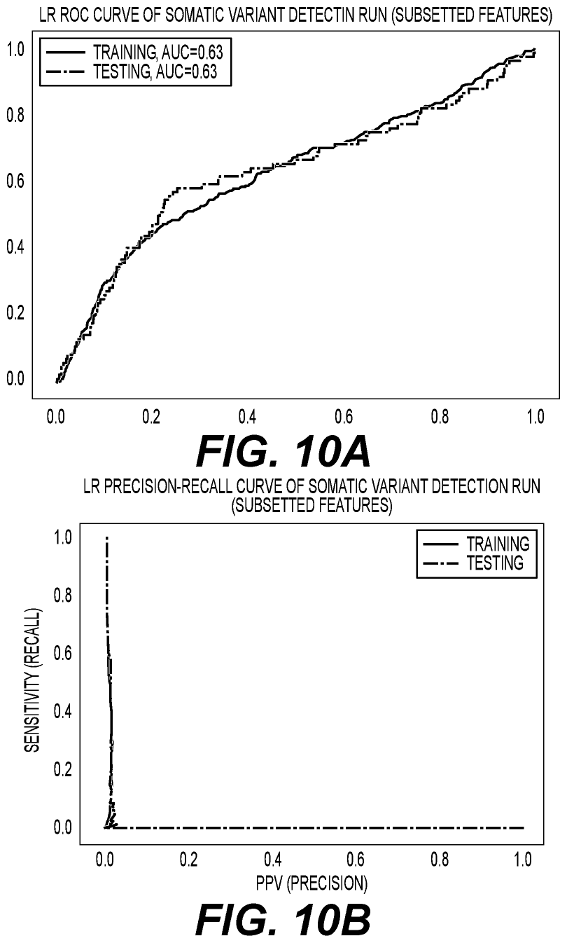

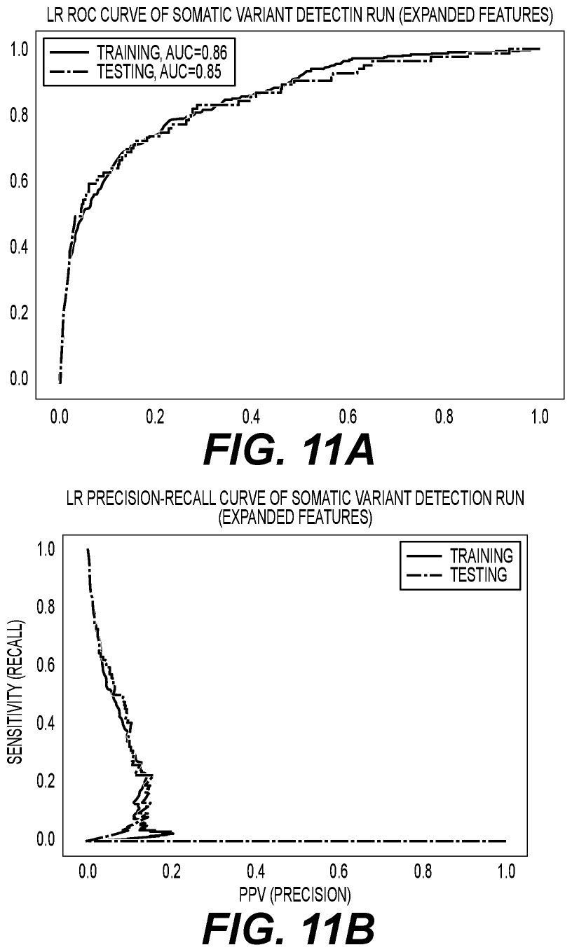

Specifically, the addition of p-value and CpG distribution statistics based on methylation sequencing of nucleic acid fragments to the input vector for a trained binary classifier may result in improved performance in the classifier, compared to baseline inputs containing reference and variant fragment counts obtained using nucleic acid sequence reads. For example, as reported in Example 6, when methylation fragment p-values and CpG counts were added to a baseline input of reference and variant fragment counts, the performance of logistic regression and neural network classifiers improved with respect to area under curve (AUC), positive predictive value (precision), and sensitivity (recall). Improvements were observed both when using tissue-derived sequencing datasets, as shown in FIGS. 5A, 5B, 6A, and 6B, and when using cfDNA-derived sequencing datasets, as shown in FIGS. 10A, 10B, 11A, and 11B.

The methods and systems described thus can improve methods for assigning and/or administering treatment because of the improved accuracy of variant identification as somatic or germline.

Additional Benefits.

The identification of genomic alterations in a patient's cancer genome can be a difficult and computationally demanding problem. For instance, the determination of various prognostic metrics useful for clinical action, including the identification and classification of variant alleles, uses analysis of hundreds of millions to billions of sequenced nucleic acid bases. An example of a typical bioinformatics pipeline established for this purpose can include at least five stages of analysis: assessment of the quality of raw next generation sequencing data, generation of collapsed nucleic acid fragment sequences and alignment of such sequences to a reference genome, detection of structural variants in the aligned sequence data, annotation of identified variants, and visualization of the data.

Furthermore, the presently disclosed method can add such processes as performing methylation sequencing, correlating each methylation fragment sequence to the respective nucleic acid fragment and its corresponding nucleic acid sequence, binning the plurality of nucleic acid fragments at each variant position, faceting nucleic acid fragments based on reference or alternate support, determining, for the plurality of fragments binned at each variant position, a plurality of features (including but not limited to reference fragment count, alternate fragment count, methylation state p-value distribution statistics, and/or CpG site count distribution statistics), and generating feature vectors for input to a binary classifier. In some aspects of the present disclosure, the method can further comprise training a binary classifier to identify variants as somatic or germline, based on a training dataset comprising a plurality of training subjects. Each one of these steps can be computationally taxing in its own right.

For instance, the overall temporal and spatial computation complexity of simple global and local pairwise sequence alignment algorithms can be quadratic in nature (i.e., second order problems), that increase rapidly as a function of the size of the nucleic acid sequences (n and m) being compared. Specifically, the temporal and spatial complexities of these sequence alignment algorithms can be estimated as O(mn), where O is the upper bound on the asymptotic growth rate of the algorithm, n is the number of bases in the first nucleic acid sequence, and m is the number of bases in the second nucleic acid sequence. Given that the human genome contains more than 3 billion bases, these alignment algorithms can be extremely computationally taxing, especially when used to analyze next generation sequencing (NGS) data, which can generate more than 3 billion sequence reads per reaction.

This can be particularly true when performed in the context of a liquid biopsy assay, because liquid biological samples can contain a complex mixture of short DNA fragments originating from many different germline (e.g., healthy) and diseased (e.g., cancerous) tissues. Thus, the cellular origins of the sequence reads can be unknown, and the sequence signals originating from cancerous cells, which may constitute multiple sub-clonal populations, may be computationally deconvoluted from signals originating from germline and hematopoietic origins, in order to provide relevant information about the subject's cancer. Thus, in addition to the computationally taxing processes used to align sequence reads to a human genome, there can be a computation problem of determining whether a particular abnormal signal, e.g., one or more sequence reads corresponding to a genomic alteration, (i) is not an artifact, and (ii) originated from a cancerous source in the subject. This can be increasingly difficult during the early stages of cancer—when treatment is presumably most effective—when small amounts of circulating tumor DNA (ctDNA) are diluted by germline and hematopoietic DNA.

Advantageously, the present disclosure provides various systems and methods that improve the computational elucidation of genomic alterations (e.g., somatic or germline variants) from cfDNA in a subject. The methods and systems described herein can solve a problem in the computing art, e.g., by improving the accuracy of identification of variants as somatic or germline. As detailed above, the classification of variants can comprise a plurality of processes that can be performed as a bioinformatics pipeline, each of which utilize large-scale sequencing datasets (e.g., at least 1×10⁶ sequence reads), accompanied by temporal and spatial computation complexity that increases with the size of the sequencing dataset at a quadratic rate. Large requirements on computational power, including processing time and processing space, can reduce the efficiency of computer-implemented methods. Considering these constraints, the improvement of such a process can provide a solution to a computing art, by providing more efficient and accurate methods for variant identification.

Further advantageously, the present disclosure provides various systems and methods that improve the computational elucidation of genomic alterations (e.g., somatic or germline variants) from cfDNA in a subject by improving the training and use of a model for more accurate variant identification. The complexity of a machine learning model can include time complexity (running time, or the measure of the speed of an algorithm for a given input size n), space complexity (space requirements, or the amount of computing power or memory needed to execute an algorithm for a given input size n), or both. Complexity (and subsequent computational burden) can apply to both training of and prediction by a given model.

In some instances, computational complexity can be affected by implementation, incorporation of additional algorithms or cross-validation methods, and/or one or more parameters (e.g., weights and/or hyperparameters). Nevertheless, computational complexity can generally be expressed as a function of input size n, where input data is the number of instances (e.g., the number of training samples), dimensions p (e.g., the number of features), the number of trees nₜᵣₑₑₛ (e.g., for methods based on trees), the number of support vectors nₛᵥ (e.g., for methods based on support vectors), the number of neighbors k (e.g., fork nearest neighbor algorithms), the number of classes c, and/or the number of neurons nᵢ at a layer i (e.g., for neural networks). With respect to input size n, then, an approximation of computational complexity (e.g., in Big O notation) denotes how running time and/or space requirements increase as input size increases. Functions can increase in complexity at slower or faster rates relative to an increase in input size. Various approximations of computational complexity include but are not limited to constant (e.g., O(1)), logarithmic (e.g., O(log n)), linear (e.g., O(n)), loglinear (e.g., O(n log n)), quadratic (e.g., O(n²)), polynomial (e.g., O(n^{c}), exponential (e.g., O(c)), and/or factorial (e.g., O(n!)). In some instances, simpler functions are accompanied by lower levels of computational complexity as input sizes increase, as in the case of constant functions, whereas more complex functions such as factorial functions can exhibit substantial increases in complexity in response to slight increases in input size.

Computational complexity of machine learning models can similarly be represented by functions (e.g., in Big O notation), and complexity may vary depending on the type of model, the size of one or more inputs or dimensions, usage (e.g., training and/or prediction), and/or whether time or space complexity is being assessed. For example, complexity in decision tree algorithms is approximated as O(n²p) for training and O(p) for predictions, while complexity in linear regression algorithms is approximated as O(p²n+p³) for training and O(p) for predictions. For random forest algorithms, training complexity can be approximated as O(n²pnₜᵣₑₑₛ) and prediction complexity is approximated as O(pnₜᵣₑₑₛ). For gradient boosting algorithms, complexity can be approximated as O(npnₜᵣₑₑₛ) for training and O(pnₜᵣₑₑₛ) for predictions. For kernel support vector machines, complexity can be approximated as O(n²p+n³) for training and O(nₛᵥp) for predictions. For naïve Bayes algorithms, complexity can be represented as O(np) for training and O(p) for predictions, and for neural networks, complexity can be approximated as O(pn₁+n₁n₂+ . . . ) for predictions. Complexity in K nearest neighbors algorithms can be approximated as O(knp) for time and O(np) for space. For logistic regression algorithms, complexity can be approximated as O(np) for time and O(p) for space. For logistic regression algorithms, complexity can be approximated as O(np) for time and O(p) for space.

As described above, for machine learning models, computational complexity can dictate the scalability and therefore the overall effectiveness and usability of a model (e.g., a classifier) for increasing input, feature, and/or class sizes, as well as for variations in model architecture. In the context of large-scale sequencing technologies, the computational complexity of functions performed on sequencing datasets (e.g., nucleic acid sequencing data and methylation sequencing data obtained from cfDNA samples) may strain the capabilities of many existing systems. In addition, as the number of input features (e.g., reference and alternate counts, p-value distribution statistics (e.g., mean, min, max, median, standard deviation), and/or CpG site distribution statistics (e.g., mean, min, max, median, standard deviation), stratified for reference and alternate subsets) and/or the number of instances (e.g., training subjects, test subjects, number of variant alleles, and/or number of genomic positions) increases with expanding downstream applications and possibilities, the computational complexity of any given classification model can quickly overwhelm the time and space capacities provided by the specifications of a respective system.

Generally (and as defined herein), parameters (e.g., weights and/or hyperparameters) are coefficients that modulate one or more inputs, outputs, or functions in a model. For instance, a value of a parameter can be used to upweight or down-weight the influence of an input to a model, such as a feature. Thus, features can be associated with parameters, such as in a logistic regression, SVM, or naïve Bayes model. A value of a parameter can, alternately or additionally, be used to upweight or down-weight the influence of a node in a neural network (e.g., where the node comprises one or more activation functions that define the transformation of an input to an output), a class, or an instance (e.g., of a sample). Assignment of parameters to specific inputs, outputs, functions, or features can be any one paradigm for a given model but can be used in any suitable model architecture for optimal performance. Nevertheless, reference to the coefficients associated with the inputs, outputs, functions, or features of a model can similarly be used as an indicator of the number, performance, or optimization of the same, such as in the context of the computational complexity of machine learning algorithms.

Thus, a machine learning model with a minimum input size (e.g., at least 1×10⁶ sequence reads) and/or a minimum number of parameters (e.g., at least 10, at least 100, or at least 1000 parameters) can refer to a corresponding number of associated inputs, outputs, functions, or features in the model. The computational complexity of such a model can be proportionally increased such that use of the model for the presently disclosed method (e.g., the identification of somatic or germline variants from cfDNA in a subject) cannot be mentally performed, and the method can be inherently a computational problem.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

As used herein, the term “about” or “approximately” means within an acceptable error range for the particular value as determined by one of ordinary skill in the art, which depends in part on how the value is measured or determined, e.g., the limitations of the measurement system. For example, in some embodiments “about” mean within 1 or more than 1 standard deviation, per the practice in the art. In some embodiments, “about” means a range of ±20%, ±10%, ±5%, or ±1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value can be assumed. The term “about” can have the meaning as commonly understood by one of ordinary skill in the art. In some embodiments, the term “about” refers to ±10%. In some embodiments, the term “about” refers to ±5%.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. For example, as used herein, the term “between” used in a range is intended to include the recited endpoints. For example, a number “between X and Y” can be X, Y, or any value from X to Y.

As used herein, the term “allele” refers to a particular sequence of one or more nucleotides at a genomic position. For haploid organisms, a subject generally has one allele at every genomic position. For diploid organisms, a subject generally has two alleles at every genomic position.

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay can be used to detect any of the properties of nucleic acids mentioned herein. Properties of nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

As used herein, the term “biological sample” or “sample” refers to any sample taken from a subject (i.e., any type of organism, not just humans), which can reflect a biological state associated with the subject. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample and/or include cell-free DNA. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample).

As used herein, the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: a degree of cellular differentiation including morphology and functionality, rate of growth, local invasion and metastasis. A “benign” tumor can be well-differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used interchangeably herein, the terms “cancer load,” “tumor load,” “cancer burden,” “tumor burden,” or “tumor fraction” refer to a concentration or presence of tumor-derived nucleic acids in a test sample. As such, the terms “cancer load,” “tumor load,” “cancer burden,” “tumor burden,” and “tumor fraction” are non-limiting examples of a cell source fraction in a biological sample. In some embodiments, tumor fraction is a specific version of cell source fraction.

As disclosed herein, the terms “cell-free nucleic acid,” “cell-free DNA,” and “cfDNA” interchangeably refer to nucleic acid fragments that circulate in a subject's body (e.g., in a bodily fluid such as the bloodstream) and originate from one or more healthy cells and/or from one or more cancer cells. Cell-free DNA may be recovered from bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids are used interchangeably with circulating nucleic acids. Examples of cell-free nucleic acids include but are not limited to RNA, mitochondrial DNA, or genomic DNA.

As disclosed herein, the term “circulating tumor DNA” or “ctDNA” refers to nucleic acid fragments that originate from aberrant tissue, such as the cells of a tumor or other types of cancer, which may be released into a subject's bloodstream as result of biological processes such as apoptosis or necrosis of dying cells or actively released by viable tumor cells.

As used herein, the term “classification” refers to any number(s) or other characters(s) that are associated with a particular property of a sample. For example, a “+” symbol (or the word “positive”) can signify that a sample is classified as having deletions or amplifications. In another example, the term “classification” refers to an amount of tumor tissue in the subject and/or sample, a size of the tumor in the subject and/or sample, a stage of the tumor in the subject, a tumor load in the subject and/or sample, and presence of tumor metastasis in the subject. In some embodiments, the classification is binary (e.g., positive or negative, somatic or germline, etc.) or has more levels of classification (e.g., a scale from 1 to 10 or 0 to 1). In some embodiments, the terms “cutoff” and “threshold” refer to predetermined numbers used in an operation. In one example, a cutoff size refers to a size above which fragments are excluded. In some embodiments, a threshold value is a value above or below which a particular classification applies. Either of these terms can be used in either of these contexts.

As used herein, the terms “control sample,” “reference sample,” and “normal sample” refer to a sample from a subject that does not have a particular condition or is otherwise healthy. In an example, a method as disclosed herein can be performed on a subject having a tumor, where the reference sample is a sample taken from a healthy tissue of the subject. A reference sample can be obtained from the subject, or from a database. The reference sample can be, e.g., a reference genome that is used to map sequence reads obtained from sequencing a sample from the subject. A reference genome can refer to a haploid or diploid genome to which sequence reads from the biological sample and a constitutional sample can be aligned and compared. An example of a constitutional sample can be DNA of white blood cells obtained from the subject. For a haploid genome, there can be one nucleotide at each locus. For a diploid genome, heterozygous loci can be identified; each heterozygous locus can have two alleles, where either allele can allow a match for alignment to the locus.

As used herein, the term “genomic position” or “locus” refers to a position (e.g., a site) within a genome, e.g., on a particular chromosome. In some embodiments, a genomic position (e.g., locus) refers to a single nucleotide position, on a particular chromosome, within a genome. In some embodiments, a genomic position refers to a group of nucleotide positions within a genome. In some embodiments, a genomic position refers to one or more genomic coordinates and/or a span of genomic coordinates (e.g., within a reference sequence or genome). For instance, in some embodiments, a genomic position is used to denote or identify a genomic region. In some instances, a genomic position is characterized by a mutation (e.g., substitution, insertion, deletion, inversion, or translocation) of consecutive nucleotides within a cancer genome. In some instances, a genomic position is a gene, a sub-genic structure (e.g., a regulatory element, exon, intron, or combination thereof), or a predefined span of a chromosome. Because normal mammalian cells have diploid genomes, a normal mammalian genome (e.g., a human genome) will generally have two copies of every genomic position (e.g., locus) in the genome, or at least two copies of every genomic position (e.g., locus) located on the autosomal chromosomes, e.g., one copy on the maternal autosomal chromosome and one copy on the paternal autosomal chromosome.

As disclosed herein, the terms “genomic region” or “chromosomal region” refer to any contiguous or non-contiguous portion of a genome. Genomic regions can also refer to, for example, as a bin, a partition, a genomic portion, a portion of a reference genome, a portion of a chromosome and the like. In some embodiments, a genomic region is based on a particular length of the genomic sequence. For example, in some embodiments, a method can include analysis of multiple mapped sequence reads to a plurality of genomic regions. Genomic regions can be approximately the same length or different lengths. In some embodiments, genomic regions of different lengths are adjusted or weighted. In some embodiments, a genomic region is about 3 base pairs (bp) to about 100 bp, about 0.1 kilobases (kb) to about 10 kb, about 10 kb to about 500 kb, about 20 kb to about 400 kb, about 30 kb to about 300 kb, about 40 kb to about 200 kb, and sometimes about 50 kb to about 100 kb. In some embodiments, a genomic region is about 100 kb to about 200 kb. A genomic region is not limited to contiguous runs of sequence. Thus, genomic regions can be made up of contiguous and/or non-contiguous sequences. A genomic region is not limited to a single chromosome. In some embodiments, a genomic region includes all or part of one chromosome or all or part of two or more chromosomes. In some embodiments, genomic regions may span one, two, or more entire chromosomes. In addition, the genomic regions may span joint or disjointed portions of multiple chromosomes.

As used herein, the term “measure of central tendency” refers to a central or representative value for a distribution of values. Non-limiting examples of measures of central tendency include an arithmetic mean, weighted mean, midrange, midhinge, trimean, geometric mean, geometric median, Winsorized mean, median, and mode of the distribution of values.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that's not cytosine; however, these are rarer occurrences. In this present disclosure, methylation is discussed in reference to CpG sites for the sake of clarity. Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

Various challenges arise in the identification of anomalously methylated cfDNA fragments. First, in some instances, determining a subject's cfDNA to be anomalously methylated holds weight in comparison with a group of control subjects, such that if the control group is small in number, the determination loses confidence with the small control group. Additionally, among a group of control subjects, methylation status can vary which can be difficult to account for when determining a subject's cfDNA to be anomalously methylated. On another note, in some instances, methylation of a cytosine at a CpG site causally influences methylation at a subsequent CpG site.

The principles described herein are equally applicable for the detection of methylation in a non-CpG context, including non-cytosine methylation. Further, the methylation state vectors may contain elements that are generally vectors of sites where methylation has or has not occurred (even if those sites are not CpG sites specifically). With that substitution, the remainder of the processes described herein are the same, and consequently, the inventive concepts described herein are applicable to those other forms of methylation.

In some embodiments, methylation levels of a nucleic acid fragment are provided using Beta-values and/or M-values, both of which provide a measure of differential methylation at a given CpG site or sites. For instance, the Beta-value is defined as the ratio of intensities between methylated alleles and the sum of all (methylated and unmethylated) alleles (e.g., for a given CpG site). Intensities can be determined by interrogating the respective CpG site(s) using methylated and unmethylated probes in a methylation assay (e.g., an Illumina methylation assay). The Beta-value statistic results in a number between 0 and 1, or 0 and 100%. Under ideal conditions, a value of zero indicates that all copies of the CpG site in the sample were completely unmethylated (no methylated molecules were measured) and a value of one indicates that every copy of the site was methylated. The M-value is defined as the log 2 ratio of the intensities between methylated alleles and unmethylated alleles (e.g., for a given CpG site). Intensities used for M-value estimation can be determined by interrogating the respective CpG site(s) using methylated and unmethylated probes in a methylation assay (e.g., an Illumina methylation assay). An M-value close to 0 indicates a similar intensity between the methylated and unmethylated probes, which generally means that the CpG site is about half-methylated. Positive M-values generally mean that a greater number of fragments are methylated than unmethylated, while negative M-values mean the opposite (a greater number of fragments are unmethylated than methylated). In some embodiments, the intensity data is normalized (e.g., by Illumina GenomeStudio or some other external normalization algorithm) prior to Beta-value or M-value estimation. Further details on Beta-values and M-values are provided in Du et al., “Comparison of Beta-value and M-value methods for quantifying methylation levels by microarray analysis,” BMC Bioinformatics 2010, 11:587, which is hereby incorporated by reference herein in its entirety.

As used herein, the term “methylation index” for each genomic site (e.g., a CpG site, a region of DNA where a cytosine nucleotide is followed by a guanine nucleotide in the linear sequence of bases along its 5′→3′ direction) refers to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density and proportion of methylated cytosines are examples of “methylation levels.”

As used herein, the term “methylation pattern” or “methylation state vector” refers to a sequence of methylation states for one or more CpG sites. Methylation states include, but are not limited to, methylated (e.g., represented as “M”) and unmethylated (e.g., represented as “U”). For example, a methylation pattern spanning 5 CpG sites may be represented as “MMMMM” or “UUUUU”, where each discrete symbol represents a methylation state at a single CpG site. A methylation pattern may or may not correspond to a specific genomic location and/or a specific one or more CpG sites in a reference genome.

As used interchangeably herein, the term “node,” “neuron,” “unit,” “hidden neuron,” “hidden unit,” or the like, refers to a unit of a neural network that accepts input and provides an output via an activation function and one or more parameters (e.g., weights and/or hyperparameters). For example, a node can accept one or more inputs from a prior layer and provide an output that serves as an input for a subsequent layer. In some embodiments, a neural network comprises one output node. In some embodiments, a neural network comprises a plurality of output nodes. Generally, the output is a prediction value, such as a probability or likelihood, a binary determination (e.g., a presence or absence, a positive or negative result, an identification of somatic or germline variant, etc.), and/or a label (e.g., a classification) of a condition of interest such as a cancer condition. For single-class classification models, the output can be a likelihood of an input dataset (e.g., of a biological sample and/or subject) having a condition (e.g., a label or class). For multi-class classification models, multiple prediction values can be generated, with each prediction value indicating the likelihood of an input dataset for each condition of interest. In some embodiments, a node is associated with a parameter that contributes to the output of the neural network, determined based on the activation function. In some embodiments, the node is initialized with arbitrary parameters (e.g., randomized weights). In some alternative embodiments, the node is initialized with a predetermined set of parameters.

As used herein, the term “normalize” refers to the transformation of a value or a set of values to a common frame of reference for comparison purposes. For example, when a diagnostic ctDNA level is “normalized” with a baseline ctDNA level, the diagnostic ctDNA level is compared to the baseline ctDNA level so that the amount by which the diagnostic ctDNA level differs from the baseline ctDNA level can be determined.

As used interchangeably herein, the terms “nucleic acid” and “nucleic acid molecule” refer to nucleic acids of any composition form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments, nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of RNA or DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine, and deoxythymidine. For RNA, the base cytosine is replaced with uracil and the sugar 2′ position includes a hydroxyl moiety. A nucleic acid may be prepared using a nucleic acid obtained from a subject as a template.

As used herein, the term “nucleic acid fragment sequence” or “nucleic acid fragment” refers to all or a portion of a polynucleotide sequence of at least three consecutive nucleotides. In the context of sequencing nucleic acid molecules found in a biological sample, the term “nucleic acid fragment sequence” refers to the sequence of a nucleic acid fragment (e.g., a nucleic acid molecule fragment) that is found in the biological sample or a representation thereof (e.g., an electronic representation of the sequence). Sequencing data (e.g., raw or corrected sequence reads from whole-genome sequencing, targeted sequencing, whole-genome bisulfate sequencing, targeted methylation sequencing, etc.) from a unique nucleic acid fragment (e.g., a cell-free nucleic acid molecule) are used to determine the sequence of a nucleic acid fragment. Such sequence reads, which in fact may be obtained from sequencing of PCR duplicates of the original nucleic acid fragment, therefore “represent” or “support” the nucleic acid fragment sequence. There may be a plurality of sequence reads that each represents or supports a particular nucleic acid fragment in a biological sample (e.g., PCR duplicates), however, there may be one nucleic acid fragment sequence for the particular nucleic acid fragment. In some embodiments, duplicate sequence reads generated for the original nucleic acid fragment are combined or removed (e.g., collapsed into a single sequence, e.g., the nucleic acid fragment sequence). Accordingly, when determining metrics relating to a population of nucleic acid fragments, in a sample, that each encompass a particular locus (e.g., an abundance value for the locus or a metric based on a characteristic of the distribution of the fragment lengths), the nucleic acid fragment sequences for the population of nucleic acid fragments, rather than the supporting sequence reads (e.g., which may be generated from PCR duplicates of the nucleic acid fragments in the population), can be used to determine the metric. This is because, in such embodiments, one copy of the sequence is used to represent the original (e.g., unique) nucleic acid fragment (e.g., unique nucleic acid molecule fragment). It is noted that the nucleic acid fragment sequences for a population of nucleic acid fragments may include several identical sequences, each of which represents a different original nucleic acid fragment, rather than duplicates of the same original nucleic acid fragment. In some embodiments, a cell-free nucleic acid is referred to as a nucleic acid fragment.

As used herein, the term “positive predictive value,” “PPV,” or “precision” refers to the likelihood that an output (e.g., a variant classification) is correctly called by a prediction algorithm. PPV can be expressed as (number of true positives)/(number of false positives+number of true positives).

As used herein, the term “reference allele” refers to the sequence of one or more nucleotides at a genomic position that is either the predominant allele represented at that genomic position within the population of the species (e.g., the “wild-type” sequence), or an allele that is predefined within a reference genome for the species.

As disclosed herein, the term “reference genome” or “genome” refers to any known, sequenced, or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

The terms “sequence reads” or “reads,” used interchangeably herein, refer to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130 bp, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more. Nanopore sequencing, for example, can provide sequence reads that vary in size from tens to hundreds to thousands of base pairs. Illumina parallel sequencing can provide sequence reads vary to a lesser extent (e.g., where most sequence reads are of a length of about 200 bp or less). A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes (e.g., in hybridization arrays or capture probes) or amplification techniques, such as the polymerase chain reaction (PCR) or linear amplification using a single primer or isothermal amplification.

As disclosed herein, the terms “sequencing,” “sequence determination,” and the like refer generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sensitivity,” “recall,” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As disclosed herein, the term “subject,” “reference subject,” “training subject,” or “test subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. The terms “subject” and “patient” are used interchangeably herein and refer to a human or non-human animal who is known to have, or potentially has, a medical condition or disorder, such as, e.g., a cancer. In some embodiments, a subject is a male or female of any stage (e.g., a man, a woman, or a child).

A subject from whom a sample is taken or who is treated by any of the methods or compositions described herein can be of any age and can be an adult, infant or child. In some cases, the subject, e.g., patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old). A particular class of subjects, e.g., patients that can benefit from a method of the present disclosure is subjects, e.g., patients over the age of 40.

Another particular class of subjects, e.g., patients that can benefit from a method of the present disclosure is pediatric patients, who can be at higher risk of chronic heart symptoms. Furthermore, a subject, e.g., a patient from whom a sample is taken, or is treated by any of the methods or compositions described herein, can be male or female.

As used herein, the term “tissue” corresponds to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “tumor mutational burden” (TMB) refers to a measure of the mutations in a cancer per unit of the patient's genome (e.g., a measurement of mutations carried by tumor cells). For example, a tumor mutational burden can be expressed as a measure of central tendency (e.g., an average) of the number of somatic variants per million base pairs in the genome. In some embodiments, a tumor mutational burden refers to a measure of one or more types of possible mutations, e.g., one or more of SNVs, MNVs, indels, or genomic rearrangements. In some embodiments, a tumor mutational burden refers to a subset of one or more types of possible mutations, such as a non-synonymous mutation (e.g., a mutation that alters the amino acid sequence of an encoded protein). In other embodiments, for example, a tumor mutational burden refers to the number of one or more types of mutations that occur in protein coding sequences (e.g., regardless of whether they change the amino acid sequence of the encoded protein). As an example, in some embodiments, a tumor mutational burden is calculated by dividing the number of mutations (e.g., all variants and/or non-synonymous variants) identified in the sequencing data by the size (e.g., in megabases, of an electronic file) of a capture probe panel used for targeted sequencing. Other methods for calculating tumor mutation burden in liquid biopsy samples and/or solid tissue samples are known in the art.

As used herein, the term “tumor fraction” refers to the fraction of nucleic acid molecules in a sample that originates from a cancerous tissue of the subject, rather than from a noncancerous tissue (e.g., a germline or hematopoietic tissue). Tumor fraction can be measured using solid tissue samples or liquid biopsy samples. For instance, as used herein, the term “circulating tumor fraction” refers to the fraction of cell-free nucleic acid molecules in a liquid biopsy sample that originates from a cancerous tissue of the subject, rather than from a noncancerous tissue. However, estimating tumor fraction from liquid biopsy samples can be challenging because such samples generally have lower tumor fractions relative to solid tumor samples and because targeted panels used for liquid biopsy sequencing are typically small.

Software packages for calculating tumor fraction include, for example, PureCN, which is designed to estimate tumor purity from targeted short-read sequencing data of solid tumor samples, and FACETS, which is designed to estimate tumor fraction from sequencing data of solid tumor samples. In addition, the ichorCNA package applies a probabilistic model to normalized read coverages from ultra-low pass whole genome sequencing data of cell-free DNA to estimate tumor fraction in the liquid biopsy sample. Tumor fraction can also be determined using a Maximum Likelihood model based on the copy number of an allele in the sample and variant allele frequency in paired-control samples.

Methods for determining tumor fraction and tumor mutational burden are described in further detail in U.S. patent application Ser. No. 17/185,885, filed Feb. 25, 2021, entitled “Systems and Methods for Calling Variants using Methylation Sequencing Data,” and PCT Application No. PCT/US2021/019746, filed February 2021, entitled “Systems and Methods for Calling Variants using Methylation Sequencing Data,” each of which is hereby incorporated herein by reference in its entirety.

As used herein the term “untrained classifier” refers to a classifier that has not been trained on a target dataset. For instance, consider the case of a first canonical set of methylation state vectors and a second canonical set of methylation state vectors discussed below. The respective canonical sets of methylation state vectors are applied as collective input to an untrained classifier, in conjunction with the cell source of each respective reference subject represented by the first canonical set of methylation state vectors (hereinafter “primary training dataset”) to train the untrained classifier on cell source thereby obtaining a trained classifier. Moreover, it will be appreciated that the term “untrained classifier” does not exclude the possibility that transfer learning techniques are used in such training of the untrained classifier. In instances where transfer learning is used, the untrained classifier described above is provided with additional data over and beyond that of the primary training dataset. That is, in non-limiting examples of transfer learning embodiments, the untrained classifier receives (i) canonical sets of methylation state vectors and the cell source labels of each of the reference subjects represented by canonical sets of methylation state vectors (“primary training dataset”) and (ii) additional data. Typically, this additional data is in the form of coefficients (e.g., regression coefficients) that were learned from another, auxiliary training dataset. Moreover, while a description of a single auxiliary training dataset has been disclosed, it will be appreciated that there is no limit on the number of auxiliary training datasets that may be used to complement the primary training dataset in training the untrained classifier in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The coefficients learned from the first auxiliary training dataset (by application of a classifier such as regression to the first auxiliary training dataset) may be applied to the second auxiliary training dataset using transfer learning techniques (e.g., the above described two-dimensional matrix multiplication), which in turn may result in a trained intermediate classifier whose coefficients are then applied to the primary training dataset and this, in conjunction with the primary training dataset itself, is applied to the untrained classifier. Alternatively, a first set of coefficients learned from the first auxiliary training dataset (by application of a classifier such as regression to the first auxiliary training dataset) and a second set of coefficients learned from the second auxiliary training dataset (by application of a classifier such as regression to the second auxiliary training dataset) may each individually be applied to a separate instance of the primary training dataset (e.g., by separate independent matrix multiplications) and both such applications of the coefficients to separate instances of the primary training dataset in conjunction with the primary training dataset itself (or some reduced form of the primary training dataset such as principal components or regression coefficients learned from the primary training set) may then be applied to the untrained classifier in order to train the untrained classifier. In either example, knowledge regarding cell source (e.g., cancer type, etc.) derived from the first and second auxiliary training datasets is used, in conjunction with the cell source labeled primary training dataset), to train the untrained classifier.

As used herein, the terms “variant” or “mutation” refer to a detectable change in the genetic material of one or more cells. A variant or mutation can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene or a large span of a chromosome) (CNV), a partial or complete change in the ploidy of the cell, and/or changes in the epigenetic information of a genome, such as altered DNA methylation patterns. For example, a single nucleotide variant or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.” In some embodiments, a variant is a change in the genetic information of the cell relative to a particular reference genome or one or more “normal” or “reference” alleles found in the population of the species of the subject. In some embodiments, a variant is a change in the genetic information of the cell relative to a reference cell or tissue, such as a “normal” or “healthy” tissue in the subject. In some embodiments, a variant is a germline mutation or a somatic mutation.

In some instances, a variant refers to a cancer metric derived from nucleic acid sequencing data. In some instances, a variant refers to tumor mutational burden, microsatellite instability (MSI) status, ploidy, or tumor fraction. In some instances, a variant refers to a fusion, an amplification, and/or an isoform.

As used herein, the term “variant allele” refers to a sequence of one or more nucleotides at a genomic position that is either not the predominant allele represented at that genomic position within the population of the species (e.g., not the “wild-type” sequence), or not an allele that is predefined within a reference genome for the species.

As used herein, the term “parameter” refers to any coefficient or, similarly, any value of an internal or external element (e.g., a weight and/or hyperparameter) in a model, classifier, or algorithm that can affect (e.g., modify, tailor, and/or adjust) one or more inputs, outputs, and/or functions in the model, classifier, or algorithm. For example, in some embodiments, a parameter refers to any coefficient, weight, and/or hyperparameter that can be used to control, modify, tailor, and/or adjust the behavior, learning and/or performance of a model. In some embodiments, a parameter has a fixed value. In some embodiments, a value of a parameter is manually and/or automatically adjustable. In some embodiments, a value of a parameter is modified by a classifier validation and/or training process (e.g., by error minimization and/or backpropagation methods, as described elsewhere herein).

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are used to implement a methodology in accordance with the features described herein.

Exemplary System Embodiments

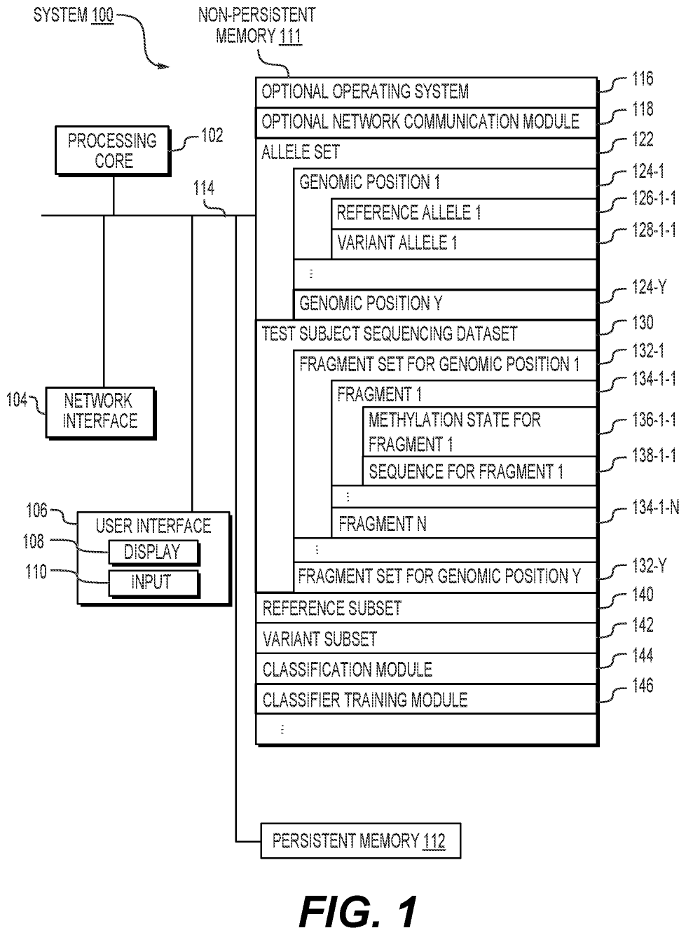

Details of an exemplary system are now described in conjunction with FIG. 1. FIG. 1 is a block diagram illustrating a system 100 in accordance with some implementations. System 100 in some implementations includes one or more processing units CPU(s) 102 (also referred to as processors or processing core), one or more network interfaces 104, user interface 106, non-persistent memory 111, persistent memory 112, and one or more communication buses 114 for interconnecting these components. One or more communication buses 114 optionally include circuitry (sometimes called a chipset) that interconnects and controls communications between system components. Non-persistent memory 111 typically includes high-speed random access memory, such as DRAM, SRAM, DDR RAM, ROM, EEPROM, flash memory, whereas persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid-state storage devices. Persistent memory 112 optionally includes one or more storage devices remotely located from the CPU(s) 102. Persistent memory 112, and the non-volatile memory device(s) within non-persistent memory 112, comprise non-transitory computer-readable storage medium. In some implementations, non-persistent memory 111 or alternatively non-transitory computer-readable storage medium stores the following programs, modules and data structures, or a subset thereof, sometimes in conjunction with persistent memory 112:

optional instructions, programs, data, or information associated with optional operating system 116, which includes procedures for handling various basic system services and for performing hardware dependent tasks;

instructions, programs, data, or information associated with an optional network communication module (or instructions) 118 for connecting the system 100 with other devices, or a communication network;

instructions, programs, data, or information associated with an allele set 122 that stores, for a genomic position 124 (optionally, a respective genomic position in a plurality of genomic positions 124-1 . . . 124-Y), an identification of a reference allele 126 (e.g., 126-1-1) and an identification of a variant allele 128 (e.g., 128-1-1);

a sequencing dataset 130 derived from a biological sample (e.g., a liquid biological sample) obtained from a test subject that includes a respective set of nucleic acid fragments that map onto the genomic position 132 (optionally, a respective fragment set for each genomic position in a plurality of genomic positions 132-1 . . . 132-Y) and, for each nucleic acid fragment 134 (e.g., 134-1-1 . . . 134-1-N) in the set of nucleic acid fragments, a respective methylation state 136 (e.g., 136-1-1) and a respective sequence for the nucleic acid fragment 138 (e.g., 138-1-1);

a reference subset 140 that includes each nucleic acid fragment 134 in the respective set of nucleic acid fragments 132 that has the reference allele at the genomic position 124, where a respective nucleic acid fragment is assigned to the reference subset using the identification of the reference allele 126 at the genomic position and the respective sequence 138 of the nucleic acid fragment;

a variant subset 142 that includes each nucleic acid fragment 134 in the respective set of nucleic acid fragments 132 that has the variant allele at the genomic position 124, where a respective nucleic acid fragment is assigned to the variant subset using the identification of the variant allele 128 at the genomic position and the respective sequence 138 of the nucleic acid fragment;

a classification module 144 for applying, to a trained binary classifier, at least (i) one or more indications of methylation state across the methylation state 136 of each nucleic acid fragment sequence in the variant subset and (ii) an indication of a number of nucleic acid fragment sequences in the reference subset 140 versus a number of nucleic acid fragment sequences in the variant subset 142, thereby obtaining from the trained binary classifier an identification of the variant allele at the genomic position in the test subject as somatic or germline; and

optionally, a classifier training module 146 for training the binary classifier used for the identification of the variant allele at the genomic position.

In some implementations, one or more of the above-identified elements are stored in one or more of the previously mentioned memory devices and correspond to a set of instructions for performing a function described above. The above-identified modules, data, or programs (e.g., sets of instructions) may not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of visualization system 100, that is addressable by visualization system 100 so that visualization system 100 may retrieve all or a portion of such data.

Although FIG. 1 depicts a “system 100,” the figure is intended more as functional description of the various features which may be present in computer systems than as a structural schematic of the implementations described herein. In practice, items shown separately could be combined and some items can be separated. Moreover, although FIG. 1 depicts certain data and modules in non-persistent memory 111, some or all of these data and modules may be in persistent memory 112.

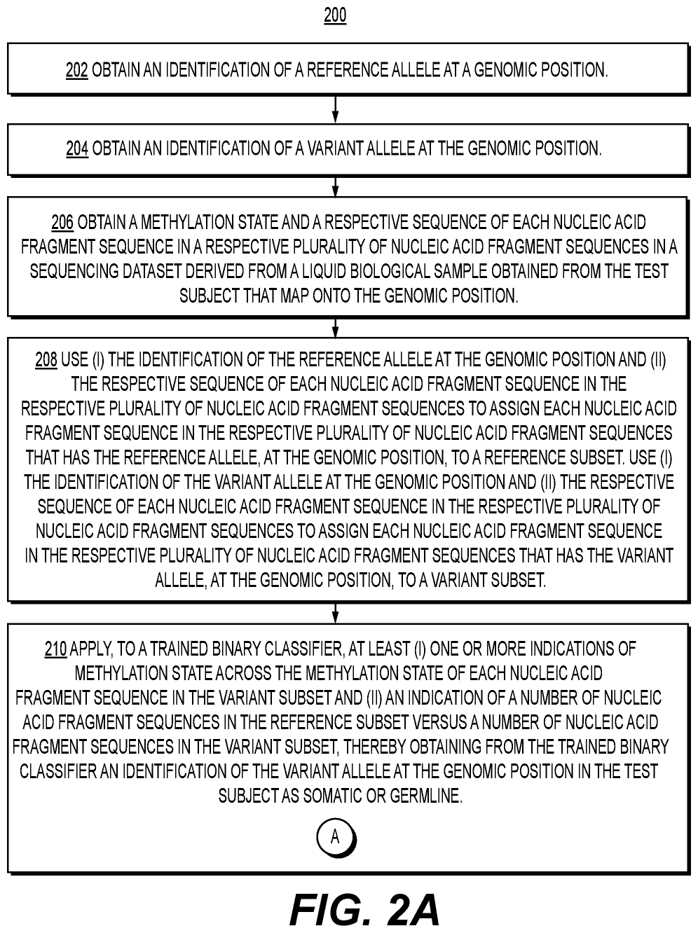

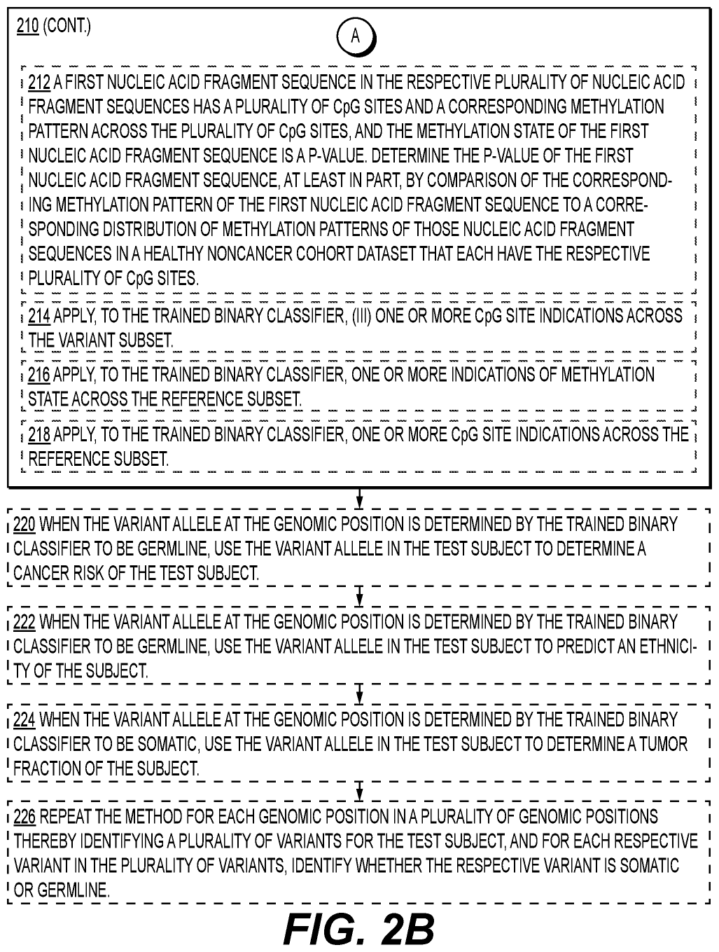

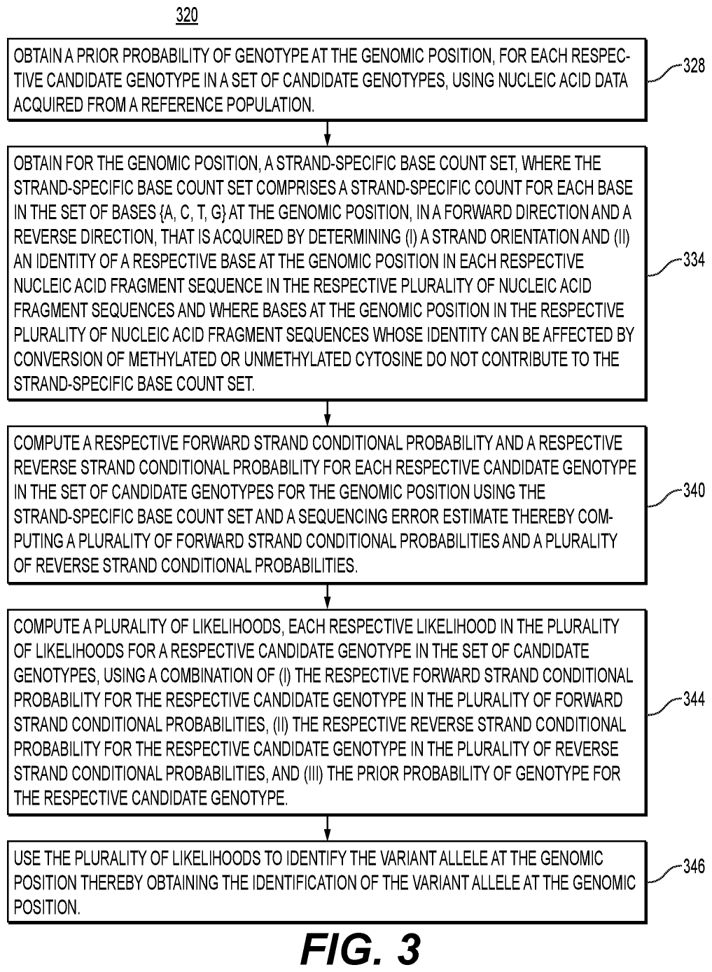

While a system in accordance with the present disclosure has been disclosed with reference to FIG. 1, methods in accordance with the present disclosure are now detailed with reference to FIGS. 2A, 2B and 3. Any of the disclosed methods can make use of any of the assays or algorithms disclosed in U.S. patent application Ser. No. 15/793,830, filed Oct. 25, 2017, and/or International Patent Publication No. WO 2018/081130, entitled “Methods and Systems for Tumor Detection,” each of which is hereby incorporated by reference, in order to determine a cancer condition in a test subject or a likelihood that the subject has the cancer condition. For instance, any of the disclosed methods can work in conjunction with any of the disclosed methods or algorithms disclosed in U.S. patent application Ser. No. 15/793,830, filed Oct. 25, 2017, and/or International Patent Publication No. WO 2018/081130, entitled “Methods and Systems for Tumor Detection.”

Identifying Variant Alleles

Referring to FIGS. 2A and 2B, provided herein is a method 200 of identifying a variant allele at a genomic position in a test subject as somatic or germline.

Subjects and samples.

In some embodiments, the test subject is mammalian. In some embodiments, the test subject is human. In some embodiments, the test subject is a patient with a cancer.

In some embodiments, the method comprises obtaining a biological sample from the test subject. In some embodiments, the biological sample is one of a plurality of biological samples obtained from the test subject (e.g., a plurality of replicates and/or a plurality of samples including a matched tumor sample and a matched normal sample). In some embodiments, a plurality of biological samples is obtained from the test subject concurrently or at intervals over a period of time (e.g., for serial analysis). For example, in some such embodiments, the time between obtaining biological samples from the test subject is at least 1 day, at least 2 days, at least 1 week, at least 2 weeks, at least 1 month, at least 2 months, at least 3 months, at least 4 months, at least 6 months, or at least 1 year.

In some embodiments, the biological sample is obtained from any tissue, organ or fluid from the subject.

In some embodiments, the biological sample is a liquid biological sample (e.g., a liquid biopsy sample). In some embodiments, the liquid biological sample comprises blood, whole blood, plasma, serum, urine, cerebrospinal fluid, fecal, saliva, sweat, tears, pleural fluid, pericardial fluid, or peritoneal fluid of the test subject. In some embodiments, the liquid biological sample consists of blood, whole blood, plasma, serum, urine, cerebrospinal fluid, fecal, saliva, sweat, tears, pleural fluid, pericardial fluid, or peritoneal fluid of the test subject.

In some embodiments, the biological sample is a tissue sample. In some embodiments, the tissue sample is a tumor sample from the test subject. In some embodiments, the tumor sample is of a homogenous tumor. In some embodiments, the tumor sample is of a heterogenous tumor.

In some embodiments, the biological sample comprises a respective plurality of nucleic acid fragments. In some embodiments, the respective plurality of nucleic acid fragments comprises cell-free nucleic acid fragments (e.g., cfDNA). In some embodiments, the respective plurality of nucleic acid fragments comprise cell-free nucleic acid fragments (e.g., cfDNA). In some embodiments, a nucleic acid fragment in the plurality of nucleic acid fragments includes any of the embodiments for nucleic acids disclosed herein (see, for example, Definitions: Nucleic acids).

In some embodiments, the biological sample comprises a mixture of nucleic acid molecules derived from diseased cells and nucleic acid molecules derived from healthy cells. For instance, in some embodiments, the biological sample is a blood sample comprising cfDNA derived from tumor cells (e.g., ctDNA), cfDNA derived from normal cells, and/or normal cells (e.g., white blood cells).

In some embodiments, the biological sample is processed to extract the nucleic acids in preparation for sequencing analysis. By way of a non-limiting example, in some embodiments, cell-free nucleic acid fragments are extracted from a liquid biological sample (e.g., a blood sample) collected from a subject in K2 EDTA tubes. In the case where the biological samples are blood, by way of nonlimiting example, the samples are processed within two hours of collection by double spinning of the biological sample first at ten minutes at 1000 g, and then the resulting plasma is spun ten minutes at 2000 g. The plasma is then stored in 1 ml aliquots at −80° C. In this way, a suitable amount of plasma (e.g., 1-5 ml) is prepared from the biological sample for the purposes of cell-free nucleic acid extraction. In some embodiments, cell-free nucleic acid is extracted using the QIAamp Circulating Nucleic Acid kit (Qiagen) and eluted into DNA Suspension Buffer (Sigma). In some embodiments, the purified cell-free nucleic acid is stored at −20° C. until use.

Other equivalent methods can be used to prepare and/or extract nucleic acid fragments (e.g., cell-free nucleic acid fragments) from biological samples for the purpose of sequencing, and all such methods are within the scope of the present disclosure.

In some embodiments, the respective plurality of nucleic acid fragments (e.g., cell-free nucleic acid fragments) from the test subject comprises 100 or more nucleic acid fragments, 1000 or more nucleic acid fragments, 10,000 or more nucleic acid fragments, 20,000 or more nucleic acid fragments, 50,000 or more nucleic acid fragments, 100,000 or more nucleic acid fragments, 200,000 or more nucleic acid fragments, 500,000 or more nucleic acid fragments, 1,000,000 or more nucleic acid fragments, 2,000,000 or more nucleic acid fragments, 5,000,000 or more nucleic acid fragments, 10,000,000 or more nucleic acid fragments, or 50,000,000 or more nucleic acid fragments. In some embodiments, the nucleic acid fragments (e.g., cell-free nucleic acid fragments) from the test subject comprises no more than 50,000,000, no more than 10,000,000, no more than 5,000,000, no more than 2,000,000, no more than 1,000,000, no more than 500,000, no more than 200,000, no more than 100,000, no more than 50,000, no more than 20,000, no more than 10,000, or no more than 1000 nucleic acid fragments. In some embodiments, the nucleic acid fragments (e.g., cell-free nucleic acid fragments) from the test subject comprises from 100 to 1000, from 1000 to 10,000, from 10,000 to 100,000, from 100,000 to 1,000,000, from 1,000,000 to 10,000,000, or from 10,000,000 to 50,000,000 nucleic acid fragments. In some embodiments, the nucleic acid fragments (e.g., cell-free nucleic acid fragments) from the test subject falls within another range starting no lower than 100 nucleic acid fragments and ending no higher than 50,000,000 nucleic acid fragments.

In some embodiments, the nucleic acid fragments obtained from a biological sample are cell-free nucleic acids derived from tumor cells (e.g., ctDNA). In some embodiments, the nucleic acid fragments obtained from a biological sample are cell-free nucleic acids derived from normal cells. In some embodiments, the nucleic acid fragments obtained from a biological sample are obtained directly from tumor cells (e.g., solid tumor biopsy). In some embodiments, the nucleic acid fragments obtained from a biological sample are obtained directly from normal cells (e.g., healthy tissue and/or white blood cells).

In some embodiments, the nucleic acid fragments that are obtained from a biological sample are any form of nucleic acid defined in the present disclosure (e.g., cell-free nucleic acid fragments), or a combination thereof (see, for example, Definitions: Nucleic acids). For example, in some embodiments, the nucleic acid that is obtained from a biological sample is a mixture of RNA and DNA (e.g., cell-free RNA and/or cell-free DNA).

In some embodiments, the method comprises sequencing the respective plurality of nucleic acid molecules in the biological sample obtained from the test subject, thus obtaining a respective plurality of nucleic acid fragment sequences. For instance, in some embodiments, the biological sample is a liquid biological sample and each respective nucleic acid fragment sequence in the respective plurality of nucleic acid fragment sequences represents all or a portion of a respective cell-free nucleic acid molecule in a population of cell-free nucleic acid molecules in the liquid biological sample. In some embodiments, alternately or additionally, the biological sample is a tissue sample and each respective nucleic acid fragment sequence in the respective plurality of nucleic acid fragment sequences represents all or a portion of a respective nucleic acid molecule in a population of nucleic acid molecules in the tissue sample. Non-limiting embodiments of methods for obtaining nucleic acid fragment sequences are detailed below in the following sections (see, “Obtaining nucleic acid fragment sequences”).

Reference and Variant Alleles.

Referring to Blocks 202 and 204, the method further includes obtaining an identification of a reference allele at the genomic position and obtaining an identification of the variant allele at the genomic position.

In some embodiments, the variant allele is an insertion, a deletion, a single nucleotide variant (SNV) or a single nucleotide polymorphism (SNP). In some embodiments, the variant allele is any variant or mutation defined herein (see, Definitions: Variant).

In some embodiments, the genomic position is any genomic position or locus defined herein (see, Definitions: Genomic position). For example, in some embodiments, the genomic position is a single base position and the variant is a single nucleotide variant (SNV) or single nucleotide polymorphism (SNP). In some embodiments, the genomic position is two or more base positions, and the variant is an insertion or a deletion. In some embodiments, the genomic position is a portion or region of a reference genome.

In some embodiments, the genomic position is associated with a clinically actionable variant. For instance, in some embodiments, the genomic position indicates a genomic variant that is associated with an increased risk for a cancer condition, such as an increased severity, likelihood of progression, and/or an indication of a type of cancer (e.g., a KRAS mutation in lung cancer). In some such embodiments, the presence and/or identification of a respective genomic variant can influence clinical decision-making, such as treatment recommendation, clinical trial enrollment, and other physician actions. In some embodiments, a clinically actionable variant is a somatic variant or a germline variant. In some embodiments, a clinically actionable variant is associated with a gene.

In some embodiments, the genomic position comprises all or part of a gene or is characterized by a mutation in a gene. In some embodiments, the gene is a cancer gene, e.g., where a dysfunction in the gene is associated with a cancer. Non-limiting examples of dysfunction include genomic alterations (e.g., mutations and/or variant alleles), dysregulation, changes in activity, changes in expression, and/or changes in epigenetic modifications such as methylation. In some embodiments, cancer genes include known cancer genes, candidate cancer genes, oncogenes, tumor suppressor genes, and/or tissue-specific genes (e.g., genes associated with specific cancer types). In some embodiments, cancer genes are obtained based on annotations from sequencing screens, manual curation by experts, and/or experimental data. In some embodiments, cancer genes are obtained from a database, such as the Network of Cancer Genes (NCG), the International Cancer Genome Consortium (ICGC), the Cancer Genome Atlas (TCGA), COSMIC, DoCM, DriverDB, the Cancer Genome Interpreter, OncoKB, cBIOPortal, the Cancer Gene Census (CGC), ONGene, TSGene, and/or CoReCG.

In some embodiments, a cancer gene is selected from the group consisting of: A1CF, ABI1, ABL1, ABL2, ACKR3, ACSL3, ACSL6, ACVR1, ACVR1B, ACVR2A, AFDN, AFF1, AFF3, AFF4, AKAP9, AKT1, AKT2, AKT3, ALDH2, ALK, AMER1, ANK1, APC, APOBEC3B, AR, ARAF, ARHGAP26, ARHGAP5, ARHGEF10, ARHGEF10L, ARHGEF12, ARID1A, ARID1B, ARID2, ARNT, ASPSCR1, ASXL1, ASXL2, ATF1, ATIC, ATM, ATP1A1, ATP2B3, ATR, ATRX, AXIN1, AXIN2, B2M, BAP1, BARD1, BAX, BAZ1A, BCL10, BCL11A, BCL11B, BCL2, BCL2L12, BCL3, BCL6, BCL7A, BCL9, BCL9L, BCLAF1, BCOR, BCORL1, BCR, BIRC3, BIRC6, BLM, BMP5, BMPR1A, BRAF, BRCA1, BRCA2, BRD3, BRD4, BRIP1, BTG1, BTK, BUB1B, C15orf65, CACNA1D, CALR, CAMTA1, CANT1, CARD11, CARS, CASP3, CASP8, CASP9, CBFA2T3, CBFB, CBL, CBLB, CBLC, CCDC6, CCNB1IP1, CCNC, CCND1, CCND2, CCND3, CCNE1, CCR4, CCR7, CD209, CD274, CD28, CD74, CD79A, CD79B, CDC73, CDH1, CDH10, CDH11, CDH17, CDK12, CDK4, CDK6, CDKN1A, CDKN1B, CDKN2A, CDKN2C, CDX2, CEBPA, CEP89, CHCHD7, CHD2, CHD4, CHEK2, CHIC2, CHST11, CIC, CIITA, CLIP1, CLP1, CLTC, CLTCL1, CNBD1, CNBP, CNOT3, CNTNAP2, CNTRL, COL1A1, COL2A1, COL3A1, COX6C, CPEB3, CREB1, CREB3L1, CREB3L2, CREBBP, CRLF2, CRNKL1, CRTC1, CRTC3, CSF1R, CSF3R, CSMD3, CTCF, CTNNA2, CTNNB1, CTNND1, CTNND2, CUL3, CUX1, CXCR4, CYLD, CYP2C8, CYSLTR2, DAXX, DCAF12L2, DCC, DCTN1, DDB2, DDIT3, DDR2, DDX10, DDX3X, DDX5, DDX6, DEK, DGCR8, DICER1, DNAJB1, DNM2, DNMT1, DNMT3A, DROSHA, EBF1, ECT2L, EED, EGFR, EIF1AX, EIF3E, EIF4A2, ELF3, ELF4, ELK4, ELL, ELN, EML4, EP300, EPAS1, EPHA3, EPHA7, EPS15, ERBB2, ERBB3, ERBB4, ERC1, ERCC2, ERCC3, ERCC4, ERG, ESR1, ETNK1, ETV1, ETV4, ETV5, ETV6, EWSR1, EXT1, EXT2, EZH2, EZR, FAM131B, FAM135B, FAM46C, FAM47C, FANCA, FANCC, FANCD2, FANCE, FANCF, FANCG, FAS, FAT1, FAT3, FAT4, FBLN2, FBXO11, FBXW7, FCGR2B, FCRL4, FEN1, FES, FEV, FGFR1, FGFR10P, FGFR2, FGFR3, FGFR4, FH, FHIT, FIP1L1, FKBP9, FLCN, FLI1, FLNA, FLT3, FLT4, FNBP1, FOXA1, FOXL2, FOXO1, FOXO3, FOXO4, FOXP1, FOXR1, FSTL3, FUBP1, FUS, GAS7, GATA1, GATA2, GATA3, GLI1, GMPS, GNA11, GNAQ, GNAS, GOLGA5, GOPC, GPC3, GPC5, GPHN, GRIN2A, GRM3, H3F3A, H3F3B, HERPUD1, HEY1, HIF1A, HIP1, HIST1H3B, HIST1H4I, HLA-A, HLF, HMGA1, HMGA2, HNF1A, HNRNPA2B1, HOOK3, HOXA11, HOXA13, HOXA9, HOXC11, HOXC13, HOXD11, HOXD13, HRAS, HSP90AA1, HSP90AB1, ID3, IDH1, IDH2, IGF2BP2, IKBKB, IKZF1, IL2, IL21R, IL6ST, IL7R, IRF4, IRS4, ISX, ITGAV, ITK, JAK1, JAK2, JAK3, JAZF1, JUN, KAT6A, KAT6B, KAT7, KCNJ5, KDM5A, KDM5C, KDM6A, KDR, KDSR, KEAP1, KIAA1549, KIF5B, KIT, KLF4, KLF6, KLK2, KMT2A, KMT2C, KMT2D, KNL1, KNSTRN, KRAS, KTN1, LARP4B, LASP1, LCK, LCP1, LEF1, LEPROTL1, LHFPL6, LIFR, LMNA, LMO, LMO2, LPP, LRIG3, LRP1B, LSM14A, LYL1, LZTR1, MAF, MAFB, MALT1, MAML2, MAP2K1, MAP2K2, MAP2K4, MAP3K1, MAP3K13, MAPK1, MAX, MB21D2, MDM2, MDM4, MDS2, MECOM, MED12, MEN1, MET, MGMT, MITF, MKL1, MLF1, MLH1, MLLT1, MLLT10, MLLT11, MLLT3, MLLT6, MN1, MNX1, MPL, MSH2, MSH6, MSI2, MSN, MTCP1, MTOR, MUC1, MUC16, MUC4, MUTYH, MYB, MYC, MYCL, MYCN, MYD88, MYH11, MYH9, MYO5A, MYOD1, N4BP2, NAB2, NACA, NBEA, NBN, NCKIPSD, NCOA1, NCOA2, NCOA4, NCOR1, NCOR2, NDRG1, NF1, NF2, NFATC2, NFE2L2, NFIB, NFKB2, NFKBIE, NIN, NKX2-1, NONO, NOTCH1, NOTCH2, NPM1, NR4A3, NRAS, NRG1, NSD1, NSD2, NSD3, NT5C2, NTHL1, NTRK1, NTRK3, NUMA1, NUP214, NUP98, NUTM1, NUTM2A, NUTM2B, OLIG2, OMD, P2RY8, PABPC1, PAFAH1B2, PALB2, PATZ1, PAX3, PAX5, PAX7, PAX8, PBRM1, PBX1, PCBP1, PCM1, PDCD1LG2, PDGFB, PDGFRA, PDGFRB, PER1, PHF6, PHOX2B, PICALM, PIK3CA, PIK3CB, PIK3R1, PIM1, PLAG1, PLCG1, PML, PMS1, PMS2, POLD1, POLE, POLG, POLQ, POT1, POU2AF1, POU5F1, PPARG, PPFIBP1, PPM1D, PPP2R1A, PPP6C, PRCC, PRDM1, PRDM16, PRDM2, PREX2, PRF1, PRKACA, PRKAR1A, PRKCB, PRPF40B, PRRX1, PSIP1, PTCH1, PTEN, PTK6, PTPN11, PTPN13, PTPN6, PTPRB, PTPRC, PTPRD, PTPRK, PTPRT, PWWP2A, QKI, RABEP1, RAC1, RAD17, RAD21, RAD51B, RAF1, RALGDS, RANBP2, RAP1GDS1, RARA, RB1, RBM10, RBM15, RECQL4, REL, RET, RFWD3, RGPD3, RGS7, RHOA, RHOH, RMI2, RNF213, RNF43, ROBO2, ROS1, RPL10, RPL22, RPLS, RPN1, RSPO2, RSPO3, RUNX1, RUNX1T1, S100A7, SALL4, SBDS, SDC4, SDHA, SDHAF2, SDHB, SDHC, SDHD, SEPTS, SEPT6, SEPT9, SET, SETBP1, SETD1B, SETD2, SF3B1, SFPQ, SFRP4, SGK1, SH2B3, SH3GL1, SHTN1, SIRPA, SIX1, SIX2, SKI, SLC34A2, SLC45A3, SMAD2, SMAD3, SMAD4, SMARCA4, SMARCB1, SMARCD1, SMARCE1, SMC1A, SMO, SND1, SNX29, SOCS1, SOX2, SOX21, SOX9, SPECC1, SPEN, SPOP, SRC, SRGAP3, SRSF2, SRSF3, SS18, SS18L1, SSX1, SSX2, SSX4, STAG1, STAG2, STAT3, STATSB, STAT6, STIL, STK11, STRN, SUFU, SUZ12, SYK, TAF15, TAL1, TAL2, TBL1XR1, TBX3, TCEA1, TCF12, TCF3, TCF7L2, TCL1A, TEC, TERT, TET1, TET2, TFE3, TFEB, TFG, TFPT, TFRC, TGFBR2, THRAP3, TLX1, TLX3, TMEM127, TMPRSS2, TNC, TNFAIP3, TNFRSF14, TNFRSF17, TOP1, TP53, TP63, TPM3, TPM4, TPR, TRAF7, TRIM24, TRIM27, TRIM33, TRIP11, TRRAP, TSC1, TSC2, TSHR, U2AF1, UBRS, USP44, USP6, USPS, VAV1, VHL, VTI1A, WAS, WDCP, WIF1, WNK2, WRN, WT1, WWTR1, XPA, XPC, XPO1, YWHAE, ZBTB16, ZCCHC8, ZEB1, ZFHX3, ZMYM2, ZMYM3, ZNF331, ZNF384, ZNF429, ZNF479, ZNF521, ZNRF3, and ZRSR2.

Cancer genes are further detailed in Repana et al., 2019, “The Network of Cancer Genes (NCG): a comprehensive catalogue of known and candidate cancer genes from cancer sequencing screens,” Genome Biology 20:1, doi: 10.1186/s13059-018-1612-0, which is hereby incorporated herein by reference in its entirety.

In some embodiments, the genomic position is selected from a plurality of genomic positions. For example, in some embodiments, the systems and methods disclosed herein can used to identify a plurality of variant alleles at a corresponding plurality of genomic positions as somatic or germline. In some embodiments, the plurality of genomic positions comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 10,000, or at least 20,000 genomic positions. In some embodiments, the plurality of genomic positions comprises no more than 20,000, no more than 10,000, no more than 5000, no more than 4000, no more than 3000, no more than 2000, no more than 1000, no more than 900, no more than 800, no more than 700, no more than 600, no more than 500, no more than 400, no more than 300, no more than 200, no more than 100, no more than 90, no more than 80, no more than 70, no more than 60, no more than 50, or no more than 20 genomic positions. In some embodiments, the plurality of genomic positions is from 10 to 50, from 50 to 100, from 100 to 500, from 500 to 1000, from 1000 to 5000, from 5000 to 10,000, or from 10,000 to 20,000 genomic positions. In some embodiments, the plurality of genomic positions falls within another range starting no lower than 10 genomic positions and ending no higher than 20,000 genomic positions.

In some embodiments, a respective genomic position in the plurality of genomic positions is associated with a respective clinically actionable variant (e.g., a cancer gene). In some embodiments, each respective genomic position in the plurality of genomic positions is associated with a respective clinically actionable variant (e.g., a cancer gene). In some embodiments, the plurality of genomic positions is a panel of clinically actionable variants (e.g., cancer genes of interest).

Variant Calling.

Referring again to Blocks 202 and 204, in some embodiments, the identification of the reference allele at the genomic position is obtained from a reference genome. Reference genomes can include any of the embodiments disclosed herein (see, Definitions: Reference genome).

In some embodiments, the obtaining an identification of the variant allele at the genomic position comprises determining that the respective plurality of nucleic acid fragments support a variant allele call at the genomic position.

For example, in some embodiments, the obtaining an identification of the variant allele at the genomic position is performed by a method that determines, from the plurality of nucleic acid fragments, the likelihood that the genomic position has each genotype in a plurality of candidate genotypes. The selection of a respective genotype from the plurality of candidate genotypes can be determined based on a comparison of the calculated likelihood (e.g., by ranking genotypes by corresponding likelihoods and/or by applying a likelihood threshold to the estimated likelihoods). Generally, the variant allele can be identified as the candidate genotype with the highest likelihood that is not the reference genotype (e.g., the reference allele obtained from a reference genome). In some embodiments, the reference genotype for the genomic position is homozygous (e.g., A/A, T/T, G/G, C/C).

In some embodiments, the obtaining an identification of the variant allele at the genomic position is performed using a Bayesian likelihood model (e.g., variant calling). An example method 320 for variant calling in a test subject can be described with reference to FIG. 3.

Referring to Block 328, in some embodiments, the method 320 for variant calling is performed by deriving the prior probability of a respective genotype at the genomic position (e.g., in electronic format), for each respective candidate genotype in a set of candidate genotypes, using nucleic acid data acquired from a reference population (e.g., a population of a plurality of reference subjects of the given species (e.g., a human)). In some embodiments, the reference population comprises at least one hundred reference subjects. In some embodiments, the reference population comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 reference subjects.

In some embodiments, each respective candidate genotype in the set of genotypes is of the form X/Y, where X is an identity of the base in the set of bases {A, C, T, G} at the genomic position in a reference genome and Y is an identity of the base in the set of bases {A, C, T, G} at the genomic position in the test subject. In other words, in some embodiments, each candidate genotype in the set of genotypes represents a respective diploid genotype, and the paternal and maternal alleles at the genomic position are indicated by X and Y, respectively.

At the single nucleotide level, in some embodiments, there are ten possible genotypes for each autosomal position. In some embodiments, the set of candidate genotypes consists of between two and ten genotypes in the set {A/A, A/C, A/G, A/T, C/C, C/G, C/T, G/G, G/T, and T/T}. In some embodiments, the set of candidate genotypes comprises at least two, there, four, five, six, seven, eight, or nine genotypes in the set {A/A, A/C, A/G, A/T, C/C, C/G, C/T, G/G, G/T, and T/T}. In some embodiments, the set of candidate genotypes consists of the entire set {A/A, A/C, A/G, A/T, C/C, C/G, C/T, G/G, G/T, and T/T}.

Referring to Block 334, in some embodiments, the method 320 for variant calling continues by obtaining, for the genomic position, a strand-specific base count set that comprises a respective forward strand base count and a respective reverse strand base count for each base in the set of {A, T, C, G} at the genomic position, in a forward direction and a reverse direction, which are based on determining (i) a strand orientation and (ii) an identity of a respective base at the genomic position in each respective nucleic acid fragment sequence in the respective plurality of nucleic acid fragment sequences that map to the genomic position. For instance, in some embodiments, the respective plurality of nucleic acid fragment sequences is acquired from a plurality of nucleic acid molecules in a liquid biological sample of the test subject by nucleic acid sequencing and/or methylation sequencing. Details on obtaining the respective plurality of nucleic acid fragment sequences and mapping nucleic acid fragment sequences to a genomic position are further disclosed below, for example, in the section entitled “Obtaining nucleic acid fragment sequences.” In some embodiments, two or more, three or more, four or more, five or more, six or more, 10 or more, 15 or more, 20 or more, 25 or more, 30 or more, 50 or more, or 100 or more nucleic acid fragment sequences map to the genomic position and are accounted for in the strand-specific base count. In some embodiments, bases at the genomic position in the respective plurality of nucleic acid fragment sequences whose identity can be affected by conversion of methylated or unmethylated cytosine do not contribute to the strand-specific base count set.

In some embodiments, the forward direction is a F1R2 read (sense) orientation and the reverse direction is a F2R1 (antisense) read orientation. The pair of orientations can refer to whether a respective nucleic acid fragment sequence originated from a 5′ or 3′ strand of the fragment for a given genomic position. For example, a F1R2 read orientation refers to a sequence read originating from a positive (sense) strand of a nucleic acid fragment, and a F2R1 read orientation refers to a sequence read originating from a negative (antisense) strand of a nucleic acid fragment. In some embodiments, the forward direction is a F1R2 or R2F1 read (sense) orientation and the reverse direction is a F2R1 or R1F2 (antisense) read orientation.

In some embodiments, a strand-specific base count set is used to account for bisulfite conversion. Methylation sequencing can inherently result in strand-specific chemistry that affects the detection of C and T alleles at the genomic position. For instance, bisulfite conversion results in a C to T conversion on the forward strand of a nucleic acid fragment and an A to G conversion on the corresponding reverse strand. Since A and G alleles are not directly affected by bisulfite conversion it can resolve allele counts for the positive strand, where C and T alleles on the positive strand are identified by A and G alleles on the negative strand. As a verification, the total C and T allele count sum can be unaffected by bisulfite conversion.

Referring to Block 340, in some embodiments, the method 320 for variant calling further comprises computing a respective forward strand conditional probability and a respective reverse strand conditional probability for each respective candidate genotype in the set of candidate genotypes for the genomic position using the strand-specific base count set and a sequencing error estimate thereby computing a plurality of forward strand conditional probabilities and a plurality of reverse strand conditional probabilities for the genomic position.

In some embodiments, the sequencing error estimate is between 0.01 and 0.0001. In some embodiments, the sequencing error estimate is less than 0.01, less than 0.009, less than 0.008, less than 0.007, less than 0.006, less than 0.005, less than 0.004, less than 0.003, less than 0.002, less than 0.001, less than 0.00075, less than 0.0005, or less than 0.0075. In some embodiments, a respective sequencing error estimate is used for each candidate genotype in the set of candidate genotypes. In some embodiments, the same sequencing error estimate is used for each candidate genotypes in the set of candidate genotypes. In some embodiments, one or more of the candidate genotypes has a corresponding sequencing error estimate that is distinct from the sequencing error estimate used for the remaining candidate genotypes in the set of candidate genotypes. In some embodiments, symmetric error estimates are assumed for each genotype. In some embodiments, the sequencing error is fixed or variable.

Referring to Block 344, in some embodiments, the method 320 for variant calling further comprises computing a plurality of likelihoods for the genomic position. Each respective likelihood in the plurality of likelihoods is for a respective candidate genotype in the set of candidate genotypes. In some embodiments, the plurality of likelihoods are computed using a combination of (i) the respective forward strand conditional probability for the respective candidate genotype in the plurality of forward strand conditional probabilities, (ii) the respective reverse strand conditional probability for the respective candidate genotype in the plurality of reverse strand conditional probabilities, and (iii) the prior probability of genotype for the respective candidate genotype.

In some embodiments, Bayes' theorem is used to compute the likelihood of observing a respective genotype. In some embodiments, the prior likelihood for each respective genotype is calculated using observed allele frequencies. In some embodiments, each candidate genotype in the set of candidate genotypes for a genomic position is ranked in order of respective Bayesian probability.

In some embodiments, a respective likelihood for a respective candidate genotype in the set of candidate genotypes has the form:

Pr(F_{A},F_{G},F_{CT}|F_{ACGT},genotype,∈)*Pr(R_{AG},R_{C},R_{T}|R_{ACGT},genotype,∈)*Pr(G),

where Pr(F_{A}, F_{G}, F_{CT}|F_{ACGT}, genotype, ∈) is the respective forward strand conditional probability for the respective candidate genotype, Pr(R_{C}, R_{T}, R_{AG} I R_{ACGT}, genotype, ∈) is the respective reverse strand conditional probability for the respective candidate genotype, Pr(G) is the prior probability of genotype at the genomic position for the respective candidate genotype, E is the sequencing error estimate, genotype is the respective candidate genotype, F_{A} is the forward direction base count for base A at the genomic position across the respective plurality of nucleic acid fragment sequences, in the strand-specific base count set, F_{G} is the forward direction base count for base G at the genomic position across the respective plurality of nucleic acid fragment sequences, in the strand-specific base count set, F_{CT} is a summation of (i) the forward direction base count for base C and (ii) the forward direction base count for base T at the genomic position across the respective plurality of nucleic acid fragment sequences, in the strand specific base count set, R_{C} is the reverse direction base count for base C at the genomic position across the respective plurality of nucleic acid fragment sequences, in the strand-specific base count set, R_{T} is the reverse direction base count for base T at the genomic position across the respective plurality of nucleic acid fragment sequences, in the strand-specific base count set, and R_{AG} is a summation of (i) the reverse direction base count for base A and (ii) the reverse direction base count for base G at the genomic position across the respective plurality of nucleic acid fragment sequences, in the strand-specific base count set.

In some embodiments, this multiplication depends on the assumption of symmetric sequencing error estimates for each candidate genome. In some embodiments, the likelihood is a log-likelihood, which is determined by taking the log of the above-defined equation.

In some embodiments, the respective candidate genotype G is A/A and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(A/A),

for A/A comprises calculating:

$$[ {( 1 - ϵ )}^{{F}_{A}} {( \frac{ϵ}{3} )}^{{F}_{G}} {( \frac{2 ϵ}{3} )}^{{F}_{CT}} ] * [ {( 1 - \frac{2 ϵ}{3} )}^{{R}_{AG}} {( \frac{ϵ}{3} )}^{{R}_{C}} {( \frac{ϵ}{3} )}^{{R}_{T}} ] * Pr ( A / A ) .$$

In some embodiments, the respective candidate genotype G is A/A and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(A/C),

for A/A comprises calculating the log-likelihood:

$${log ( 1 - ϵ )}^{{F}_{A}} + {log ( \frac{ϵ}{3} )}^{{F}_{G}} + {log ( \frac{2 ϵ}{3} )}^{{F}_{CT}} + {log ( 1 - \frac{2 ϵ}{3} )}^{{R}_{AG}} + {log ( \frac{ϵ}{3} )}^{{R}_{C}} + {log ( \frac{ϵ}{3} )}^{{R}_{T}} + log ( Pr ( A / A ) ) .$$

In some embodiments, the respective candidate genotype G is A/C and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(A/C),

for A/C comprises calculating:

$$[ {( 0.5 - \frac{ϵ}{3} )}^{{F}_{A}} {( \frac{ϵ}{3} )}^{{F}_{G}} {( 0.5 )}^{{F}_{CT}} ] * [ {( 0.5 )}^{{R}_{AG}} {( 0.5 - \frac{ϵ}{3} )}^{{R}_{C}} {( \frac{ϵ}{3} )}^{{R}_{T}} ] * Pr ( A / C ) .$$

In some embodiments, the respective candidate genotype is G is A/C and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(A/C),

for A/C comprises calculating the log-likelihood:

$${log ( 0.5 - \frac{ϵ}{3} )}^{{F}_{A}} + {log ( \frac{ϵ}{3} )}^{{F}_{G}} + {log ( 0.5 )}^{{F}_{CT}} + {log ( 0.5 )}^{{R}_{AG}} + {log ( 0.5 - \frac{ϵ}{3} )}^{{R}_{C}} + {log ( \frac{ϵ}{3} )}^{{R}_{T}} + log ( Pr ( A / C ) ) .$$

In some embodiments, the respective candidate genotype is G is A/G and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(A/G),

for A/G comprises calculating:

$$[ {( 0.5 - \frac{ϵ}{3} )}^{{F}_{A}} {( 0.5 - \frac{ϵ}{3} )}^{{F}_{G}} {( \frac{2 ϵ}{3} )}^{{F}_{CT}} ] * [ {( 1 - \frac{2 ϵ}{3} )}^{{R}_{AG}} {( \frac{ϵ}{3} )}^{{R}_{C}} {( \frac{ϵ}{3} )}^{{R}_{T}} ] * Pr ( A / G ) .$$

In some embodiments, the respective candidate genotype G is A/G and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(A/G),

for A/G comprises calculating the log-likelihood:

$${log ( 0.5 - \frac{ϵ}{3} )}^{{F}_{A}} + {log ( 0.5 - \frac{ϵ}{3} )}^{{F}_{G}} + {log ( \frac{2 ϵ}{3} )}^{{F}_{CT}} + {log ( 1 - \frac{2 ϵ}{3} )}^{{R}_{AG}} + {log ( \frac{ϵ}{3} )}^{{R}_{C}} + {log ( \frac{ϵ}{3} )}^{{R}_{T}} + log ( Pr ( A / G ) ) .$$

In some embodiments, the respective candidate genotype G is A/T and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(A/T),

for A/T comprises calculating:

$$[ {( 0.5 - \frac{ϵ}{3} )}^{{F}_{A}} {( \frac{ϵ}{3} )}^{{F}_{G}} {( 0.5 )}^{{F}_{CT}} ] * [ {( 0.5 )}^{{R}_{AG}} {( \frac{ϵ}{3} )}^{{R}_{C}} {( 0.5 - \frac{ϵ}{3} )}^{{R}_{T}} ] * Pr ( A / T ) .$$

In some embodiments, the respective candidate genotype G is A/T and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(A/T),

for A/T comprises calculating the log-likelihood:

$${log ( 0.5 - \frac{ϵ}{3} )}^{{F}_{A}} + {log ( \frac{ϵ}{3} )}^{{F}_{G}} + {log ( 0.5 )}^{{F}_{CT}} + {log ( 0.5 )}^{{R}_{AG}} + {log ( \frac{ϵ}{3} )}^{{R}_{C}} + {log ( 0.5 - \frac{ϵ}{3} )}^{{R}_{T}} + log ( Pr ( A / T ) ) .$$

In some embodiments, the respective candidate genotype G is C/C and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(C/C),

for C/C comprises calculating:

$$[ {( \frac{ϵ}{3} )}^{{F}_{A}} {( \frac{ϵ}{3} )}^{{F}_{G}} {( 1 - \frac{2 ϵ}{3} )}^{{F}_{CT}} ] * [ {( \frac{2 ϵ}{3} )}^{{F}_{AG}} {( 1 - ϵ )}^{{R}_{C}} {( \frac{ϵ}{3} )}^{{R}_{T}} ] * Pr ( C / C ) .$$

In some embodiments, the respective candidate genotype G is C/C and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(C/C),

$${log ( \frac{ϵ}{3} )}^{{F}_{A}} + {log ( \frac{ϵ}{3} )}^{{F}_{G}} + {log ( 1 - \frac{2 ϵ}{3} )}^{{F}_{CT}} + {log ( \frac{2 ϵ}{3} )}^{{R}_{AG}} + {log ( 1 - ϵ )}^{{R}_{C}} + {log ( \frac{ϵ}{3} )}^{{R}_{T}} + log ( Pr ( C / C ) ) .$$

In some embodiments, the respective candidate genotype G is C/G and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(C/G),

for C/G comprises calculating:

$$[ {( \frac{ϵ}{3} )}^{{F}_{A}} {( 0.5 - \frac{ϵ}{3} )}^{{F}_{G}} {( 0.5 )}^{{F}_{CT}} ] * [ {( 0.5 )}^{{R}_{AG}} {( 0.5 - \frac{ϵ}{3} )}^{{R}_{C}} {( \frac{ϵ}{3} )}^{{R}_{T}} ] * Pr ( C / G ) .$$

In some embodiments, the respective candidate genotype G is C/G and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(C/G),

for C/G comprises calculating the log-likelihood:

$${log ( \frac{ϵ}{3} )}^{{F}_{A}} + {log ( 0.5 - \frac{ϵ}{3} )}^{{F}_{G}} + {log ( 0.5 )}^{{F}_{CT}} + {log ( 0.5 )}^{{R}_{AG}} + {log ( 0.5 - \frac{ϵ}{3} )}^{{R}_{C}} + {log ( \frac{ϵ}{3} )}^{{R}_{T}} + log ( Pr ( C / G ) ) .$$

In some embodiments, the respective candidate genotype G is C/T and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(C/T),

for C/T comprises calculating:

$$[ {( \frac{ϵ}{3} )}^{{F}_{A}} {( \frac{ϵ}{3} )}^{{F}_{G}} {( 1 - \frac{2 ϵ}{3} )}^{{F}_{CT}} ] * [ {( \frac{2 ϵ}{3} )}^{{R}_{AG}} {( 0.5 - \frac{ϵ}{3} )}^{{R}_{C}} {( 0.5 - \frac{ϵ}{3} )}^{{R}_{T}} ] * Pr ( C / T ) .$$

In some embodiments, the respective candidate genotype G is C/T and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(C/T),

for C/T comprises calculating the log-likelihood:

$${log ( \frac{ϵ}{3} )}^{{F}_{A}} + {log ( \frac{ϵ}{3} )}^{{F}_{G}} + {log ( 1 - \frac{2 ϵ}{3} )}^{{F}_{CT}} + {log ( \frac{2 ϵ}{3} )}^{{R}_{AG}} + {log ( 0.5 - \frac{ϵ}{3} )}^{{R}_{C}} + {log ( 0.5 - \frac{ϵ}{3} )}^{{R}_{T}} + log ( Pr ( C / T ) ) .$$

In some embodiments, the respective candidate genotype G is G/G and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(G/G),

for G/G comprises calculating:

$$[ {( \frac{ϵ}{3} )}^{{F}_{A}} {( 1 - ϵ )}^{{F}_{G}} {( \frac{2 ϵ}{3} )}^{{F}_{CT}} ] * [ {( 1 - \frac{2 ϵ}{3} )}^{{R}_{AG}} {( \frac{ϵ}{3} )}^{{R}_{C}} {( \frac{ϵ}{3} )}^{{R}_{T}} ] * Pr ( G / G ) .$$

In some embodiments, the respective candidate genotype G is G/G and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(G/G),

for G/G comprises calculating the log-likelihood:

$${log ( \frac{ϵ}{3} )}^{{F}_{A}} + {log ( 1 - ϵ )}^{{F}_{G}} + {log ( \frac{2 ϵ}{3} )}^{{F}_{CT}} + {log ( 1 - \frac{2 ϵ}{3} )}^{{R}_{AG}} + {log ( \frac{ϵ}{3} )}^{{R}_{C}} + {log ( \frac{ϵ}{3} )}^{{R}_{T}} + log ( Pr ( G / G ) ) .$$

In some embodiments, the respective candidate genotype G is G/T and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(G/T),

for G/T comprises calculating:

$$[ {( \frac{ϵ}{3} )}^{{F}_{A}} {( 0.5 - \frac{ϵ}{3} )}^{{F}_{G}} {( 0.5 )}^{{F}_{CT}} ] * [ {( 0.5 )}^{{R}_{AG}} {( \frac{ϵ}{3} )}^{{R}_{C}} {( 0.5 - \frac{ϵ}{3} )}^{{R}_{T}} ] * Pr ( G / T ) .$$

In some embodiments, the respective candidate genotype G is G/T and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(G),

for G/T comprises calculating the log-likelihood:

$${log ( \frac{ϵ}{3} )}^{{F}_{A}} + {log ( 0.5 - \frac{ϵ}{3} )}^{{F}_{G}} + {log ( 0.5 )}^{{F}_{CT}} + {log ( 0.5 )}^{{R}_{AG}} + {log ( \frac{ϵ}{3} )}^{{R}_{C}} + {log ( 0.5 - \frac{ϵ}{3} )}^{{R}_{T}} + log ( Pr ( G / T ) ) .$$

In some embodiments, the respective candidate genotype G is T/T and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(T/T),

for T/T comprises calculating:

$$[ {( \frac{ϵ}{3} )}^{{F}_{A}} {( \frac{ϵ}{3} )}^{{F}_{G}} {( 1 - \frac{2 ϵ}{3} )}^{{F}_{CT}} ] * [ {( \frac{2 ϵ}{3} )}^{{R}_{AG}} {( \frac{ϵ}{3} )}^{{R}_{C}} {( 1 - ϵ )}^{{R}_{T}} ] * Pr ( T / T ) .$$

In some embodiments, the respective candidate genotype G is T/T and computing the respective likelihood:

Pr(F_{A},F_{G},F_{CT}|F_{ACGT},genotype,∈)*Pr(R_{AG},R_{C},R_{T}|R_{ACGT},genotype,∈)*Pr(T/T),

for T/T comprises calculating the log-likelihood:

$${log ( \frac{ϵ}{3} )}^{{F}_{A}} + {log ( \frac{ϵ}{3} )}^{{F}_{G}} + {log ( 1 - \frac{2 ϵ}{3} )}^{{F}_{CT}} + {log ( \frac{2 ϵ}{3} )}^{{R}_{AG}} + {log ( \frac{ϵ}{3} )}^{{R}_{C}} + {log ( ( 1 - ϵ ) )}^{{R}_{T}} + log ( Pr ( T / T ) ) .$$

In some embodiments, one or more respective likelihood calculations further includes a corresponding bisulfate-conversion-rate prior to account for apparent disparities between the counts of C on corresponding forward and reverse strands. For example, if a higher number of C bases are observed on a forward strand, that would suggest that a T/T is ultimately less likely than a C/T of C/C genotype. Examples of likelihood calculations that account for bisulfate conversion rates, base quality scores, and other sequencing information are known in the art.

Referring to Block 346, in some embodiments, the method 320 for variant calling further comprises determining whether the plurality of likelihoods (e.g., computed in Block 344) supports a variant call at the genomic position. In some embodiments, this comprises determining whether any likelihood in the plurality of likelihoods for any of the proposed genotypes (including, e.g., the reference genotype) for the genomic position satisfies a variant threshold. In some embodiments, when a likelihood for any of the proposed genotypes (including, e.g., the reference genotype) for the genomic position satisfies a variant threshold, a variant at the genomic position is deemed identified. Thus, from among the plurality of likelihoods corresponding to a plurality of different variant alleles, a variant allele is called from among the plurality of different variant alleles if the likelihood for the variant allele satisfies a threshold value. If more than two variant alleles satisfy the threshold value, then the variant allele with the greatest likelihood satisfying the threshold is called. If none of the variant alleles satisfies the threshold value, no variant allele is called.

In some embodiments, the likelihood is expressed as a log-likelihood (e.g., an unnormalized likelihood) and the variant threshold is satisfied when the log-likelihood for the reference genotype for the genomic position is less than −10. In some embodiments, a variant threshold is satisfied when the log-likelihood for the reference genotype for the genomic position is less than −1, less than −5, less than −10, less than −25, less than −50, or less than −100. In some embodiments, the likelihood is expressed as a log-likelihood and the variant threshold is satisfied when the log-likelihood for the reference genotype for the genomic position is between −25 and −5. In some embodiments, the likelihood is expressed as a log-likelihood and the variant threshold is satisfied when the log-likelihood for the reference genotype for the genomic position is between −10 and −1, between −10 and −5, between −25 and −1, between −25 and −10, between −25 and −15, between −50 and −1, between −50 and −5, between −50 and −10, or between −50 and −25.

In some embodiments, the method 320 further comprises determining, when a variant at the genomic position is called, an identity of the variant by selecting the candidate genotype in the set of candidate genotypes for the genomic position that has the best likelihood in the plurality of likelihoods as the variant. In some embodiments, this determination can rank the candidate genotypes by their corresponding likelihoods or log-likelihoods. In some embodiments, a single identity for the variant is called, by selecting the top ranked genotype for the variant. In some embodiments, at least 2, at least 3, or at least 4 identities for the variant are called, by selecting the top 2, the top 3, or the top 4 best ranked genotypes for the variant, respectively.

In some embodiments, the method 320 further comprises repeating the method for each genomic position in a plurality of genomic positions for the test subject (e.g., thereby obtaining a plurality of variant calls for the test subject).

In some embodiments, the plurality of variant calls comprises 200 variant calls. In some embodiments, the plurality of variant calls comprises at least 10 variant calls, at least 20 variant calls, at least 30 variant calls, at least 40 variant calls, at least 50 variant calls, at least 60 variant calls, at least 70 variant calls, at least 80 variant calls, at least 90 variant calls, at least 100 variant calls, at least 200 variant calls, at least 300 variant calls, at least 400 variant calls, at least 500 variant calls, at least 600 variant calls, at least 700 variant calls, at least 800 variant calls, at least 900 variant calls, at least 1000 variant calls, at least 2000 variant calls, at least 3000 variant calls, at least 4000 variant calls, between 10 and 10,000 variant calls, between 50 and 5000 variant calls or between 100 and 4500 variant calls for the test subject using the sequencing data obtained from the biological sample of the test subject. In some embodiments, the number of variant calls obtained in the plurality of variant calls corresponds to the number of genomic positions in the plurality of genomic positions.

In some embodiments, the plurality of variant calls is filtered. For example, in some embodiments, a variant call obtained using any of the methods disclosed herein fails to satisfy one or more filtering criteria, and is not retained for further analysis (e.g., for identifying the variant allele as somatic or germline).

In some embodiments, a variant call is removed from further analysis if is determined to be a germline variant call using a sequencing dataset obtained from a matched germline sample from the test subject. For example, in some embodiments, the method further comprises obtaining a second plurality of variant calls using a second plurality of nucleic acid fragment sequences, in electronic form, acquired from a sequencing of a second plurality of nucleic acid fragments in a second biological sample of the test subject, where the second biological sample is a matched germline sample from the subject (e.g., a normal tissue sample), and removing each respective variant call from the plurality of variant calls that is also in the second plurality of variant calls (e.g., removing germline variant calls). In some embodiments, a variant allele is identified as a germline variant when a variant caller algorithm, such as FreeBayes, VarDict, MuTect, MuTect2, MuSE, FreeBayes, VarDict, and/or MuTect identifies the variant as a germline variant (e.g., for a test subject using a sample-matched sequencing assay).

In some embodiments, a variant call is removed from further analysis if it is a germline variant call obtained from a list of known germline variants (e.g., gnomad, dbSNP). GnomAD and dbSNP refer to reference databases of known germline variants. In some embodiments, any other known germline variants are removed from the first plurality of variant calls.

In some embodiments, a variant call is removed from further analysis if it has been found in a tissue sample of a subject other than the test subject (e.g., a recurrent variant tissue blacklist). For example, in some embodiments, certain portions of a reference genome are determined to have higher information value (e.g., to be more informative in determining variants or in downstream analysis).

In some embodiments, a variant call is removed from further analysis if it fails to satisfy a quality metric (e.g., minimum allele fraction, maximum allele fraction, quality of base calls (e.g., Phred scores), minimum depth, etc.).

In some embodiments, the quality metric is a minimum variant allele fraction in the respective plurality of nucleic acid fragment sequences, in electronic form, that map to the genomic position of the respective variant call. In some embodiments, the minimum variant allele fraction is ten percent. In some embodiments, the minimum variant allele fraction is less than 1 percent, less than 2 percent, less than 3 percent, less than 4 percent, less than 5 percent, less than 6 percent, less than 7 percent, less than 8 percent, less than 9 percent, less than 10 percent less than 15 percent, or less than 20 percent.

In some embodiments, the quality metric is a maximum variant allele fraction in the respective plurality of nucleic acid fragment sequences, in electronic form, that map to the genomic position of the respective variant call. In some embodiments, the maximum variant allele fraction is ninety percent. In some embodiments, the maximum variant allele fraction is at least 55 percent, at least 60 percent, at least 70 percent, at least 80 percent, at least 90 percent, at least 95 percent, or at least 99 percent.

In some embodiments, the quality metric is a minimum depth in the respective plurality of nucleic acid fragment sequences, in electronic form, that map to the genomic position of the respective variant call. In some embodiments, the minimum depth is ten. In some embodiments, the minimum depth is at least 5, at least 10, at least 50, at least 100, or at least 200.

In some embodiments, a variant call is removed from further analysis if it is listed in a blacklist of known noisy genomic positions. In some embodiments, such sites are based on a set of 642 samples from the CCGA-1 method, described below in Example 5. In some embodiments, the blacklist is all or a portion of the ENCODE blacklist.

In some embodiments, variant calling is performed using a matched normal control sample (e.g., using cfDNA from a liquid biological sample and a patient-matched normal tissue sample). In some embodiments, variant calling is performed without a matched normal control sample (e.g., using cfDNA from a liquid biological sample).

Alternate methods for variant calling can be contemplated. Suitable variant calling methods include methods for calling SNVs and indels (e.g., FreeBayes, GATK HaplotypeCaller, Platypus, Samtools/BCFtools, etc.), methods for calling somatic mutations (e.g., deepSNV, MuSE, MuTect2, SomaticSniper, Strelka2, VarDict, VarScan2, etc.), methods for calling copy number variants (e.g., cn.MOPS, CONTRA, CoNVEX, ExomeCNV, ExomeDepth, XHMM, etc.), methods for calling structural variants (e.g., DELLY, Lumpy, Manta, Pindel, SVMerge, etc.), and/or methods for calling gene fusions (RNA-seq) (e.g., fusionCatcher, fusionMap, mapSplice, SOAPfuse, STAR-Fusion, TopHat-Fusion, etc.) In some embodiments, variant calling is performed using any of the methods disclosed herein, or any substitutions, modifications, additions, deletions, and/or combinations thereof.

Methods for variant calling are described in greater detail in U.S. patent application Ser. No. 17/185,885, filed Feb. 25, 2021, entitled “Systems and Methods for Calling Variants using Methylation Sequencing Data,” and PCT Application No. PCT/US2021/019746, filed February 2021, entitled “Systems and Methods for Calling Variants using Methylation Sequencing Data,” each of which is hereby incorporated herein by reference in its entirety.

Obtaining Nucleic Acid Fragment Sequences.

Referring to Block 206 of FIG. 2A, the method further comprises obtaining a methylation state and a respective sequence of each nucleic acid fragment sequence in a respective plurality of nucleic acid fragment sequences in a sequencing dataset (e.g., comprising at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷ or at least 1×10⁸ nucleic acid fragment sequences) derived from a biological sample (e.g., a liquid biological sample) obtained from the test subject that map onto the genomic position.

In some embodiments, the biological sample is prepared for sequencing using any suitable method (see, above, “Subjects and samples”). In some embodiments, the preparation of the biological sample comprises obtaining a respective plurality of nucleic acid fragments (e.g., nucleic acid molecules) for the test subject. In some embodiments, the respective plurality of nucleic acid fragments obtained from the biological sample are cell-free nucleic acid fragments.

After obtaining a plurality of nucleic acid fragments from a biological sample, in some embodiments, the nucleic acid fragments are sequenced. In some embodiments, the sequencing is methylation sequencing. In some embodiments, the methylation sequencing is whole-genome methylation sequencing. In some embodiments, the methylation sequencing is targeted DNA methylation sequencing using a plurality of nucleic acid probes. In some embodiments, the plurality of nucleic acid probes comprises one hundred or more probes. In some embodiments, the plurality of nucleic acid probes comprises 100 or more, 200 or more, 300 or more, 400 or more, 500 or more, 600 or more, 700 or more, 800 or more, 900 or more, 1000 or more, 2000 or more, 3000 or more, 4000 or more 5000 or more, 6000 or more, 7000 or more, 8000 or more, 9000 or more, 10,000 or more, 25,000 or more, or 50,000 or more probes. In some embodiments, the plurality of nucleic acid probes comprises no more than 50,000, no more than 250,000, no more than 10,000, no more than 9000, no more than 8000, no more than 7000, no more than 6000, no more than 5000, no more than 4000, no more than 3000, no more than 2000, no more than 1000, no more than 900, no more than 800, no more than 700, no more than 600, or no more than 500 probes. In some embodiments, the plurality of nucleic acid probes comprises from 100 to 500, from 500 to 1000, from 1000 to 2000, from 1000 to 5000, from 100 to 5000, from 5000 to 10,000, or from 10,000 to 50,000 probes. In some embodiments, the plurality of nucleic acid probes falls within another range starting no lower than 100 probes and ending no higher than 50,000 probes. In some embodiments, some or all of the probes uniquely map to a genomic region described in International Patent Publication No. WO2020154682A3, entitled “Detecting Cancer, Cancer Tissue or Origin, or Cancer Type,” which is hereby incorporated by reference, including the Sequence Listing referenced therein. In some embodiments, some or all of the probes uniquely map to a genomic region described in International Patent Publication No. WO2020/069350A1, entitled “Methylated Markers and Targeted Methylation Probe Panel,” which is hereby incorporated by reference, including the Sequence Listing referenced therein. In some embodiments, some or all of the probes uniquely map to a genomic region described in International Patent Publication No. WO2019/195268A2, entitled “Methylated Markers and Targeted Methylation Probe Panels,” which is hereby incorporated by reference, including the Sequence Listing referenced therein.

In some embodiments, the methylation sequencing detects one or more 5-methylcytosine (5mC) and/or 5-hydroxymethylcytosine (5hmC) in respective nucleic acid fragments in the respective plurality of nucleic acid fragments. In some embodiments, the methylation sequencing comprises conversion of one or more unmethylated cytosines or one or more methylated cytosines, in the nucleic acid fragments in the respective plurality of nucleic acid fragments, to a corresponding one or more uracils. In some embodiments, the one or more uracils are converted during amplification and detected during the methylation sequencing as one or more corresponding thymines. In some embodiments, the conversion of one or more unmethylated cytosines or one or more methylated cytosines comprises a chemical conversion, an enzymatic conversion, or combinations thereof.

In some embodiments, prior to sequencing, the plurality of nucleic acid fragments is treated to convert unmethylated cytosines to uracils. In some embodiments, the methylation sequencing is bisulfite sequencing. For instance, in some embodiments, the method uses a bisulfite treatment of DNA (e.g., cfDNA) that converts the unmethylated cytosines to uracils without converting the methylated cytosines. For example, a commercial kit such as the EZ DNA Methylation™—Gold, EZ DNA Methylation™—Direct, or EZ DNA Methylation™—Lightning kit (available from Zymo Research Corp (Irvine, Calif.)) is used for the bisulfite conversion in some embodiments. In some embodiments, the conversion of unmethylated cytosines to uracils is accomplished using an enzymatic reaction. For example, the conversion can use a commercially available kit for the conversion of unmethylated cytosines to uracils, such as APOBEC-Seq (NEBiolabs, Ipswich, Mass.).

In some embodiments, the methylation sequencing is whole genome bisulfite sequencing. In some embodiments, the whole-genome bisulfite sequencing assay looks for variations in methylation patterns in the genome. See, United States Patent Publication No. US 2019-0287652 A1, entitled “Anomalous Fragment Detection and Classification,” which is hereby incorporated by reference.

From the converted cell-free nucleic acid fragments, a sequencing library is prepared. Optionally, the sequencing library is enriched for cell-free nucleic acid fragments, or genomic regions, that are informative for cell origin using a plurality of hybridization probes, such as any combination of regions disclosed in, for example, International Patent Publication No. WO2020154682A3, entitled “Detecting Cancer, Cancer Tissue or Origin, or Cancer Type,” International Patent Publication No. WO2020/069350A1, entitled “Methylated Markers and Targeted Methylation Probe Panel,” and/or International Patent Publication No. WO2019/195268A2, entitled “Methylated Markers and Targeted Methylation Probe Panels,” each of which is hereby incorporated by reference. In some embodiments, the hybridization probes are short oligonucleotides that hybridize to particularly specified cell-free nucleic acid fragments, or targeted regions, and enrich for those fragments or regions for subsequent sequencing and analysis as disclosed in for example, International Patent Publication No. WO2020154682A3, entitled “Detecting Cancer, Cancer Tissue or Origin, or Cancer Type,” International Patent Publication No. WO2020/069350A1, entitled “Methylated Markers and Targeted Methylation Probe Panel,” and/or International Patent Publication No. WO2019/195268A2, entitled “Methylated Markers and Targeted Methylation Probe Panels,” each of which is hereby incorporated by reference. In some embodiments, hybridization probes are used to perform targeted, high-depth analysis of a set of specified CpG sites that are informative for cell origin. Once prepared, the sequencing library or a portion thereof can be sequenced to obtain a plurality of sequence reads (e.g., nucleic acid fragment sequences).

In some embodiments, any form of sequencing can be used to obtain sequence reads (e.g., nucleic acid fragment sequences) from the plurality of nucleic acid fragments derived from the biological sample of the test subject. Example sequencing methods include, but are not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain sequence reads from the plurality of nucleic acid fragments obtained from the biological sample.

In some embodiments, sequencing-by-synthesis and reversible terminator-based sequencing (e.g., Illumina's Genome Analyzer; Genome Analyzer II; HISEQ 2000; HISEQ 2500 (Illumina, San Diego Calif.)) is used to obtain sequence reads from the plurality of nucleic acid fragments (e.g., cell-free nucleic acid fragments) from the biological sample. In some such embodiments, millions of nucleic acid fragments (e.g., cfDNA fragments) are sequenced in parallel. In one example of this type of sequencing technology, a flow cell is used that contains an optically transparent slide with eight individual lanes on the surfaces of which are bound oligonucleotide anchors (e.g., adaptor primers). A flow cell often is a solid support that is configured to retain and/or allow the orderly passage of reagent solutions over bound analytes. In some instances, flow cells are planar in shape, optically transparent, generally in the millimeter or sub-millimeter scale, and often have channels or lanes in which the analyte/reagent interaction occurs. In some embodiments, a sample comprising a plurality of nucleic acid fragments (e.g., cfDNA fragments) can include a signal or tag that facilitates detection. In some such embodiments, the acquisition of sequence reads from the nucleic acid fragments includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

In some embodiments, the sequencing comprises whole genome methylation sequencing (e.g., whole genome bisulfite sequencing (WGBS)) and/or whole genome sequencing (e.g., whole genome sequencing (WGS) or whole exome sequencing (WES)), and the sequencing is used to sequence at least a portion of the genome of the test subject. In some embodiments, the portion of the genome is at least 10 percent, 20 percent, 30 percent, 40 percent, 50 percent, 60 percent, 70 percent, 80 percent, 90 percent, 95 percent, 99 percent, 99.9 percent or all of a genome (e.g., a human reference genome). In some embodiments, the sequencing comprises whole genome methylation sequencing and/or whole genome sequencing, and the sequencing obtains a sequencing coverage (e.g., sequencing depth) of the portion of the genome that is at least lx, at least 2×, at least 3×, at least 4×, at least 5×, at least 10×, at least 15×, at least 20×, at least 25×, at least 30×, at least 50×, at least 100×, at least 200×, at least 300×, at least 400×, at least 500×, or at least 1000× across the sequenced portion of the genome. In some embodiments, the sequencing obtains a sequencing coverage of at least 5×, at least 10×, at least 15×, at least 20×, at least 25×, at least 30×, at least 50×, at least 100×, at least 200×, at least 300×, at least 400×, at least 500×, or at least 1000× across the entire genome.

In some embodiments, the sequencing is a targeted sequencing (e.g., a targeted methylation sequencing), and the targeted sequencing obtains a sequencing coverage (e.g., sequencing depth) of at least 5×, at least 10×, at least 15×, at least 20×, at least 25×, at least 30×, at least 50×, at least 100×, at least 250×, at least 500×, or at least 1000× of the targeted portions of the genome of the test subject (e.g., a panel of genes to which one or more probes map). In some embodiments, the targeted sequencing obtains a sequencing coverage of at least 100×, at least 200×, at least 500×, at least 1,000×, at least 2,000×, at least 3,000×, at least 4,000×, at least 5,000×, at least 10,000×, at least 15,000×, at least 20,000×, at least 25,000×, at least 30,000×, at least 40,000×, at least 50,000×, at least 60,000×, or at least 70,000× across the targeted regions of the genome.

In some embodiments, the plurality of sequence reads (e.g., nucleic acid fragment sequences) obtained from the sequencing of the biological sample comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads in a sequencing dataset. In some embodiments, the plurality of sequence reads comprises at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, or more sequence reads in a sequencing dataset. In some embodiments, the plurality of sequence reads comprises no more than 5×10⁷, no more than 1×10⁷, no more than 5×10⁶, no more than 4×10⁶, no more than 3×10⁶, no more than 2×10⁶, no more than 1×10⁶, no more than 500,000, no more than 100,000, no more than 50,000, no more than 30,000, no more than 20,000, no more than 10,000, no more than 9000, no more than 8000, no more than 7000, no more than 6000, no more than 5000, no more than 4000, no more than 3000, no more than 2000, no more than 1000, or less sequence reads in a sequencing dataset. In some embodiments, the plurality of sequence reads comprises from 1000 to 5000, from 1000 to 10,000, from 2000 to 20,000, from 5000 to 50,000, from 10,000 to 100,000, from 100,000 to 500,000 from 10,000 to 500,000, from 500,000 to 1 million, from 1 million to 30 million, from 30 million to 80 million, or from 10 million to 500 million sequence reads in a sequencing dataset. In some embodiments, the plurality of sequence reads falls within another range starting no lower than 1000 sequence reads and ending no higher than 1×10⁹ sequence reads.

In some embodiments, the obtaining the respective sequence of each nucleic acid fragment sequence in the plurality of nucleic acid fragment sequences further comprises mapping each nucleic acid fragment sequence in the sequencing dataset to a reference sequence (e.g., a human reference genome). In some embodiments, the method comprises mapping, to the reference sequence, all or a portion of the sequencing dataset comprising the plurality of nucleic acid fragment sequences.

For instance, for a respective genomic position, in some embodiments, the method further comprises inputting a reference genome (e.g., a human reference genome) into a computer system comprising a processor coupled to a non-transitory memory, and using the computer system to determine that each respective nucleic acid fragment sequence in the respective plurality of nucleic acid fragment sequences maps to the genomic position by aligning the respective nucleic acid fragment sequence to the reference genome.

In some embodiments, mapping is performed using a Smith-Waterman gapped alignment as implemented in, for example, Arioc, or a Burrows-Wheeler transform as implemented in, for example, Bowtie. Other suitable alignment programs can include, but are not limited to, BarraCUDA, BBMap, BFAST, BigBWA, BLASTN, BLAT, BWA, BWA-PS SM, CASHX. In some embodiments, the mapping allows mismatching. In some embodiments, the mapping comprises at least 1, at least 2, at least 3, at least 4, at least 5, at least 6, at least 7, at least 8, at least 9, at least 10, or more than 10 mismatches. Other methods of mapping sequence reads to a reference sequence can be used.

In some embodiments, mapping a nucleic acid fragment sequence in the sequencing dataset to a reference sequence comprises using a CpG index. For example, in some embodiments, a CpG index comprises a list of each CpG site in the plurality of CpG sites (e.g., CpG 1, CpG 2, CpG 3, etc.) in a reference sequence (e.g., a human reference genome). The CpG index can further comprise a corresponding genomic location, in the corresponding reference sequence, for each respective CpG site in the CpG index. Each CpG site in each respective nucleic acid sequence fragment can thus be indexed to a specific location in the respective reference sequence, which can be determined using the CpG index. In some embodiments, a reference sequence is obtained in electronic format.

In some embodiments, for a respective genomic position, the method comprises mapping all or a portion of the sequencing dataset comprising the plurality of nucleic acid fragment sequences to at least the portion of the reference sequence containing the genomic position.

In some embodiments, each nucleic acid fragment sequence in the plurality of nucleic acid fragment sequences that map to the genomic position is determined, by the mapping, to overlap all or part of the genomic position.

In some embodiments, the plurality of nucleic acid fragment sequences that map to the genomic position comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 10,000, at least 20,000, or at least 30,000 nucleic acid fragment sequences that map to the genomic position. In some embodiments, the plurality of nucleic acid fragment sequences that map to the genomic position comprises no more than 70,000, no more than 50,000, no more than 30,000, no more than 10,000, no more than 5000, no more than 2000, no more than 1000, no more than 900, no more than 800, no more than 700, no more than 600, no more than 500, no more than 400, no more than 300, no more than 200, no more than 100, no more than 50, or no more than 30 nucleic acid fragment sequences that map to the genomic position. In some embodiments, the plurality of nucleic acid fragment sequences that map to the genomic position comprises from 5 to 20, from 20 to 50, from 50 to 100, from 100 to 500, from 500 to 1000, from 500 to 5000, from 2000 to 10,000, or from 10,000 to 70,000 nucleic acid fragment sequences that map to the genomic position. In some embodiments, the plurality of nucleic acid fragment sequences that map to the genomic position falls within another range starting no lower than 10 nucleic acid fragment sequences and ending no higher than 70,000 nucleic acid fragment sequences. In some embodiments, the plurality of nucleic acid fragment sequences that map to the genomic position is determined at least in part based on the sequencing coverage (e.g., sequencing depth) of the sequencing method used.

In some embodiments, where the method is performed for each of a plurality of genomic positions, the mapping comprises mapping the plurality of nucleic acid fragment sequences to at least the regions of the reference sequence (e.g., reference genome) containing the plurality of genomic positions.

In some embodiments, the obtaining the methylation state of each respective nucleic acid fragment sequence in the sequencing dataset comprises determining a corresponding methylation state for each respective CpG site in the respective nucleic acid fragment sequence. For instance, in some embodiments, a respective nucleic acid fragment sequence can have one or more CpG sites, and each respective CpG site in the nucleic acid fragment sequence is determined by the methylation sequencing to have a corresponding methylation state.

In some embodiments, the methylation state of a respective CpG site in the corresponding one or more CpG sites in the respective nucleic acid fragment sequence is methylated when the respective CpG site is determined by the methylation sequencing to be methylated and unmethylated when the respective CpG site is determined by the methylation sequencing to not be methylated. In some embodiments, a methylated state is represented as “M”, and an unmethylated state is represented as “U”.

Other methylation states can be possible. For example, in some embodiments, the methylation state is “other” when the methylation sequencing is unable to call the methylation state of the respective CpG site as methylation or unmethylated. In some embodiments, possible methylation states further include but are not limited to ambiguous (e.g., meaning the underlying CpG is not covered by any fragment sequences in the plurality of fragment sequences), variant (e.g., meaning that the fragment sequence is not consistent with a CpG occurring in its expected position based on a reference sequence and can be caused by a real variant at the site or a sequence error), or conflict (e.g., when two or more fragment sequences both overlap a CpG site but have inconsistent methylation states). See, e.g., U.S. Provisional Patent Application 62/948,129, entitled “Cancer classification using patch convolutional neural networks,” filed Dec. 13, 2019, which is hereby incorporated herein by reference in its entirety.

In some embodiments, the obtaining the methylation state of each respective nucleic acid fragment sequence in the sequencing dataset comprises determining a methylation state vector for the nucleic acid fragment sequence. In some embodiments, a methylation state vector is a sequence of methylation states indicating the methylation states of all CpG sites contained in the respective nucleic acid fragment. Methylation state vectors are further described, for example, in U.S. patent application Ser. No. 16/352,602, entitled “Anomalous Fragment Detection and Classification,” filed Mar. 13, 2019, or in accordance with any of the techniques disclosed in U.S. Provisional Patent Application No. 62/847,223, entitled “Model-Based Featurization and Classification,” filed May 13, 2019, each of which is hereby incorporated by reference.

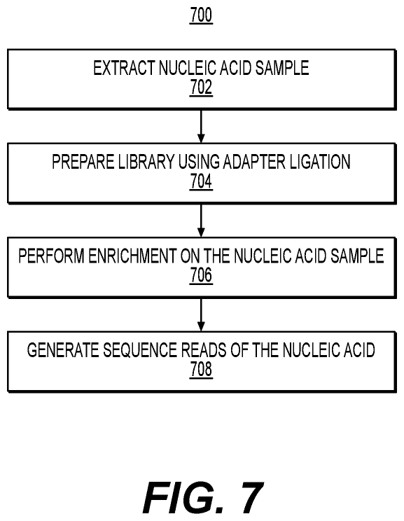

Sequencing methods for nucleic acid fragments obtained from a biological sample of a test subject, including processing biological samples, extracting nucleic acid fragments from biological samples, treatment of nucleic acid fragments for methylation sequencing, preparation of sequencing libraries, enrichment of target nucleic acids, hybridization probes, obtaining sequence reads, mapping fragment sequences to a reference sequence, and/or generation of methylation state vectors, are further described in detail in Examples 1, 2, and 4, below, with reference to FIGS. 7, 8, and 9. Other methods for obtaining nucleic acid fragment sequences, including processing biological samples, extracting nucleic acid fragments from biological samples, treatment of nucleic acid fragments for methylation sequencing, preparation of sequencing libraries, enrichment of target nucleic acids, hybridization probes, obtaining sequence reads, mapping fragment sequences to a reference sequence, and/or generation of methylation state vectors, are contemplated.

Assigning Subsets.

Referring to Block 208, the method further comprises using (i) the identification of the reference allele at the genomic position and (ii) the respective sequence of each nucleic acid fragment sequence in the respective plurality of nucleic acid fragment sequences to assign each nucleic acid fragment sequence in the respective plurality of nucleic acid fragment sequences that has the reference allele, at the genomic position, to a reference subset. The method also includes using (i) the identification of the variant allele at the genomic position and (ii) the respective sequence of each nucleic acid fragment sequence in the respective plurality of nucleic acid fragment sequences to assign each nucleic acid fragment sequence in the respective plurality of nucleic acid fragment sequences that has the variant allele, at the genomic position, to a variant subset.

In some embodiments, the assignment of each nucleic acid fragment sequence to the reference subset comprises determining, for each respective nucleic acid fragment sequencing in the sequencing dataset, whether the respective nucleic acid fragment sequence has the reference allele at the genomic position, based on a comparison between the nucleic acid fragment sequence obtained by sequencing and the nucleic acid sequence of the reference allele (identified as described above with reference to Block 202; see, “Reference and variant alleles”). In some embodiments, the comparison is performed using a look-up table.

In some embodiments, the assignment of each nucleic acid fragment sequence to the variant subset comprises determining, for each respective nucleic acid fragment sequencing in the sequencing dataset, whether the respective nucleic acid fragment sequence has the variant allele at the genomic position, based on a comparison between the nucleic acid fragment sequence obtained by sequencing and the nucleic acid sequence of the variant allele (identified as described above with reference to Block 204; see, “Reference and variant alleles”).

In some embodiments, the method comprises obtaining a count of the number of nucleic acid fragment sequences assigned to the reference subset.

In some embodiments, the method comprises obtaining a count of the number of nucleic acid fragment sequences assigned to the variant subset.

In some embodiments, the plurality of nucleic acid fragment sequences in the sequencing dataset is filtered using one or more filters. In some embodiments, the filtering occurs prior to the assignment of nucleic acid fragment sequences to a reference subset and a variant subset. In some embodiments, the filtering occurs after the assignment of nucleic acid fragment sequences to a reference subset and a variant subset. In some embodiments, the filtering is performed using the counts of the nucleic acid fragment sequences assigned to the reference and variant subsets. In some embodiments, the filtering comprises removing one or more nucleic acid fragment sequences that fail to satisfy a filtering criterion from the respective plurality of nucleic acid fragment sequences for a respective genomic position. In some embodiments, where the method is performed for a plurality of genomic positions, the filtering comprises removing one or more genomic positions that fail to satisfy a filtering criterion from the plurality of genomic positions. In some embodiments, where the method is performed for a plurality of genomic positions, the filtering comprises removing a genomic position from the plurality of genomic positions, when at least a threshold amount of nucleic acid fragment sequences that map to the respective genomic position fail to satisfy a filtering criterion.

For example, in some embodiments, the plurality of nucleic acid fragment sequences in the sequencing dataset is filtered based on a ratio of fragments containing the variant allele to fragments containing the reference allele at the genomic position. In some embodiments, where the method is performed for a plurality of genomic positions, the filtering comprises removing genomic positions that have less than a threshold ratio of variant allele fragments to reference allele fragments. In some embodiments, where the method is performed for a plurality of genomic positions, the filtering comprises removing genomic positions that have less than a threshold count of variant allele fragments in the variant subset.

In some embodiments, the threshold count of variant allele fragments in the variant subset is at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, at least 500, or at least 1000 nucleic acid fragments from the test subject that map to the genomic region of the variant allele and have the variant allele.

In some embodiments, the one or more filters comprise a minimum variant allele frequency, a maximum variant allele frequency, a minimum sequencing depth for a respective allele, a blacklist of germline variants from the test subject (e.g., as marked by freebayes), a blacklist of a custom database (e.g., a recurrent tissue blacklist), or a blacklist of germline variants from a reference database (e.g., from the gnomad and/or dbSNP databases.

In some embodiments, the one or more filters is a minimum variant allele frequency (minimum VAF). In some such embodiments, the minimum allele frequency is at least 3%, at least 5%, at least 10%, at least 15%, at least 20%, at least 25%, at least 30%, at least 35%, at least 40%, at least 45%, or at least 50% of the nucleic acid fragments from the test subject.

In some embodiments, the one or more filters is a maximum variant allele frequency (maximum VAF). In some embodiments, the maximum allele frequency is 95% or less, 90% or less, 85% or less, 80% or less, 75% or less, 70% or less, 65% or less, 60% or less, 55% or less, or 50% or less of the nucleic acid fragments from the test subject.

In some embodiments, the one or more filters is a minimum sequencing depth (e.g., for all nucleic acid fragment sequences at the genomic position, including the reference subset and the variant subset). In some embodiments, the minimum sequencing depth is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, at least 500, or at least 1000 nucleic acid fragments from the test subject that map to the genomic position.

Other filters can be contemplated. For instance, in some embodiments, the plurality of nucleic acid fragment sequences is filtered, e.g., for depth, minimum mapping quality (MAPQ), duplicate fragments, uncalled fragments, unconverted fragments, ambiguous calls, variant calls, conflicted calls, minimum or maximum fragment length, minimum or maximum number of base pairs, minimum or maximum CpG count, and/or p-value (described in greater detail below).

Additionally, in some embodiments, the sequencing dataset is further processed by any suitable method, such as by a bioinformatics pipeline. For instance, in some embodiments, the plurality of nucleic acid fragment sequences is further normalized, e.g., to account for pull-down, amplification, background copy number (e.g., duplication), and/or sequencing bias (e.g., mappability, GC bias etc.).

Input indications.

Referring to Block 210, the method further comprises applying, to a trained binary classifier (e.g., comprising at least 10 parameters), at least (i) one or more indications of methylation state across the methylation state of each nucleic acid fragment sequence in the variant subset and (ii) an indication of a number of nucleic acid fragment sequences in the reference subset versus a number of nucleic acid fragment sequences in the variant subset, thereby obtaining from the trained binary classifier an identification of the variant allele at the genomic position in the test subject as somatic or germline.

In some embodiments, the (i) one or more indications of methylation state across the methylation state of each nucleic acid fragment in the variant subset is a p-value. In some embodiments, the p-value indicates whether the respective nucleic acid fragment is anomalously methylated relative to a healthy reference.

Thus, referring to Block 212 of FIG. 2B, in an example embodiment, a first nucleic acid fragment sequence in the respective plurality of nucleic acid fragment sequences has a plurality of CpG sites, the first nucleic acid fragment sequence has a corresponding methylation pattern across the plurality of CpG sites, the methylation state of the first nucleic acid fragment sequence is a p-value, and the method further comprises determining the p-value of the first nucleic acid fragment sequence, at least in part, by comparison of the corresponding methylation pattern of the first nucleic acid fragment sequence to a corresponding distribution of methylation patterns of those nucleic acid fragment sequences in a healthy noncancer cohort dataset that each have the respective plurality of CpG sites.

P-value determination is further described in Example 5 in International Patent Application No. PCT/US2020/034317, entitled “Systems and Methods for Determining Whether a Subject has a Cancer Condition Using Transfer Learning,” filed May 22, 2020, and in U.S. patent application Ser. No. 16/352,602, entitled “Anomalous fragment detection and classification,” filed Mar. 13, 2019, now published as US2019/0287652, each of which is hereby incorporated herein by reference in its entirety. The goal of p-value determination can be to measure anomalous methylation in nucleic acid fragment sequences based on their corresponding methylation state vectors. For example, for each nucleic acid fragment in a biological sample, a determination is made as to whether the fragment is anomalously methylated (e.g., via analysis of sequence reads derived therefrom), relative to an expected methylation state vector using the methylation state vector corresponding to the fragment (e.g., where the expected methylation state vector is determined from sequence analysis of a cohort (plurality) of healthy subjects). The generation of methylation state vectors for such nucleic acid fragments (e.g., cell-free nucleic acid fragments) is disclosed above and, for example, in United States Patent Application Publication No. 2019/0287652, which is hereby incorporated herein by reference in its entirety.

In some embodiments, the healthy cohort comprises at least twenty subjects and the plurality of nucleic acid fragment sequences comprises at least 10,000 different corresponding methylation patterns. In some embodiments, the healthy cohort comprises at least 10, at least 20, at least 30, at least 40, at least 50, at least 60, at least 70, at least 80, at least 90, or at least 100 subjects. In some embodiments, the healthy cohort comprises between 1 and 10, between 10 and 50, between 50 and 100, between 100 and 500, between 500 and 1000, or more than 1000 subjects. In some embodiments, the plurality of nucleic acid fragment sequences comprises between 1 and 1000, between 1000 and 2000, between 2000 and 4000, between 4000 and 6000, between 6000 and 8000, between 8000 and 10,000, between 10,000 and 20,000, between 20,000 and 50,000, or more than 50,000 different corresponding methylation patterns.

In some embodiments, anomalous fragments are identified as fragments with over a threshold number of CpG sites and either with over a threshold percentage of the CpG sites methylated (hypermethylated) or with over a threshold percentage of CpG sites unmethylated (hypomethylated). In some embodiments, the threshold percentage of methylated and/or unmethylated CpG sites is at least 50%, at least 60%, at least 70%, at least 80%, at least 85%, at least 90%, or at least 95%. In some embodiments, the threshold percentage of methylated and/or unmethylated CpG sites is between 50% and 100%.

In some embodiments, a Markov model (e.g., a Hidden Markov Model “HMM”) is used to determine the probability that a sequence of methylation states (comprising, e.g., “M” for methylated and/or “U” for unmethylated) can be observed for each respective nucleic acid fragment sequence, given a set of probabilities that determine, for each state in the methylation pattern of the respective nucleic acid fragment sequence, the likelihood of observing the next state in the sequence. In some embodiments, the set of probabilities are obtained by training the HMM. In some embodiments, such training involves computing statistics (e.g., the probability that a first state will transition to a second state (the transition probability) and/or the probability that a given methylation state will be observed for a respective CpG site (the emission probability)), given an initial training dataset of observed methylation state sequences (e.g., methylation patterns) obtained from a cohort of non-cancer subjects. In some embodiments, the HMM is trained using supervised training (e.g., using samples where the underlying sequence as well as the observed states are known). In some alternative embodiments, the HMM is trained using unsupervised training (e.g., Viterbi learning, maximum likelihood estimation, expectation-maximization training, and/or Baum-Welch training). For example, an expectation-maximization algorithm such as the Baum-Welch algorithm estimates the transition and emission probabilities from observed sample sequences and generates a parameterized probabilistic model that best explains the observed sequences. Such algorithms iterate the computation of a likelihood function until the expected number of correctly predicted states is maximized.

In some embodiments, the p-value of the respective nucleic acid fragment sequence is determined by a method other than a Markov model or a Hidden Markov Model. In some embodiments, the p-value of the respective nucleic acid fragment sequence is determined using a mixture model. For example, a mixture model can detect an anomalous methylation pattern in a nucleic acid fragment sequence by determining the likelihood of a methylation state vector (e.g., a methylation pattern) for the respective nucleic acid methylation fragment based on the number of possible methylation state vectors of the same length and at the same corresponding genomic location. This can be executed by generating a plurality of possible methylation states for vectors of a specified length at each genomic position in a reference sequence (e.g., a human reference genome). Using the plurality of possible methylation states, the number of total possible methylation states and subsequently the probability of each predicted methylation state at the genomic position can be determined. The likelihood of a sample nucleic acid methylation fragment corresponding to a genomic position within the reference sequence can then be determined by matching the sample nucleic acid fragment sequence to a predicted (e.g., possible) methylation state and retrieving the calculated probability of the predicted methylation state. An anomalous methylation score is then calculated based on the probability of the sample nucleic acid fragment sequence.

In some embodiments, the p-value of the respective nucleic acid methylation fragment is determined using a learned representation. Any other suitable method of determining p-values is contemplated, as will be apparent to one skilled in the art.

In some embodiments, p-values (e.g., determined by any of the methods disclosed herein) are used as a filter to remove nucleic acid fragment sequences that are not sufficiently anomalous to be used as inputs (e.g., for a model) in the systems and methods for identifying variant alleles disclosed herein.

In some such embodiments, those nucleic acid fragment sequences that have a p-value below the threshold value are retained for further use in the method (e.g., as inputs to a model for identifying variant alleles as somatic or germline). For example, in some embodiments, the plurality of nucleic acid fragment sequences is filtered by removing each respective nucleic acid fragment sequence whose corresponding methylation pattern (e.g., methylation state vector) across a corresponding plurality of CpG sites in the respective fragment has a p-value that fails to satisfy a p-value threshold.

In some embodiments, the p-value threshold is between 0.001 and 0.20. In some embodiments, the threshold value is 0.01 (e.g., p can be <0.01 in such embodiments). In some embodiments, the threshold value is 0.001, 0,005, 0.01, 0.015, 0.02, 0.05, or 0.10. In some embodiments, the threshold value is between 0.0001 and 0.20. In some embodiments, the p-value threshold is satisfied for a methylation pattern from the subject when the corresponding methylation pattern for each respective cell-free fragment in the plurality of cell-free fragments has a p-value of 0.10 or less, 0.05 or less, or 0.01 or less.

Referring again to Block 210, in some embodiments, each indication in the (i) one or more indications of methylation state across the methylation state of each nucleic acid fragment sequence in the variant subset is a measure of central tendency of a methylation state p-value across the variant subset, a minimum methylation state p-value across the variant subset, a maximum methylation state p-value across the variant subset, or a measure of spread of a methylation state p-value across the variant subset.

For instance, in some embodiments, an indication in the one or more indications of methylation state across the variant subset is the measure of central tendency of a methylation state p-value across the variant subset, and the measure of central tendency is an arithmetic mean, a weighted mean, a midrange, a midhinge, a trimean, a Winsorized mean, a mean, or a mode of the methylation state p-value across the variant subset. In some embodiments, an indication in the one or more indications of methylation state across the variant subset is a measure of spread of a methylation state p-value across the variant subset, and the measure of spread is a standard deviation, a variance, a range, or an interquartile range of the methylation state p-value across the variant subset.

In some embodiments, the one or more indications of methylation state across the variant subset is a plurality of indications of methylation state across the variant subset comprising at least two, at least three, or all four of a measure of central tendency of a methylation state p-value across the variant subset, a minimum methylation state p-value across the variant subset, a maximum methylation state p-value across the variant subset, and a measure of spread of a methylation state p-value across the variant subset.

In some embodiments, the one or more indications of methylation state across the variant subset is a plurality of indications of methylation state across the variant subset comprising a mean p-value, a median p-value, a minimum p-value, a maximum p-value, and a standard deviation of p-values across the variant subset.

In some embodiments, the one or more indications of methylation state across the variant subset comprises a set of best ranked (e.g., most significant) p-values from the variant subset. For example, in some embodiments, the one or more indications of methylation across the variant subset comprises at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 of the best ranked (e.g., most significant) p-values from the variant subset. In some embodiments, the one or more indications of methylation across the variant subset comprises the top 50%, 40%, 30%, 20%, 10%, 9%, 8%, 7%, 6%, 5%, 4%, 3%, 2%, or the top 1% of the best ranked (e.g., most significant) p-values from the variant subset.

In some embodiments, the one or more indications of methylation state across the methylation state of each nucleic acid fragment in the variant subset comprises a methylation state vector and/or one or more distribution statistics thereof (e.g., a measure of central tendency across the variant subset, a minimum across the variant subset, a maximum across the variant subset, and a measure of spread across the variant subset).

In some embodiments, the one or more indications of methylation state across the methylation state of each nucleic acid fragment in the variant subset comprises a Beta-value and/or one or more distribution statistics thereof (e.g., a measure of central tendency across the variant subset, a minimum across the variant subset, a maximum across the variant subset, and a measure of spread across the variant subset).

In some embodiments, the one or more indications of methylation state across the methylation state of each nucleic acid fragment in the variant subset comprises an M-value and/or one or more distribution statistics thereof (e.g., a measure of central tendency across the variant subset, a minimum across the variant subset, a maximum across the variant subset, and a measure of spread across the variant subset).

In some embodiments, the one or more indications of methylation state across the methylation state of each nucleic acid fragment in the variant subset comprises an anomalous methylation score and/or one or more distribution statistics thereof (e.g., a measure of central tendency across the variant subset, a minimum across the variant subset, a maximum across the variant subset, and a measure of spread across the variant subset).

In some embodiments, the one or more indications of methylation state across the methylation state of each nucleic acid fragment in the variant subset comprises a mutual information score and/or one or more distribution statistics thereof (e.g., a measure of central tendency across the variant subset, a minimum across the variant subset, a maximum across the variant subset, and a measure of spread across the variant subset). Further details regarding mutual information scores are disclosed in U.S. Provisional Patent Application No. 62/948,129, titled “Cancer Classification using Patch Convolutional Neural Networks,” filed Dec. 13, 2019, which is hereby incorporated herein by reference in its entirety.

In some embodiments, the measure of central tendency is an arithmetic mean, a weighted mean, a midrange, a midhinge, a trimean, a Winsorized mean, a mean, or a mode of the methylation state p-value across the variant subset. In some embodiments, the measure of spread is a standard deviation, a variance, a range, or an interquartile range of the methylation state p-value across the variant subset.

In some embodiments, the one or more indications of methylation state across the methylation state of each nucleic acid fragment in the variant subset comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 200, at least 500, at least 800, or at least 1000 indications of methylation state across the variant subset. In some embodiments, the one or more indications of methylation state across the methylation state of each nucleic acid fragment in the variant subset comprises no more than 2000, no more than 1000, no more than 500, no more than 200, no more than 100, no more than 90, no more than 80, no more than 70, no more than 60, no more than 50, no more than 40, no more than 30, no more than 20, or no more than 10 indications of methylation state across the variant subset. In some embodiments, the one or more indications of methylation state across the methylation state of each nucleic acid fragment in the variant subset comprises from 3 to 10, from 5 to 20, from 10 to 50, from 20 to 100, from 50 to 200, from 100 to 500, from 300 to 1000, or from 500 to 2000 indications of methylation state across the variant subset. In some embodiments, the one or more indications of methylation state in the variant subset falls within another range starting no lower than 3 indications and ending no higher than 2000 indications of methylation state across the variant subset.

Referring to Block 214, in some embodiments, the method further comprises applying, to the trained binary classifier, (iii) one or more CpG site indications across the variant subset.

In some embodiments, a CpG site indication is a CpG count. For instance, in some embodiments, CpG counts are obtained by tallying the number of CpG sites in a nucleic acid fragment, based on the nucleic acid fragment sequence. In some embodiments, each nucleic acid fragment sequence in the variant subset has the same CpG count. In some embodiments, two or more nucleic acid fragment sequences in the variant subset have different CpG counts. In some embodiments, each nucleic acid fragment sequence in the variant subset has at least a minimum number of CpG sites (e.g., where the respective plurality of nucleic acid fragment sequences for the genomic position is filtered using a minimum or maximum CpG count).

In some embodiments, the minimum number of CpG sites is at least 1, 2, 3, 4, 5, 6, 7, 8, 9 or 10 CpG sites. In some embodiments, the minimum number of CpG sites is between 1 and 10, between 10 and 20, between 20 and 30, between 30 and 40, between 40 and 50, or more than 50 CpG sites.

In some embodiments, an indication in the one or more CpG site indications across the variant subset comprises a measure of central tendency of a CpG count across the variant subset, a minimum CpG count across the variant subset, a maximum CpG count across the variant subset, and a measure of spread of CpG count across the variant subset.

For instance, in some embodiments, an indication in the one or more CpG site indications across the variant subset is the measure of central tendency of a CpG count across the variant subset, and the measure of central tendency is an arithmetic mean, a weighted mean, a midrange, a midhinge, a trimean, a Winsorized mean, a mean, or a mode of the CpG count across the variant subset. In some embodiments, an indication in the one or more CpG site indications across the variant subset is a measure of spread of a CpG count across the variant subset, and the measure of spread is a standard deviation, a variance, a range, or an interquartile range of the CpG count across the variant subset.

In some embodiments, the one or more CpG indications across the variant subset is a plurality of CpG site indications across the variant subset comprising at least two, at least three, or all four of a measure of central tendency of a CpG count across the variant subset, a minimum CpG count across the variant subset, a maximum CpG count across the variant subset, and a measure of spread of CpG count across the variant subset.

In some embodiments, the one or more CpG indications across the variant subset is a plurality of CpG site indications across the variant subset comprising a CpG count, a median CpG count, a minimum CpG count, a maximum CpG count, and a standard deviation of CpG counts across the variant subset.

In some embodiments, the one or more CpG indications across the variant subset includes a genomic position of a CpG site and/or one or more distribution statistics thereof. In some embodiments, the one or more CpG indications across the variant subset includes a CpG density and/or one or more distribution statistics thereof. In some embodiments, the one or more CpG indications across the variant subset includes a genomic distance between two or more CpG sites and/or one or more distribution statistics thereof (e.g., a measure of central tendency across the variant subset, a minimum across the variant subset, a maximum across the variant subset, and a measure of spread across the variant subset).

In some embodiments, the one or more CpG indications across the variant subset comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, or at least 100 CpG indications across the variant subset. In some embodiments, the one or more CpG indications across the variant subset comprises no more than 200, no more than 100, no more than 90, no more than 80, no more than 70, no more than 60, no more than 50, no more than 40, no more than 30, no more than 20, or no more than 10 CpG indications across the variant subset. In some embodiments, the one or more CpG indications across the variant subset comprises from 3 to 10, from 5 to 20, from 10 to 50, from 20 to 100, or from 50 to 200 CpG indications across the variant subset. In some embodiments, the one or more CpG indications in the variant subset falls within another range starting no lower than 3 CpG indications and ending no higher than 200 CpG indications across the variant subset.

Referring to Block 216, in some embodiments, the applying, to the trained binary classifier, further applies one or more indications of methylation state across the reference subset.

In some embodiments, the one or more indications of methylation state across the reference subset is a p-value. In some embodiments, p-values for the reference subset are obtained using any of the methods disclosed herein, or any suitable substitutions, modifications, additions, deletions, and/or combinations thereof.

In some embodiments, each indication in the one or more indications of methylation state across the reference subset is a measure of central tendency of a methylation state p-value across the reference subset, a minimum methylation state p-value across the reference subset, a maximum methylation state p-value across the variant reference, or a measure of spread of a methylation state p-value across the reference subset.

For instance, in some embodiments, an indication in the one or more indications of methylation state across the reference subset is the measure of central tendency of a methylation state p-value across the reference subset, and the measure of central tendency is an arithmetic mean, a weighted mean, a midrange, a midhinge, a trimean, a Winsorized mean, a mean, or a mode of the methylation state p-value across the reference subset. In some embodiments, an indication in the one or more indications of methylation state across the reference subset is a measure of spread of a methylation state p-value across the reference subset, and the measure of spread is a standard deviation, a variance, a range, or an interquartile range of the methylation state p-value across the reference subset.

In some embodiments, the applying, to the trained binary classifier, further applies a plurality of indications of methylation state across the reference subset comprising at least two, at least three, or all four of a measure of central tendency of a methylation state p-value across the reference subset, a minimum methylation state p-value across the reference subset, a maximum methylation state p-value across the reference subset, and a measure of spread of a methylation state p-value across the reference subset.

In some embodiments, the one or more indications of methylation state across the reference subset is a plurality of indications of methylation state across the reference subset comprising a mean p-value, a median p-value, a minimum p-value, a maximum p-value, and a standard deviation of p-values across the reference subset.

In some embodiments, the one or more indications of methylation state across the reference subset comprises a set of best ranked (e.g., most significant) p-values from the reference subset. For example, in some embodiments, the one or more indications of methylation across the reference subset comprises at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 of the best ranked (e.g., most significant) p-values from the reference subset. In some embodiments, the one or more indications of methylation across the reference subset comprises the top 50%, 40%, 30%, 20%, 10%, 9%, 8%, 7%, 6%, 5%, 4%, 3%, 2%, or the top 1% of the best ranked (e.g., most significant) p-values from the reference subset.

In some embodiments, the one or more indications of methylation state across the methylation state of each nucleic acid fragment in the reference subset comprises a methylation state vector and/or one or more distribution statistics thereof (e.g., a measure of central tendency across the reference subset, a minimum across the reference subset, a maximum across the reference subset, and a measure of spread across the reference subset).

In some embodiments, the one or more indications of methylation state across the methylation state of each nucleic acid fragment in the reference subset comprises a Beta-value and/or one or more distribution statistics thereof (e.g., a measure of central tendency across the reference subset, a minimum across the reference subset, a maximum across the reference subset, and a measure of spread across the reference subset).

In some embodiments, the one or more indications of methylation state across the methylation state of each nucleic acid fragment in the reference subset comprises an M-value and/or one or more distribution statistics thereof (e.g., a measure of central tendency across the reference subset, a minimum across the reference subset, a maximum across the reference subset, and a measure of spread across the reference subset).

In some embodiments, the one or more indications of methylation state across the methylation state of each nucleic acid fragment in the reference subset comprises an anomalous methylation score and/or one or more distribution statistics thereof (e.g., a measure of central tendency across the reference subset, a minimum across the reference subset, a maximum across the reference subset, and a measure of spread across the reference subset).

In some embodiments, the one or more indications of methylation state across the methylation state of each nucleic acid fragment in the reference subset comprises a mutual information score and/or one or more distribution statistics thereof (e.g., a measure of central tendency across the reference subset, a minimum across the reference subset, a maximum across the reference subset, and a measure of spread across the reference subset). Further details regarding mutual information scores are disclosed in U.S. Provisional Patent Application No. 62/948,129, titled “Cancer Classification using Patch Convolutional Neural Networks,” filed Dec. 13, 2019, which is hereby incorporated herein by reference in its entirety.

In some embodiments, the one or more indications of methylation state across the methylation state of each nucleic acid fragment in the reference subset comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 200, at least 500, at least 800, or at least 1000 indications of methylation state across the reference subset. In some embodiments, the one or more indications of methylation state across the methylation state of each nucleic acid fragment in the reference subset comprises no more than 2000, no more than 1000, no more than 500, no more than 200, no more than 100, no more than 90, no more than 80, no more than 70, no more than 60, no more than 50, no more than 40, no more than 30, no more than 20, or no more than 10 indications of methylation state across the reference subset. In some embodiments, the one or more indications of methylation state across the methylation state of each nucleic acid fragment in the reference subset comprises from 3 to 10, from 5 to 20, from 10 to 50, from 20 to 100, from 50 to 200, from 100 to 500, from 300 to 1000, or from 500 to 2000 indications of methylation state across the reference subset. In some embodiments, the one or more indications of methylation state in the reference subset falls within another range starting no lower than 3 indications and ending no higher than 2000 indications of methylation state across the reference subset.

Referring to Block 218, in some embodiments, the applying, to the trained binary classifier, further applies one or more CpG site indications across the reference subset. In some embodiments, a CpG site indication is a CpG count (e.g., as described above).

In some embodiments, each nucleic acid fragment sequence in the reference subset has the same CpG count. In some embodiments, two or more nucleic acid fragment sequences in the reference subset have different CpG counts. In some embodiments, each nucleic acid fragment sequence in the reference subset has at least a minimum number of CpG sites (e.g., where the respective plurality of nucleic acid fragment sequences for the genomic position is filtered using a minimum or maximum CpG count). In some embodiments, the minimum number of CpG sites is at least 1, 2, 3, 4, 5, 6, 7, 8, 9 or 10 CpG sites. In some embodiments, the minimum number of CpG sites is between 1 and 10, between 10 and 20, between 20 and 30, between 30 and 40, between 40 and 50, or more than 50 CpG sites.

In some embodiments, an indication in the one or more CpG site indications across the reference subset comprises a measure of central tendency of a CpG count across the reference subset, a minimum CpG count across the reference subset, a maximum CpG count across the reference subset, and a measure of spread of CpG count across the reference subset.

For instance, in some embodiments, an indication in the one or more CpG site indications across the reference subset is the measure of central tendency of a CpG count across the reference subset, and the measure of central tendency is an arithmetic mean, a weighted mean, a midrange, a midhinge, a trimean, a Winsorized mean, a mean, or a mode of the CpG count across the reference subset. In some embodiments, an indication in the one or more CpG site indications across the reference subset is a measure of spread of a CpG count across the reference subset, and the measure of spread is a standard deviation, a variance, a range, or an interquartile range of the CpG count across the variant subset.

In some embodiments, the applying, to the trained binary classifier, further applies a plurality of CpG site indications across the reference subset, wherein the plurality of CpG site indications across the reference subset comprises at least two, at least three, or all four of a measure of central tendency of a CpG count across the reference subset, a minimum CpG count across the reference subset, a maximum CpG count across the reference subset, and a measure of spread of CpG count across the reference subset.

In some embodiments, the one or more CpG indications across the reference subset is a plurality of CpG site indications across the reference subset comprising a CpG count, a median CpG count, a minimum CpG count, a maximum CpG count, and a standard deviation of CpG counts across the reference subset.

In some embodiments, the one or more CpG indications across the reference subset comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, or at least 100 CpG indications across the reference subset. In some embodiments, the one or more CpG indications across the reference subset comprises no more than 200, no more than 100, no more than 90, no more than 80, no more than 70, no more than 60, no more than 50, no more than 40, no more than 30, no more than 20, or no more than 10 CpG indications across the reference subset. In some embodiments, the one or more CpG indications across the reference subset comprises from 3 to 10, from 5 to 20, from 10 to 50, from 20 to 100, or from 50 to 200 CpG indications across the reference subset. In some embodiments, the one or more CpG indications in the reference subset falls within another range starting no lower than 3 CpG indications and ending no higher than 200 CpG indications across the reference subset.

Referring again to Block 210, in some embodiments, the (ii) indication of a number of nucleic acid fragment sequences in the reference subset versus a number of nucleic acid fragment sequences in the variant subset comprises a count of nucleic acid fragment sequences in the reference subset. In some embodiments, the indication of a number of nucleic acid fragment sequences in the reference subset versus a number of nucleic acid fragment sequences in the variant subset comprises a count of nucleic acid fragment sequences in the variant subset. In some embodiments, the indication of a number of nucleic acid fragment sequences in the reference subset versus a number of nucleic acid fragment sequences in the variant subset comprises a ratio of the count of nucleic acid fragment sequences in the variant subset compared to the count of nucleic acid fragment sequences in the reference subset.

In some embodiments, the indications (e.g., the one or more indications of methylation state for the variant subset, one or more indications of methylation state for the reference subset, indication of a number of nucleic acid fragment sequences in the reference subset versus in the variant subset, the one or more CpG indications for the variant subset, and/or the one or more CpG indications for the reference subset) for application to the trained binary classifier are pooled (e.g., the variant subset and the reference subset) and binned into an input vector for the genomic position. In some embodiments, the pooled indications in the input vector are labeled as variant and/or reference.

In some embodiments, the indications for application to the trained binary classifier are faceted such that indications corresponding to the variant subset are binned into a first input vector for the variant subset for the genomic position and indications corresponding to the reference subset are binned into a second input vector for the reference subset for the genomic position.

In some instances, the indications in an input vector are applied as features to the trained binary classifier.

In some embodiments, the input vector has fixed length. In some embodiments, the input vector has variable length. In some embodiments, each genomic position in a plurality of genomic positions has an input vector of the same length or different lengths.

In some embodiments, an input vector for a respective genomic position comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 200, at least 500, at least 800, at least 1000, at least 2000, or at least 5000 indications (e.g., features). In some embodiments, an input vector for a respective genomic position comprises no more than 10,000, no more than 5000, no more than 2000, no more than 1000, no more than 500, no more than 200, no more than 100, no more than 90, no more than 80, no more than 70, no more than 60, no more than 50, no more than 40, no more than 30, no more than 20, or no more than 10 indications (e.g., features). In some embodiments, an input vector for a respective genomic position comprises from 3 to 10, from 5 to 20, from 10 to 50, from 20 to 100, from 50 to 200, from 100 to 500, from 300 to 1000, from 500 to 2000, or from 1000 to 10,000 indications. In some embodiments, an input vector for a respective genomic position comprises a plurality of indications falling within another range starting no lower than 3 indications and ending no higher than 10,000 indications (e.g., features).

Thus, in an example implementation, the identifying a variant allele at a respective genomic position in a subject as somatic or germline comprises providing a trained binary classifier with one or more input vectors, where the genomic position is for a candidate variant allele in the subject (e.g., identified as described above, with reference to Block 204) and the one or more input vectors includes a plurality of features (e.g., indications) for the respective genomic position. The plurality of features can include, for example, (i) one or more p-values and/or distribution statistics thereof, (ii) an indication of a number of variant versus reference nucleic acid fragment sequences, and (iii) one or more CpG counts and/or distribution statistics thereof, obtained for a plurality of nucleic acid fragment sequences that map to the genomic position. The trained classifier can then provide, as output, a determination of whether the variant is somatic or germline, based on the plurality of indications in the input vector.

Classifiers.

In some embodiments, the trained classifier is a trained logistic regression classifier or a multilayer perceptron classifier.

In some embodiments, the trained classifier is a trained decision tree classifier, a trained random forest classifier, a trained support vector machine classifier, a trained k-Nearest neighbors classifier, a trained nearest centroid classifier, a trained neural network classifier, or a trained naïve Bayes classifier. In some embodiments, the trained classifier is any of the classifiers disclosed below in Example 3.

In some embodiments, the trained classifier comprises a corresponding plurality of parameters (e.g., weights; see, for example, Definitions: Parameter).

In some embodiments, the trained classifier comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 parameters. In some embodiments, the trained classifier comprises at least 100, at least 500, at least 800, at least 1000, at least 2000, at least 3000, at least 4000, at least 5000, at least 6000, at least 7000, at least 8000, at least 9000, at least 10,000, at least 15,000, at least 20,000, or at least 30,000 parameters. In some embodiments, the trained classifier comprises no more than 30,000, no more than 20,000, no more than 15,000, no more than 10,000, no more than 9000, no more than 8000, no more than 7000, no more than 6000, no more than 5000, no more than 4000, no more than 3000, no more than 2000, no more than 1000, no more than 900, no more than 800, no more than 700, no more than 600, no more than 500, no more than 400, no more than 300, no more than 200, no more than 100, or no more than 50 parameters. In some embodiments, the trained classifier comprises from 2 to 20, from 2 to 200, from 2 to 1000, from 10 to 50, from 10 to 200, from 20 to 500, from 100 to 800, from 50 to 1000, from 500 to 2000, from 1000 to 5000, from 5000 to 10,000, from 10,000 to 15,000, from 15,000 to 20,000, or from 20,000 to 30,000 parameters. In some embodiments, the trained classifier comprises a plurality of parameters that falls within another range starting no lower than 2 parameters and ending no higher than 30,000 parameters.

In some embodiments, the trained classifier is a neural network comprising a plurality of hidden layers and a plurality of hidden neurons. For instance, in some embodiments, the trained classifier is a neural network, and the plurality of hidden layers comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 hidden layers. In some embodiments, the plurality of hidden layers comprises no more than 100, no more than 90, no more than 80, no more than 70, no more than 60, no more than 50, no more than 40, no more than 30, no more than 20, no more than 10, no more than 9, no more than 8, no more than 7, no more than 6, or no more than 5 hidden layers. In some embodiments, the plurality of hidden layers comprises from 1 to 5, from 1 to 10, from 1 to 20, from 10 to 50, from 2 to 80, from 5 to 100, from 10 to 100, from 50 to 100, or from 3 to 30 hidden layers. In some embodiments, the plurality of hidden layers falls within another range starting no lower than 1 layer and ending no higher than 100 layers.

In some embodiments, the trained classifier is a neural network, and each hidden neuron in the plurality of hidden neurons is associated with a respective one or more corresponding parameters (e.g., weights) in the corresponding plurality of parameters for the trained classifier. For instance, in some embodiments, the plurality of hidden neurons comprises from 2 to 20, from 2 to 200, from 2 to 1000, from 10 to 50, from 10 to 200, from 20 to 500, from 100 to 800, from 50 to 1000, from 500 to 2000, from 1000 to 5000, from 5000 to 10,000, from 10,000 to 15,000, from 15,000 to 20,000, or from 20,000 to 30,000 parameters. In some embodiments, the plurality of hidden neurons comprises at least as many hidden neurons as parameters in the corresponding plurality of parameters for the classifier.

In some embodiments, the trained classifier is a neural network, and each hidden neuron in the plurality of hidden neurons is associated with a first activation function type and/or a second activation function type.

In some embodiments, the first and/or the second activation function (e.g., for a respective hidden neuron) is selected from the group consisting of all or a combination of tanh, sigmoid, softmax, logistic, Gaussian, Boltzmann-weighted averaging, absolute value, linear, rectified linear unit (ReLU), leaky ReLU, exponential linear unit (eLU), bounded rectified linear, soft rectified linear, parameterized rectified linear, average, max, min, sign, square, square root, multiquadric, inverse quadratic, inverse multiquadric, polyharmonic spline, and thin-plate spline.

In some embodiments, the present disclosure provides a method of training a classifier (e.g., an untrained or partially untrained model) to identify a variant allele at a genomic position in a test subject as somatic or germline.

Classifier training can be performed by obtaining an identification of a reference allele at the genomic position. For each respective subject in a plurality of subjects, for each respective genomic position in a plurality of genomic positions, a procedure can be performed comprising obtaining an orthogonal call for the variant allele at the respective genomic position as one of somatic or germline for the respective subject and obtaining an identification of the variant allele at the respective genomic position for the respective subject. The method can further comprise obtaining a methylation state and a respective sequence of each nucleic acid fragment sequence in a respective plurality of nucleic acid fragment sequences in a sequencing dataset (e.g., comprising at least 1×10⁶ nucleic acid fragment sequences) derived from a biological sample obtained from the respective subject that map onto the respective genomic position.

The (a) identification of the reference allele at the respective genomic position and (b) respective sequence of each nucleic acid fragment sequence in the respective plurality of nucleic acid fragment sequences can be used to assign each nucleic acid fragment sequence in the respective plurality of nucleic acid fragment sequences that has the reference allele, at the respective genomic position, to a reference subset. Additionally, the (a) identification of the variant allele at the respective genomic position and (b) respective sequence of each nucleic acid fragment sequence in the respective plurality of nucleic acid fragment sequences can be used to assign each nucleic acid fragment sequence in the respective plurality of nucleic acid fragment sequences that has the variant allele, at the respective genomic position, to a variant subset.

The method can further include using, for each respective subject in the plurality of subjects, for each respective genomic position in the plurality of genomic positions, at least (i) one or more indications of methylation state across the methylation state of each nucleic acid fragment sequence in the variant subset for the respective subject for the respective genomic position, (ii) an indication of a number of nucleic acid fragment sequences in the reference subset versus a number of nucleic acid fragment sequences in the variant subset for the respective subject for the respective genomic position, and (iii) the orthogonal call for the variant allele at the respective genomic position as one of somatic or germline for the respective subject to train the classifier to identify a variant allele at a genomic position in a test subject as somatic or germline.

For instance, in some embodiments, the method can comprise applying the at least (i) one or more indications of methylation state, the (ii) indication of the number of nucleic acid fragment sequences in the reference subset versus the variant subset, and the (iii) orthogonal call for the variant allele as somatic or germline, to an untrained or partially untrained model, thus training the classifier to identify a variant allele at a genomic position in a test subject as somatic or germline.

In some embodiments, the untrained or partially untrained model comprises any of the classifiers disclosed herein (e.g., in the foregoing and/or in Example 3, below).

In some embodiments, the untrained or partially untrained model comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 parameters. In some embodiments, the untrained or partially untrained model comprises at least 100, at least 500, at least 800, at least 1000, at least 2000, at least 3000, at least 4000, at least 5000, at least 6000, at least 7000, at least 8000, at least 9000, at least 10,000, at least 15,000, at least 20,000, or at least 30,000 parameters. In some embodiments, the untrained or partially untrained model comprises no more than 30,000, no more than 20,000, no more than 15,000, no more than 10,000, no more than 9000, no more than 8000, no more than 7000, no more than 6000, no more than 5000, no more than 4000, no more than 3000, no more than 2000, no more than 1000, no more than 900, no more than 800, no more than 700, no more than 600, no more than 500, no more than 400, no more than 300, no more than 200, no more than 100, or no more than 50 parameters. In some embodiments, the untrained or partially untrained model comprises from 2 to 20, from 2 to 200, from 2 to 1000, from 10 to 50, from 10 to 200, from 20 to 500, from 100 to 800, from 50 to 1000, from 500 to 2000, from 1000 to 5000, from 5000 to 10,000, from 10,000 to 15,000, from 15,000 to 20,000, or from 20,000 to 30,000 parameters. In some embodiments, the untrained or partially untrained model comprises a plurality of parameters that falls within another range starting no lower than 2 parameters and ending no higher than 30,000 parameters.

In some embodiments, the plurality of training subjects comprise at least 20, at least 30, at least 40, at least 50, at least 60, at least 70, at least 80, at least 90, at least 100, at least 200, at least 300, at least 400, or at least 500 subjects. In some embodiments, the plurality of training subjects comprise at least 100, at least 500, at least 800, at least 1000, at least 2000, at least 3000, at least 4000, at least 5000, at least 6000, at least 7000, at least 8000, at least 9000, at least 10,000, or at least 20,000 subjects. In some embodiments, the plurality of training subjects comprise no more than 20,000, no more than 10,000, no more than 5000, no more than 4000, no more than 3000, no more than 2000, no more than 1000, no more than 900, no more than 800, no more than 700, no more than 600, no more than 500, no more than 400, no more than 300, or no more than 200 subjects. In some embodiments, the plurality of training subjects comprise between 20 and 500, between 100 and 800, between 50 and 1000, between 500 and 2000, between 1000 and 5000, or between 5000 and 10,000 subjects. In some embodiments, the plurality of training subjects fall within another range starting no lower than 20 subjects and ending no higher than 20,000 subjects.

In some embodiments, training the classifier comprises using a training dataset for the plurality of training subjects. In some embodiments, the training dataset comprises, in electronic form, a respective plurality of nucleic acid fragment sequences for each respective training subject in the plurality of training subjects. In some embodiments, the obtaining the plurality of nucleic acid fragment sequences, for each training subject in the plurality of training subjects, is performed using any of the methods disclosed herein, and/or any suitable substitutions, modifications, additions, deletions, and/or combinations thereof.

In some embodiments, the method comprises obtaining, for each respective training subject in the plurality of training subjects, a plurality of biological samples, where each respective biological sample in the plurality of biological samples for the respective subject is used to obtain a respective plurality of nucleic acid fragment sequences. For instance, in some embodiments, a first plurality of nucleic acid fragment sequences can be obtained from a first biological sample (e.g., cell-free nucleic acids from a liquid biological sample), and a second plurality of nucleic acid fragment sequences can be obtained from a second, matched biological sample from the same respective training subject (e.g., a healthy tissue sample or a solid tumor sample).

In some embodiments, the method comprises, for each respective training subject in the plurality of training subjects, sequencing a respective biological sample obtained from the respective training subject using a plurality of sequencing methods, each respective sequencing method generating a respective plurality of nucleic acid fragment sequences. For example, in some embodiments, a first plurality of nucleic acid fragment sequences can be obtained from a first sequencing method (e.g., WGS) of a respective biological sample obtained from the respective training subject, and a second plurality of nucleic acid fragment sequences can be obtained from a second sequencing method of the respective biological sample (e.g., WGBS and/or targeted methylation).

In some embodiments, any number of matched samples and/or matched sequencing assays can be performed for a respective training subject in the plurality of training subjects. For instance, in some embodiments, a first plurality of nucleic acid fragment sequences can be obtained using a first sequencing method of a first biological sample for a respective training subject (e.g., WGS on healthy tissue samples), and a second plurality of nucleic acid fragment sequences can be obtained using a second sequencing method, other than the first sequencing method, of a second biological sample different from the first biological sample, from the respective training subject (e.g., targeted methylation on cfDNA in a liquid biological sample).

In some embodiments, the classifier is trained using a training dataset obtained from the same biological sample type as the sequencing dataset for the test subject. For instance, in some embodiments, the classifier is trained using nucleic acid fragment sequences derived from solid tissue samples from a plurality of training subjects, and the method of identifying a variant as somatic or germline using the trained classifier is performed using nucleic acid fragment sequences derived from a solid tissue sample from a test subject. In some embodiments, the classifier is trained using a training dataset obtained from a different biological sample type as the sequencing dataset for the test subject. For instance, in some embodiments, the classifier is trained using nucleic acid fragment sequences derived from solid tissue samples from a plurality of training subjects, and the method of identifying a variant as somatic or germline using the trained classifier is performed using cell-free nucleic acid fragment sequences derived from a liquid biological sample from a test subject.

Alternately or additionally, in some embodiments, the classifier is trained using a training dataset obtained via the same sequencing method as used for the test subject. For instance, in some embodiments, the classifier is trained using nucleic acid fragment sequences obtained from whole genome sequencing (WGS) of tissue samples from a plurality of training subjects, and the identifying a variant as somatic or germline using the trained classifier is performed using nucleic acid fragment sequences obtained from whole genome sequencing (WGS) of a tissue sample from the test subject. In some embodiments, the classifier is trained using a training dataset obtained via a different sequencing method as used for the test subject. For instance, in some embodiments, the classifier is trained using nucleic acid fragment sequences obtained from whole genome sequencing (WGS) of tissue samples from a plurality of training subjects, and the identifying a variant as somatic or germline using the trained classifier is performed using nucleic acid fragment sequences obtained from targeted methylation of cell-free nucleic acids in a liquid biological sample from the test subject.

In some embodiments, the training dataset further comprises, for each respective training subject in the plurality of training subjects, a tumor fraction and/or a tumor mutational burden.

As defined above, tumor fraction can refer to the fraction of nucleic acid molecules in a sample that originates from a cancerous tissue of the subject compared to a noncancerous tissue (see, Definitions: “Tumor fraction”). Tumor fraction can be represented as a value from 0 to 1 or converted to a percentage (e.g., from 0 to 100). In some embodiments, the tumor fraction is between 10⁻⁶ and 0.999. In some embodiments, the tumor fraction is between 10⁻⁵ and 0.999. In some embodiments, the tumor fraction is between 10⁻⁴ and 0.999. In some embodiments, the tumor fraction is between 0.001 and 0.999. In some embodiments, the tumor fraction is between 0.01 and 0.99. In some embodiments, the tumor fraction is between 10⁻⁵ and 0.04, between 10⁴ and 0.02, between 0.001 and 0.5, or between 0.001 and 0.1. In some embodiments, the tumor fraction is no more than 0.3, no more than 0.2, no more than 0.1, no more than 0.09, no more than 0.08, no more than 0.07, no more than 0.06, no more than 0.05, no more than 0.04, no more than 0.03, no more than 0.02, no more than 0.01, no more than 0.009, no more than 0.008, no more than 0.007, no more than 0.006, no more than 0.005, no more than 0.004, no more than 0.003, no more than 0.002, no more than 0.001, no more than 10⁴, or no more than 10⁻⁵. In some embodiments, the tumor fraction is at least 10, at least 0.001, at least 0.005, at least 0.01, at least 0.05, at least 0.1, at least 0.2, at least 0.3, or at least 0.5. In some embodiments, the tumor fraction falls within another range starting no lower than 10⁻⁶ and ending no higher than 0.999.

As defined above, tumor mutation burden refers to a measure of the mutations in a cancer per unit of the patient's genome (see, Definitions: “Tumor mutational burden”). In some embodiments, the tumor mutational burden is measured in a number of mutations per megabase (Mb) (e.g., of the patient's genome and/or coding sequence). In some embodiments, the tumor mutational burden is between 0.0001 and 5, between 0.001 and 5, between 0.001 and 1, or between 0.1 and 5 mutations per Mb. In some embodiments, the tumor mutational burden is between 5 and 10 mutations per Mb. In some embodiments, the tumor mutational burden is between 10 and 20, between 10 and 30, between 10 and 50, or between 10 and 100 mutations per Mb. In some embodiments, the tumor mutation burden is no more than 50, no more than 30, no more than 20, no more than 10, no more than 9, no more than 8, no more than 7, no more than 6, no more than 5, no more than 4, no more than 3, no more than 2, no more than 1, no more than 0.5, no more than 0.1, no more than 0.05, no more than 0.01, no more than 0.005, no more than 0.001, no more than 0.0005, or no more than 0.0001 mutations per Mb. In some embodiments, the tumor mutation burden is at least 0.001, at least 0.005, at least 0.01, at least 0.05, at least 0.1, at least 0.5, at least 1, at least 5, or at least 10 mutations per Mb. In some embodiments, the tumor mutation burden falls within another range starting no lower than 0.0001 mutations per Mb and ending no higher than 100 mutations per Mb.

In some embodiments, the training dataset comprises a weighting factor and/or a dilution factor for one or more training subjects in the plurality of training subjects (e.g., to account for differences in sample type and/or tumor fraction).

In some embodiments, the training dataset is filtered (e.g., using any of the filters disclosed herein; see, for example, the above section entitled “Assigning subsets”). In some embodiments, the filtering comprises removing genomic positions from the plurality of genomic positions, across all training subjects in the plurality of training subjects.

In some embodiments, the filtering comprises removing training subjects from the plurality of training subjects. For instance, in some embodiments, if all of the genomic positions in the plurality of genomic positions for a respective training subject fail to satisfy a filtering criterion (e.g., all genomic positions for the training subject are removed from the dataset), then the corresponding plurality of nucleic acid fragment sequences for the respective training subject is removed from the dataset.

Any suitable sample type, tissue type, sample collection, sequencing method, processing and/or bioinformatics analysis may be used to obtain a training dataset for one or more training subjects as for a test subject, as disclosed herein, and/or any substitutions, modifications, additions, deletions, and/or combinations thereof.

In some embodiments, other aspects of training the classifier (e.g., for each respective subject in a plurality of subjects, for each respective genomic position in a plurality of genomic positions), including subjects, samples, obtaining identifications variant and reference alleles, sequencing (e.g., methylation sequencing), processing nucleic acid fragment sequences, obtaining methylation states, assigning reference and variant subsets, and obtaining features, etc., are performed using any of the methods disclosed herein with respect to systems and methods of identifying variant alleles as somatic or germline (e.g., including subjects, samples, obtaining identifications variant and reference alleles, sequencing (e.g., methylation sequencing), processing nucleic acid fragment sequences, obtaining methylation states, assigning reference and variant subsets, and obtaining features, etc.), and/or using any suitable substitutions, modifications, additions, deletions, and/or combinations thereof.

As described above, in some embodiments, the training the classifier comprises obtaining an orthogonal call for the variant allele at the respective genomic position as one of somatic or germline for each respective subject in a plurality of subjects, for each respective genomic position in a plurality of genomic positions. The training dataset thus includes, for each genomic position of a variant of interest, for each respective subject, a corresponding label that the variant is a somatic variant or a germline variant.

In some embodiments, the orthogonal call for the variant allele is determined using a comparison between an aberrant sample and a reference sample. For instance, as described below in Example 6, in some embodiments, an orthogonal call for a variant allele is determined using and analysis between patient-matched tumor samples and normal tissue references. The orthogonal call (e.g., somatic or germline label) is then used as an input, with the plurality of indications for each training subject, to train the classifier.

Generally, training a classifier (e.g., logistic regression model, a neural network, and/or another suitable model) comprises updating the plurality of parameters for the respective classifier through backpropagation (e.g., gradient descent). First, a forward propagation is performed, in which input data is accepted into the untrained or partially untrained model, and an output is calculated based on the selected activation function and an initial set of parameters (e.g., weights). A backward pass can then be performed by calculating an error gradient for each respective parameter, where the error for each parameter is determined by calculating a loss (e.g., error) based on the output (e.g., the predicted value) and the input data (e.g., the expected value or true labels).

Parameters can then be updated by adjusting the value based on the calculated loss metered by a predetermined learning rate hyperparameter that dictates the degree or severity to which parameters are updated (e.g., small adjustments versus large adjustments), thereby training the untrained or partially untrained model.

For example, in some general embodiments of machine learning, backpropagation is a method of training an untrained or partially untrained model comprising a plurality of parameters (e.g., embeddings). The output of an untrained or partially untrained model (e.g., the identification of a variant as somatic or germline) can be generated using a set of arbitrarily selected initial parameters. The output is then compared with the original input (e.g., the orthogonal call of the variant allele of the respective training subject at the respective genomic position) by evaluating an error function to compute an error (e.g., using a loss function). The parameters can then be updated such that the error is minimized (e.g., according to the loss function). In some embodiments, any one of a variety of backpropagation algorithms and/or methods are used to update the plurality of parameters.

In some embodiments, the error is computed using an error function (e.g., a loss function). In some embodiments, the loss function is mean square error, quadratic loss, mean absolute error, mean bias error, hinge, multi-class support vector machine, and/or cross-entropy. In some embodiments, training the untrained or partially untrained model comprises computing an error in accordance with a gradient descent algorithm and/or a minimization function.

In some embodiments, the error function is used to update one or more parameters in an untrained or partially untrained model by adjusting the value of the one or more parameters by an amount proportional to the calculated loss, thereby training the model. In some embodiments, the amount by which the parameters are adjusted is metered by a predetermined learning rate that dictates the degree or severity to which parameters are updated (e.g., smaller or larger adjustments). In some embodiments, the learning rate is a hyperparameter that can be selected by a practitioner.

In some embodiments, training the untrained or partially untrained model forms a trained classifier following a first evaluation of an error function. In some such embodiments, training the untrained or partially untrained model forms a trained classifier following a first updating of one or more parameters based on a first evaluation of an error function. In some alternative embodiments, training the untrained or partially untrained model forms a trained classifier following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function. In some such embodiments, training the untrained or partially untrained model forms a trained classifier following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million updatings of one or more parameters based on the at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function.

In some embodiments, training the untrained or partially untrained model forms a trained classifier when the model satisfies a minimum performance requirement. For example, in some embodiments, training the untrained or partially untrained model forms a trained classifier when the error calculated for the trained classifier, following an evaluation of an error function across one or more training datasets for a respective one or more training subjects, satisfies an error threshold. In some embodiments, the error calculated by the error function across one or more training datasets for a respective one or more training subjects satisfies an error threshold when the error is less than 20 percent, less than 18 percent, less than 15 percent, less than 10 percent, less than 5 percent, or less than 3 percent.

In some embodiments, the minimum performance requirement is satisfied based on a validation training. In some embodiments, validation training is performed through K-fold cross-validation.

In some embodiments, classifier training is performed on a plurality of machines (e.g., computers and/or systems). In some embodiments, using the classifier to variant allele at a genomic position in a test subject as somatic or germline is performed on a plurality of machines (e.g., computers and/or systems).

In some embodiments, classifier training further comprises fixing (e.g., freezing) one or more parameters in the plurality of parameters, thereby obtaining a corresponding trained classifier that can be used to perform determination and/or classification (e.g., of a variant allele at a genomic position as somatic or germline).

Any other model parameters and architectures suitable for training are contemplated, as will be apparent to one skilled in the art.

Applications.

Referring to Block 220, in some embodiments, when the variant allele at the genomic position is determined by the trained binary classifier to be germline, the method further comprises using the variant allele in the test subject to determine a cancer risk of the test subject. For example, in some embodiments, the genomic position is the BRCA1 or BRCA2 locus, the variant allele at the genomic position is determined by the trained binary classifier to be germline, and the method further comprises determining that the test subject is at risk for breast cancer.

Referring to Block 222, in some embodiments, when the variant allele at the genomic position is determined by the trained binary classifier to be germline, the method further comprises using the variant allele in the test subject to predict an ethnicity of the subject. For instance, germline variations in cancer genes have been reported to be ethnicity-specific, such that different variant alleles for a given locus are overrepresented in various ethnic populations. Thus, for a respective subject, a variant allele at a locus for a cancer gene (e.g., BRCA1 or BRCA2) can be used to determine ethnicity and assess cancer risk for the respective ethnicity.

In some embodiments, when the variant allele at the genomic position is determined by the trained binary classifier to be somatic, the method further comprises using the variant allele in the test subject to make a clinical determination of a disease. In some implementations, a clinical determination of a disease is a diagnosis, determining the stage of disease, monitoring progression, a prognosis, prescribing or administering a treatment, matching or recommending enrollment in a clinical trial, monitoring the development of additional complications or risks over time, and/or evaluating an efficacy of treatment. In some embodiments, the disease is cancer. In some embodiments, the disease is clonal hematopoiesis of indeterminate potential (CHIP), cardiovascular risk, nonalcoholic fatty liver disease (NAFLD), and/or nonalcoholic steatohepatitis (NASH).

For example, in some embodiments, the genomic position is the KRAS locus, the variant allele at the genomic position is determined by the trained binary classifier to be somatic, and the method further comprises using the variant allele to diagnose the patient with cancer (e.g., pancreatic, colorectal, and/or lung cancer).

In some embodiments, when the variant allele at the genomic position is determined by the trained binary classifier to be somatic, the method further comprises using the variant allele in the test subject to determine a tumor mutational burden of the subject (e.g., a normalized count of somatic variants per unit of base pairs). Typical methods for calculating tumor mutational burden generally make use of a tumor sample and a normal control sample (e.g., a normal reference). In some embodiments, the method provides a supplemental method (e.g., using a liquid biological sample) for using a variant allele in a test subject to determine the tumor mutational burden in the subject.

Referring to Block 224, in some embodiments, when the variant allele at the genomic position is determined by the trained binary classifier to be somatic, the method further comprises using the variant allele in the test subject to determine a tumor fraction of the subject. For instance, in some embodiments, if the biological sample for a respective test subject is derived from cell-free nucleic acids, the cell-free nucleic acids may exhibit an appreciable tumor fraction. In some embodiments, the corresponding tumor fraction in the respective test subject is at least two percent, at least five percent, at least ten percent, at least fifteen percent, at least twenty percent, at least twenty-five percent, at least fifty percent, at least seventy-five percent, at least ninety percent, at least ninety-five percent, or at least ninety-eight percent. In some embodiments, the corresponding tumor fraction in the respective test subject is no more than 60%, no more than 50%, no more than 40%, no more than 30%, no more than 20%, no more than 10%, no more than 5%, no more than 1%, or no more than 0.1%. In some such embodiments, such tumor fraction estimates are used to detect cancer in the subject, as described below in Example 3.

Tumor fraction and/or tumor mutational burden can be used, in some implementations, for additional diagnostic applications. For instance, tumor fraction and/or tumor mutational burden can be used to assess or monitor the effectiveness of cancer treatments (e.g., chemotherapy, immunotherapy, etc.).

In some embodiments, the method comprises obtaining a tumor fraction estimate of a test subject at a first time point and a second time point, where a diagnosis of the test subject is changed when the tumor fraction estimate of the subject is observed to change by a threshold amount between the first and the second time point. For instance, in some embodiments, the diagnosis is changed from having cancer to being in remission. As another example, in some embodiments, the diagnosis is changed from not having cancer to having cancer. As another example, in some embodiments, the diagnosis is changed from having a first stage of a cancer to having a second stage of a cancer. As another example, in some embodiments, the diagnosis is changed from having a second stage of a cancer to having a third stage of a cancer. As still another example, in some embodiments, the diagnosis is changed from having a third stage of a cancer to having a fourth stage of a cancer. As still another example, in some embodiments, the diagnosis is changed from having a cancer that has not metastasized to having a cancer that has metastasized.

In some embodiments, a prognosis of the test subject is changed when the tumor fraction estimate of the subject is observed to change by a threshold amount between the first and the second time point. For example, in some embodiments, the prognosis involves life expectancy and the prognosis is changed from a first life expectancy to a second life expectancy, where the first and second life expectancy differ in their duration. In some embodiments, the change in prognosis increases the life expectancy of the subject. In some embodiments, the change in prognosis decreases the life expectancy of the subject.

In some embodiments, a treatment of the test subject is changed when the tumor fraction estimate of the subject is observed to change by a threshold amount between the first and the second time point. In some embodiments, the changing of the treatment comprises initiating a cancer medication, increasing the dosage of a cancer medication, stopping a cancer medication, or decreasing the dosage of the cancer medication.

In some embodiments, a treatment regimen is applied to the test subject based, at least in part, on a value of the tumor fraction estimate and/or an identification of a variant at a genomic position as somatic or germline for the test subject. For instance, in some embodiments, the method further comprises, when the variant allele at the genomic position is determined by the trained binary classifier to be somatic, administering a first treatment to the test subject, and when the variant allele at the genomic position is determined by the trained binary classifier to be germline, administering a second treatment to the test subject.

In some embodiments, the treatment regimen comprises applying an agent for cancer to the test subject. In some embodiments, the agent for cancer is a hormone, an immune therapy, radiography, or a cancer drug. In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof.

In some embodiments, the test subject has been treated with an agent for cancer and the tumor fraction estimate and/or the identification of a variant at a genomic position as somatic or germline for the test subject is used to evaluate a response of the subject to the agent for cancer. Details of the agent for cancer are described elsewhere herein.

In some embodiments, the test subject has been treated with an agent for cancer and the tumor fraction estimate and/or the identification of a variant at a genomic position as somatic or germline for the test subject is used to determine whether to intensify or discontinue the agent for cancer in the test subject. For instance, in some embodiments, observation of at least a tumor fraction estimate (e.g., greater than 0.05, 0.10, 0.15, 0.20, 0.25, or 0.30, etc.) is used as a basis for intensifying (e.g., increasing the dosage, increasing radiation level in radiation treatment, etc.) of the agent for cancer in the test subject. In some embodiments, observation of less than a threshold tumor fraction estimate (e.g., less than 0.30, 0.25, 0.20, 0.15, 0.10, 0.05, or 0.01, etc.) is used as a basis for discontinuing use of the agent for cancer in the test subject.

In some embodiments, the test subject has been subjected to a surgical intervention to address the cancer and the tumor fraction estimate and/or the identification of a variant at a genomic position as somatic or germline for the test subject is used to evaluate a condition of the test subject in response to the surgical intervention. In some embodiments the condition is a metric based upon the tumor fraction estimate and/or the identification of a variant at a genomic position as somatic or germline using the methods provided in the present disclosure.

Methods for determining tumor fraction and tumor mutational burden are described in further detail in U.S. patent application Ser. No. 17/185,885, filed Feb. 25, 2021, entitled “Systems and Methods for Calling Variants using Methylation Sequencing Data,” and PCT Application No. PCT/US2021/019746, filed February 2021, entitled “Systems and Methods for Calling Variants using Methylation Sequencing Data,” each of which is hereby incorporated herein by reference in its entirety.

In some embodiments, the systems and methods of the present disclosure comprise using the identification of a variant at a genomic position as somatic or germline for the test subject to detect contamination. For instance, in some embodiments, the identification of a variant at a genomic position as somatic or germline for the test subject are used to detect cross-contamination using the techniques disclosed in U.S. patent application Ser. No. 15/900,645, entitled “Detecting cross-contamination in sequencing data using regression techniques,” filed Feb. 20, 2018 and published as US 2018/0237838, U.S. patent application Ser. No. 16/019,315, entitled “Detecting cross-contamination in sequencing data,” filed Jun. 26, 2018 and published as US 2018/0373832, and/or U.S. Application No. 63/080,670, entitled “Detecting cross-contamination in sequencing data,” filed Sep. 18, 2020.

### Additional Embodiments

Referring to Block 226, in some embodiments, the method further comprises repeating the method for each genomic position in a plurality of genomic positions, thereby identifying a plurality of variants for the test subject, and for each respective variant in the plurality of variants, whether the respective variant is somatic or germline.

In some embodiments, the plurality of variants comprises 200 variants.

In some embodiments, the plurality of variants comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 10,000, or at least 20,000 variants. In some embodiments, the plurality of variants comprises no more than 20,000, no more than 10,000, no more than 5000, no more than 4000, no more than 3000, no more than 2000, no more than 1000, no more than 900, no more than 800, no more than 700, no more than 600, no more than 500, no more than 400, no more than 300, no more than 200, no more than 100, no more than 90, no more than 80, no more than 70, no more than 60, no more than 50, or no more than 20 variants. In some embodiments, the plurality of variants is from 10 to 50, from 50 to 100, from 100 to 500, from 500 to 1000, from 1000 to 5000, from 5000 to 10,000, or from 10,000 to 20,000 variants. In some embodiments, the plurality of variants falls within another range starting no lower than 10 variants and ending no higher than 20,000 variants.

In some embodiments, each respective variant in the plurality of variants is a clinically actionable variant (e.g., a cancer gene). Suitable embodiments for clinically actionable variants can include any of the embodiments disclosed herein (see, for example, the section entitled “Reference and variant alleles,” above). In some embodiments, the plurality of variants is a panel of clinically actionable variants (e.g., cancer genes of interest).

In some embodiments, the plurality of variants is filtered. Suitable methods for filtering the plurality of variants include any of the embodiments for filtering variant calls, genomic positions, and/or nucleic acid fragment sequences as disclosed in detail here (see, for example, the foregoing sections entitled “Variant calling,” “Assigning subsets,” and “Input indications”), or any substitutions, modifications, additions, deletions, and/or combinations thereof, as will be apparent to one skilled in the art.

In some embodiments, the method further comprises removing a respective variant from the plurality of variants when the respective variant fails to satisfy a quality metric.

In some embodiments, the quality metric is a minimum variant allele fraction in the respective plurality of nucleic acid fragment sequences, in electronic form, that map to the genomic position of the respective variant call. In some embodiments, the minimum variant allele fraction is ten percent.

In some embodiments, the quality metric is a maximum variant allele fraction in the respective plurality of nucleic acid fragment sequences, in electronic form, that map to the genomic position of the respective variant. In some embodiments, the maximum variant allele fraction is ninety percent.

In some embodiments, the quality metric is a minimum depth in the respective plurality of nucleic acid fragment sequences that map to the genomic position of the respective variant. In some embodiments, the minimum depth is ten.

Additional embodiments for quality metrics that are contemplated for use in the present disclosure include quality metrics described in the foregoing section “Variant calling.”

Another aspect of the present disclosure provides a computing system, comprising one or more processors and memory storing one or more programs to be executed by the one or more processor, the one or more programs comprising instructions for performing any of the methods disclosed above alone or in combination.

Still another aspect of the present disclosure provides a non-transitory computer readable storage medium storing one or more programs configured for execution by a computer, where the one or more programs comprise instructions for performing any of the methods disclosed above alone or in combination.

### ADDITIONAL EXAMPLE EMBODIMENTS

### Example 1—Obtaining a Plurality of Sequence Reads

FIG. 7 is a flowchart of method 700 for preparing a nucleic acid sample for sequencing according to some embodiments of the present disclosure. The method 700 included, but was not limited to, the following steps. For example, any step of method 700 may comprise a quantitation sub-step for quality control or any other laboratory assay procedures.

Referring to Block 702, a nucleic acid sample (DNA or RNA) was extracted from a subject. The sample may be any subset of the human genome, including the whole genome. The sample may have been extracted from a subject known to have or suspected of having cancer. The sample may have include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may use surgery. The extracted sample may have comprised cfDNA and/or ctDNA. For healthy individuals, the human body may naturally clear out cfDNA and other cellular debris. If a subject has a cancer or disease, ctDNA in an extracted sample may have been present at a detectable level for diagnosis.

Referring to Block 704, a sequencing library was prepared. During library preparation, unique molecular identifiers (UMI) were added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs were degenerate base pairs that serve as a unique tag that can be used to identify sequence reads originating from a specific DNA fragment. During PCR amplification following adapter ligation, the UMIs were replicated along with the attached DNA fragment. This provided a way to identify sequence reads that came from the same original fragment in downstream analysis.

Referring to Block 706, targeted DNA sequences were enriched from the library. During enrichment, hybridization probes (also referred to herein as “probes”) were used to target, and pull down, nucleic acid fragments informative for the presence or absence of cancer (or disease), cancer status, or a cancer classification (e.g., cancer class or tissue of origin). For a given workflow, in some embodiments, the probes were designed to anneal (or hybridize) to a target (complementary) strand of DNA. In some embodiments each probe was between 8 and 5000 bases in length, between 12 and 2500 bases in length, or between 15 and 1225 bases in length. In some embodiments, the target strand have the “positive” strand (e.g., the strand transcribed into mRNA, and subsequently translated into a protein) or the complementary “negative” strand. In some embodiments the probes may have ranged in length from tens, hundreds or thousands of base pairs.

In some embodiments, the probes were designed based on a methylation site panel.

In some embodiments, the probes were designed based on a panel of targeted genes and/or genomic regions to analyze particular mutations or target regions of the genome (e.g., of the human or another organism) that are suspected to correspond to certain cancers or other types of diseases. For instance, in some embodiments, each of the probes uniquely mapped to a genomic region described in International Patent Publication Nos. WO2020154682A3, WO2020/069350A1, or WO2019/195268A2, each of which is hereby incorporated by reference.

In some embodiments, the probes covered overlapping portions of a target region. With reference to Block 708, in some embodiments the probes were used to generate sequence reads of the nucleic acid sample.

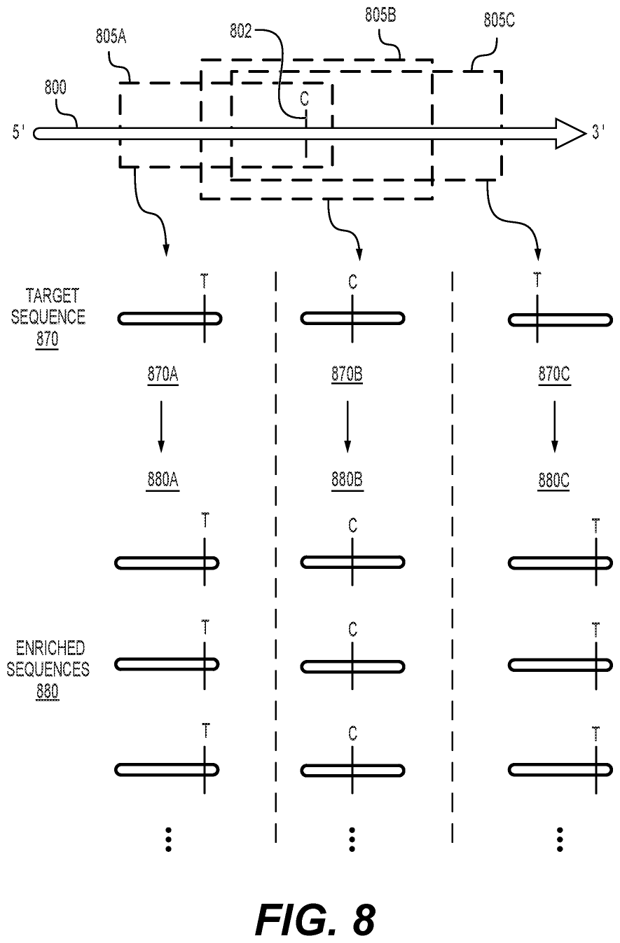

FIG. 8 is a graphical representation of the process for obtaining sequence reads according to one embodiment. FIG. 8 depicts one example of a nucleic acid segment 800 from the sample. Here, the nucleic acid segment 800 can be a single-stranded nucleic acid segment. In some embodiments, the nucleic acid segment 800 was a double-stranded cfDNA segment. The illustrated example depicts three regions 805A, 805B, and 805C of the nucleic acid segment that can be targeted by different probes. Specifically, each of the three regions 805A, 805B, and 805C includes an overlapping position on the nucleic acid segment 800. An example overlapping position is depicted in FIG. 8 as the cytosine (“C”) nucleotide base 802. The cytosine nucleotide base 802 is located near a first edge of region 805A, at the center of region 805B, and near a second edge of region 805C.

In some embodiments, one or more (or all) of the probes were designed based on a gene panel or methylation site panel to analyze particular mutations or target regions of the genome (e.g., of the human or another organism) that are suspected to correspond to certain cancers or other types of diseases. By using a targeted gene panel or methylation site panel rather than sequencing all expressed genes of a genome, also known as “whole-exome sequencing,” the method 800 may be used to increase sequencing depth of the target regions, where depth refers to the count of the number of times a given target sequence within the sample has been sequenced. Increasing sequencing depth reduces used input amounts of the nucleic acid sample. For instance, in some embodiments, a targeted gene panel or methylation site panel comprises a plurality of probes where each of the probes uniquely maps to a genomic region described in International Patent Publication Nos. WO2020154682A3, WO2020/069350A1, or WO2019/195268A2, each of which is hereby incorporated by reference.

Hybridization of the nucleic acid sample 800 using one or more probes results in an understanding of a target sequence 870. As shown in FIG. 8, the target sequence 870 is the nucleotide base sequence of the region 805 that is targeted by a hybridization probe. The target sequence 870 can also be referred to as a hybridized nucleic acid fragment. For example, target sequence 870A corresponds to region 805A targeted by a first hybridization probe, target sequence 870B corresponds to region 805B targeted by a second hybridization probe, and target sequence 870C corresponds to region 805C targeted by a third hybridization probe. Given that the cytosine nucleotide base 802 is located at different locations within each region 805A-C targeted by a hybridization probe, each target sequence 870 includes a nucleotide base that corresponds to the cytosine nucleotide base 802 at a particular location on the target sequence 870.

After a hybridization step, the hybridized nucleic acid fragments were captured and may also be amplified using PCR. For example, the target sequences 870 can be enriched to obtain enriched sequences 880 that can be subsequently sequenced. In some embodiments, each enriched sequence 880 was replicated from a target sequence 870. Enriched sequences 880A and 880C that were amplified from target sequences 870A and 870C, respectively, also include the thymine nucleotide base located near the edge of each sequence read 880A or 880C. As used hereafter, the mutated nucleotide base (e.g., thymine nucleotide base) in the enriched sequence 880 that was mutated in relation to the reference allele (e.g., cytosine nucleotide base 802) was considered as the alternative allele. Additionally, each enriched sequence 880B amplified from target sequence 870B included the cytosine nucleotide base located near or at the center of each enriched sequence 880B.

Referring again to Block 708 of FIG. 7, sequence reads were generated from the enriched DNA sequences, e.g., enriched sequences 880 shown in FIG. 8. Sequencing data may be acquired from the enriched DNA sequences. For example, the method 800 may include next-generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing was performed using sequencing-by-synthesis with reversible dye terminators.

In some embodiments, the sequence reads were aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In some embodiments, an average sequence read length of a corresponding plurality of sequence reads that was obtained by the methylation sequencing for a respective fragment was between 140 and 280 nucleotides.

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as methylation state determination.

### Example 2—Generation of a Methylation State Vector in Accordance with Some Embodiments of the Present Disclosure

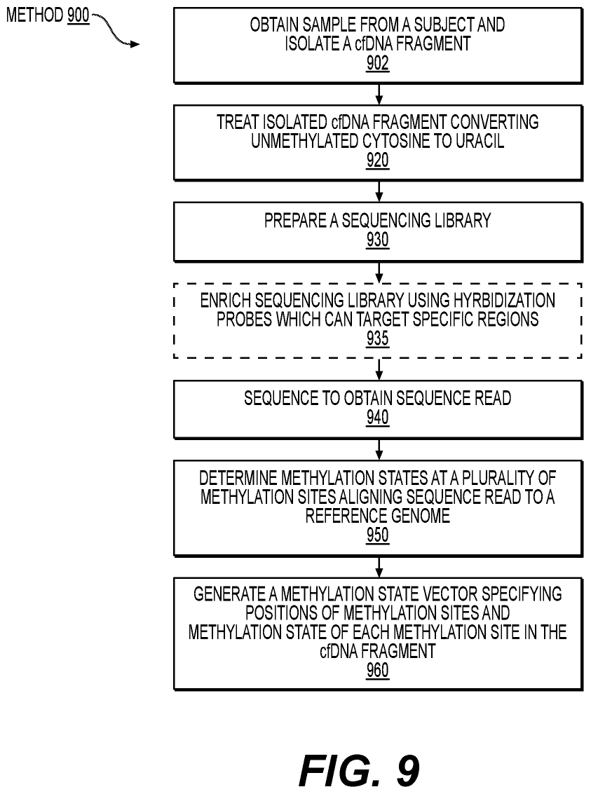

FIG. 9 is a flowchart describing a process 900 of sequencing a fragment of cfDNA to obtain a methylation state vector, according to an embodiment in accordance with the present disclosure.

Referring to Block 902, the cfDNA fragments were obtained from the biological sample. Referring to Block 920, the cfDNA fragments were treated to convert unmethylated cytosines to uracils. In some embodiments, the cfDNA was subjected to a bisulfite treatment that converts the unmethylated cytosines of the fragment of cfDNA to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) was used for the bisulfite conversion in some embodiments. In other embodiments, the conversion of unmethylated cytosines to uracils was accomplished using an enzymatic reaction. For example, the conversion can use a commercially available kit for converting unmethylated cytosines to uracils, such as APOBEC-Seq (NEBiolabs, Ipswich, Mass.).

From the converted cfDNA fragments, a sequencing library is prepared (Block 930). Optionally, the sequencing library is enriched (Block 935) for cfDNA fragments, or genomic regions, that are informative for cancer status using a plurality of hybridization probes. The hybridization probes are short oligonucleotides capable of hybridizing to particularly specified cfDNA fragments, or targeted regions, and enriching for those fragments or regions for subsequent sequencing and analysis. Hybridization probes may be used to perform targeted, high-depth analysis of a set of specified CpG sites of interest to the researcher. Once prepared, the sequencing library or a portion thereof can be sequenced to obtain a plurality of sequence reads (Block 940). The sequence reads may be in a computer-readable, digital format for processing and interpretation by computer software.

From the sequence reads, a location and methylation state for each of CpG site was determined based on the alignment of the sequence reads to a reference genome (Block 950). A methylation state vector for each fragment specifying a location of the fragment in the reference genome (e.g., as specified by the position of the first CpG site in each fragment, or another similar metric), a number of CpG sites in the fragment, and the methylation state of each CpG site in the fragment (Block 960).

### Example 3—Ability to Detect Cancer as a Function of cfDNA Fraction

In some embodiments, the method further comprises training a classifier to determine a cancer condition of the subject or a likelihood of the subject obtaining the cancer condition using at least tumor fraction estimation information associated with the plurality of variant calls (e.g., based at least in part on one or more respective called variants identified as somatic and/or germline for one or more corresponding allelic positions of the subject).

For example, in some embodiments, an untrained classifier was trained on a training set comprising one or more reference pluralities of variant calls (e.g., identified as somatic and/or germline), where each reference plurality of variant calls is associated with corresponding tumor fraction estimation information.

In some embodiments, the classifier was logistic regression. In some embodiments, the classifier was a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

Classifiers for use in some embodiments are described in further detail in, e.g., U.S. patent application Ser. No. 17/119,606,” filed Dec. 11, 2020, and United States Patent Publication No. 2020-0385813 A1, entitled “Systems and Methods for Estimating Cell Source Fractions Using Methylation Information,” filed Dec. 18, 2019, each of which is hereby incorporated herein by reference in its entirety.

In some embodiments, the classifier was based on a neural network algorithm, a support vector machine algorithm, a decision tree algorithm, an unsupervised clustering algorithm, a supervised clustering algorithm, or a logistic regression algorithm, a mixture model, or a hidden Markov model. In some embodiments, the trained classifier is a multinomial classifier.

In some embodiments the classifier made use of the B score classifier described in United States Patent Publication No. US 2019-0287649 A1, entitled “Method and System for Selecting, Managing, and Analyzing Data of High Dimensionality,” filed Mar. 13, 2019, which is hereby incorporated by reference.

In some embodiments, the classifier made use of the M score classifier described in United States Patent Publication No. US 2019-0287652 A1, entitled “Methylation Fragment Anomaly Detection,” filed Mar. 13, 2019, which is hereby incorporated by reference.

In some embodiments, the classifier was a neural network or a convolutional neural network. See, U.S. Patent Application No. 62/679,746, entitled “Convolutional Neural Network Systems and Methods for Data Classification,” filed Jun. 1, 2018, which is hereby incorporated by reference, for its disclosure of convolutional neural networks that can be used for classifying methylation patterns in accordance with the present disclosure.

In some embodiments, the classifier was a support vector machine (SVM). When used for classification, SVMs separate a given set of binary labeled data with a hyper-plane that is maximally distant from the labeled data. For cases in which no linear separation is possible, SVMs can work in combination with the technique of “kernels”, which automatically realizes a non-linear mapping to a feature space. The hyper-plane found by the SVM in feature space corresponds to a non-linear decision boundary in the input space.

In some embodiments, the classifier was a decision tree. Tree-based methods partition the feature space into a set of rectangles, and then fit a model (like a constant) in each one. In some embodiments, the decision tree was random forest regression. One specific algorithm that can be used is a classification and regression tree (CART). Other specific decision tree algorithms include, but are not limited to, ID3, C4.5, MART, and Random Forests.

In some embodiments, the classifier was an unsupervised clustering model. In some embodiments, the classifier is a supervised clustering model. The clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. Clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” Once a method for measuring “similarity” or “dissimilarity” between points in a dataset has been selected, clustering requires a criterion function that measures the clustering quality of any partition of the data. Partitions of the data set that extremize the criterion function used to cluster the data. Particular exemplary clustering techniques that can be used in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

In some embodiments, the classifier was a regression model, such as the multi-category logit models. In some embodiments, the classifier makes use of a regression model.

In some embodiments, the classifier was a Naive Bayes algorithm. In some embodiments, the classifier was a nearest neighbor algorithm, such as a non-parametric methods. In some embodiments, the classifier is a mixture model. In some embodiments, in particular, those embodiments including a temporal component, the classifier was a hidden Markov model.

In some embodiments, the classifier was an A score classifier. The A score classifier was a classifier of tumor mutational burden based on targeted sequencing analysis of nonsynonymous mutations. For example, a classification score (e.g., “A score”) can be computed using logistic regression on tumor mutational burden data, where an estimate of tumor mutational burden for each individual is obtained from the targeted cfDNA assay. In some embodiments, a tumor mutational burden can be estimated as the total number of variants per individual that are: called as candidate variants in the cfDNA, passed noise-modeling and joint-calling, and/or found as nonsynonymous in any gene annotation overlapping the variants. The tumor mutational burden numbers of a training set can be fed into a penalized logistic regression classifier to determine cutoffs at which 95% specificity is achieved using cross-validation.

In some embodiments, the classifier was a B score classifier. The B score classifier is described in United States Patent Publication No. US 2019-0287649 A1, entitled “Method and System for Selecting, Managing, and Analyzing Data of High Dimensionality,” which is hereby incorporated by reference. In accordance with the B score method, a first set of sequence reads of nucleic acid samples from healthy subjects in a reference group of healthy subjects are analyzed for regions of low variability. Accordingly, each sequence read in the first set of sequence reads of nucleic acid samples from each healthy subject is aligned to a region in the reference genome. From this, a training set of sequence reads from sequence reads of nucleic acid samples from subjects in a training group is selected. Each sequence read in the training set aligns to a region in the regions of low variability in the reference genome identified from the reference set. The training set includes sequence reads of nucleic acid samples from healthy subjects as well as sequence reads of nucleic acid samples from diseased subjects who are known to have the cancer. The nucleic acid samples from the training group are of a type that is the same as or similar to that of the nucleic acid samples from the reference group of healthy subjects. From this it is determined, using quantities derived from sequence reads of the training set, one or more metrics that reflect differences between sequence reads of nucleic acid samples from the healthy subjects and sequence reads of nucleic acid samples from the diseased subjects within the training group. Then, a test set of sequence reads associated with nucleic acid samples comprising cell-free nucleic acid fragments from a test subject whose status with respect to the cancer is unknown is received, and the likelihood of the test subject having the cancer is determined based on the one or more metrics.

In some embodiments, the classifier was an M score classifier. The M score classifier is described in United States Patent Publication No. US 2019-0287652 A1, entitled “Anomalous Fragment Detection and Classification,” which is hereby incorporated by reference.

### Example 4—Whole Genome Bisulfite Sequencing (WGBS)

WGBS is described in United States Patent Application Publication No. US 2019-0287652 A1 entitled “Anomalous Fragment Detection and Classification,” which is hereby incorporated by reference.

### Example 5—Cell-Free Genome Atlas Study (CCGA) Cohorts

Subjects from the CCGA [NCT02889978] were used in the Examples of the present disclosure. CCGA is a prospective, multi-center, observational cfDNA-based early cancer detection study that has enrolled 15,254 demographically balanced participants at 141 sites. Blood samples were collected from the 15,254 enrolled participants (56% cancer, 44% non-cancer) from subjects with newly diagnosed therapy-naive cancer (C, case) and participants without a diagnosis of cancer (noncancer [NC], control) as defined at enrollment.

In a first cohort (pre-specified substudy) (CCGA-1), plasma cfDNA extractions were obtained from 3,583 CCGA and STRIVE participants (CCGA: 1,530 cancer subjects and 884 non-cancer subjects; STRIVE 1,169 non-cancer participants). STRIVE is a multi-center, prospective, cohort study enrolling women undergoing screening mammography (99,259 participants enrolled). Blood was collected (n=1,785) from 984 CCGA participants with newly diagnosed, untreated cancer (20 tumor types, all stages) and 749 participants with no cancer diagnosis (controls) for plasma cfDNA extraction. This preplanned substudy included 878 cases, 580 controls, and 169 assay controls (n=1627) across twenty tumor types and all clinical stages.

Three sequencing assays were performed on the blood drawn from each participant: 1) paired cfDNA and white blood cell (WBC)-targeted sequencing (60,000×, 507 gene panel) for single nucleotide variants/indels (the ART sequencing assay); a joint caller removed WBC-derived somatic variants and residual technical noise; 2) paired cfDNA and WBC whole-genome sequencing (WGS; 35×) for copy number variation; a novel machine learning algorithm generated cancer-related signal scores; joint analysis identified shared events; and 3) cfDNA whole-genome bisulfite sequencing (WGBS; 34×) for methylation; normalized scores were generated using abnormally methylated fragments. In addition, tissue samples were obtained from participants with cancer, such that 4) whole-genome sequencing (WGS; 30X) was performed on paired tumor and WBC gDNA for identification of tumor variants for comparison.

Within the context of the CCGA-1 study, several methods were developed for estimating tumor fraction of a cfDNA sample. See, International Patent Publication No. WO/2019/204360, entitled “SYSTEMS AND METHODS FOR DETERMINING TUMOR FRACTION IN CELL-FREE NUCLEIC ACID,” International Patent Publication No. WO 2020/132148, entitled “SYSTEMS AND METHODS FOR ESTIMATING CELL SOURCE FRACTIONS USING METHYLATION INFORMATION,” and United States Patent Publication No. US 2020-0340064 A1, entitled “SYSTEMS AND METHODS FOR TUMOR FRACTION ESTIMATION FROM SMALL VARIANTS,” each of which is hereby incorporated by reference.

In a second pre-specified substudy (CCGA-2), a targeted, rather than whole-genome, bisulfite sequencing assay was used to develop a classifier of cancer versus non-cancer and tissue-of-origin based on a targeted methylation sequencing approach. For CCGA-2, 3,133 training participants and 1,354 validation samples (775 having cancer; 579 not having cancer as determined at enrollment, prior to confirmation of cancer versus non-cancer status) were used. Plasma cfDNA was subjected to a bisulfite sequencing assay (the COMPASS assay) targeting the most informative regions of the methylome, as identified from a unique methylation database and prior prototype whole-genome and targeted sequencing assays, to identify cancer and tissue-defining methylation signal. Of the original 3,133 samples reserved for training, 1,308 samples were deemed clinically evaluable and analyzable. Analysis was performed on a primary analysis population n=927 (654 cancer and 273 non-cancer) and a secondary analysis population n=1,027 (659 cancer and 373 non-cancer). Finally, genomic DNA from formalin-fixed, paraffin-embedded (FFPE) tumor tissues and isolated cells from tumors was subjected to whole-genome bisulfite sequencing (WGBS) to generate a large database of cancer-defining methylation signals for use in panel design and in training to optimize performance.

These data demonstrate the feasibility of achieving >99% specificity for invasive cancer and support the promise of cfDNA assay for early cancer detection. See, e.g., Klein et al., 2018, “Development of a comprehensive cell-free DNA (cfDNA) assay for early detection of multiple tumor types: The Circulating Cell-free Genome Atlas (CCGA) study,” J. Clin. Oncology 36(15), 12021-12021; doi: 10.1200/JCO.2018.36.15_supp1.12021, and Liu et al., 2019, “Genome-wide cell-free DNA (cfDNA) methylation signatures and effect on tissue of origin (TOO) performance,” J. Clin. Oncology 37(15), 3049-3049; doi: 10.1200/JCO.2019.37.15 supp1.3049, each of which is hereby incorporated herein by reference in its entirety.

Within the context of the CCGA-2 study, multiple methods were developed for estimating tumor fraction of a cfDNA sample based on methylation data (obtained by targeted methylation or WGBS) (see, e.g., International Patent Publication No. WO 2020/132148, entitled “SYSTEMS AND METHODS FOR ESTIMATING CELL SOURCE FRACTIONS USING METHYLATION INFORMATION,” and U.S. Provisional Patent Application No. 62/983,443 entitled “Identifying Methylation Patterns that Discriminate or Indicate a Cancer Condition,” filed Feb. 28, 2020, each of which is hereby incorporated by reference in its entirety). In an example approach, nucleic acid samples from formalin-fixed, paraffin-embedded (FFPE) tumor tissues were analyzed by whole-genome bisulfite sequencing (WGBS). Somatic variants identified based on the sequencing data were analyzed against matching cfDNA WGBS sequencing data from the same patient and used to determine a tumor fraction estimate.

### Example 6—Co-Occurrence of Abnormal Methylation Patterns with Somatic Variants

Experiment 1. An initial experiment was performed to determine whether a correlation exists between hyper-methylation and mutant fragments, via a simulated pull-down of hypermethylated fragments performed using Fisher's exact test and assessment for enrichment of somatic variants.

A dataset of 220 tissue samples sequenced using WGBS was subsetted to select for regions enriched for methylation. The dataset further included 13,500 somatic variants annotated using patient-matched tissue sequenced using WGS. The somatic variants were called based on an analysis including patient-matched normal tissue reference and were therefore considered ground truth. For each somatic variant, the dataset was split between “reference” or “alternate” fragments, based on whether each fragment in the dataset corresponding to the variant position supported the reference or alternate alleles. Each fragment was further determined to be hypo-methylated or hyper-methylated by calculating the methylation fraction (Beta-value) of each fragment. For instance, fragments with Beta-values greater than 0.5 were determined to be hyper-methylated, while fragments with Beta-values less than or equal to 0.5 were determined to be hypo-methylated. For each somatic variant, correlations between hyper-methylation and mutant fragments were assessed using Fisher's exact test, according to the matrix illustrated below.

| | Reference Allele | Alternate Allele |
|---|---|---|
| Methylated | # Fragments | # Fragments |
| Unmethylated | # Fragments | # Fragments |

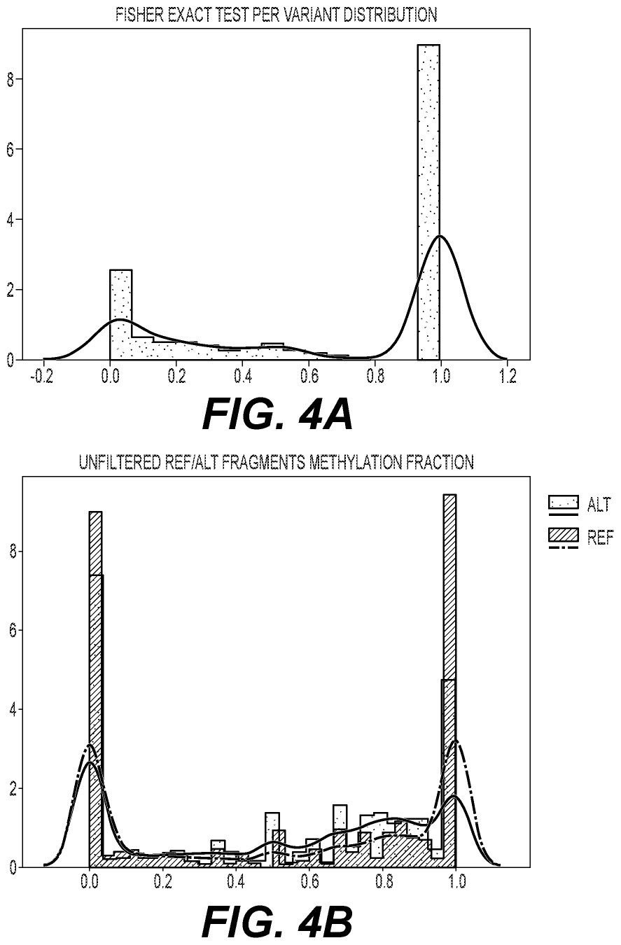

Hyper-methylated variants and hypo-methylated variants were aggregated and plotted, respectively. 6.6% of variants were found to be significantly associated with hyper-methylated (FDR <0.05), indicating that hypermethylated fragments did not significantly enrich for somatic variants in isolation. FIG. 4A illustrates these results using a distribution plot of the probability density of alternate fragments plotted against fragment Beta-values (x-axis) across variants.

An alternate approach was utilized to determine whether fragment-level, rather than variant-level, methylation fractions could be correlated with somatic variants. All fragments in the dataset were aggregated across variants together, faceting on reference and alternate support. The methylation fraction (Beta-value) was calculated for each fragment. FIG. 4B illustrates the distribution plot of the probability density of alternate fragments and reference fragments plotted against Beta-values (x-axis), further illustrating that alternate fragments were not significantly enriched at high methylation fractions.

Experiment 2. An experiment was performed to determine whether tumor-derived fragments as marked by methylation could be informative for somatic variant detection, particularly in the presence of nearby CpG sites.

A dataset of 238 tissue samples sequenced using WGBS from the CCGA-1 substudy (see, Example 5) was subsetted to select for regions enriched for methylation. A simplified variant calling workflow was performed using a Bayesian likelihood filter, the Single Nucleotide Polymorphism Database (dbSNP; NCBI), and a tissue recurrence blacklist, as disclosed in U.S. patent application Ser. No. 17/185,885, filed Feb. 25, 2021, entitled “Systems and Methods for Calling Variants using Methylation Sequencing Data,” and PCT Application No. PCT/US2021/019746, filed February 2021, entitled “Systems and Methods for Calling Variants using Methylation Sequencing Data,” each of which is hereby incorporated herein by reference in its entirety. The dataset included 12,928 somatic variants and 49,083 germline variants obtained using patient-matched tissue sequenced using WGS. For each candidate variant, fragments were grouped into “reference” or “alternate” bins based on whether each fragment supported either the reference allele or the alternate allele. For each candidate variant, p-value distribution statistics (e.g., mean, min, max, median, and standard deviation) across the reference bin and the alternate bin, respectively, were calculated. Additionally, for each candidate variant, distribution statistics for the number of CpG sites (e.g., mean, min, max, median, and standard deviation) across all fragments in the reference bin and the alternate bin, respectively, were calculated. Reference and alternate counts, p-values, numbers of CpG sites, and the distribution statistics thereof, were determined in accordance with some embodiments of the present disclosure, as disclosed herein. For each candidate variant, the obtained features (e.g., reference and alternate fragment counts, p-values, and/or CpG sites) were binned together into a fixed-length vector for the respective variant and used as input for training and evaluating a classifier for determining whether a candidate variant is somatic or germline. Classifiers were trained and evaluated using an 80/20 train-test variant split.

FIGS. 5A and 5B illustrate the performance of a baseline binary classification model using the reference and alternate fragment counts as inputs. FIG. 5A is a receiver operating characteristic (ROC) curve showing the evaluation of the performance of a logistic regression classifier for determining whether a candidate variant is somatic or germline. Similar performances were observed for both the training and testing datasets (training: AUC=0.70; testing: AUC=0.69). FIG. 5B illustrates a precision-recall curve for the logistic regression classifier, in which a 20% sensitivity (recall) is achieved at a 50% positive predictive value (PPV or precision). As defined above, the positive predictive value (PPV) refers to the proportion of variants that are correctly categorized as a somatic or germline variant (e.g., the number of true positives divided by the sum of the number of true positives and the number of false positives).

In contrast, FIGS. 6A and 6B illustrate the performance of a binary classification model using an expanded feature input, including reference and alternate fragment counts, p-value distribution statistics (e.g., mean, min, max, median, and standard deviation), and distribution statistics for the number of CpG sites (e.g., mean, min, max, median, and standard deviation) across all fragments for each of the reference bin and the alternate bin, respectively. FIG. 6A is a ROC curve showing the evaluation of the performance of a multi-layer perceptron (MLP) neural network classifier for determining whether a candidate variant is somatic or germline. Similar performances were observed for both the training and testing datasets (training: AUC=0.80; testing: AUC=0.80) and is further improved compared to the previous model utilizing the reference and alternate fragment counts as input. In addition, FIG. 6B illustrates the precision-recall curve for the MLP classifier, in which the sensitivity (recall) achieved at a 50% positive predictive value (PPV or precision) is 60%, compared to 20% in the previous model.

Experiment 3. An additional experiment was performed to determine whether tumor-derived fragments as marked by methylation could be informative for somatic variant detection in cfDNA samples. A dataset of 148 cfDNA samples sequenced using targeted methylation was subsetted to select for regions enriched for methylation. The dataset included 404 somatic variants and 62,575 germline variants annotated using WGS and filtered to remove variants with zero read support in fragments sequenced from the cfDNA samples (e.g., filter for variants having a non-zero alternate support depth). Classifiers were trained and evaluated using an 80/20 train-test variant split.

FIGS. 10A and 10B illustrate the performance of a baseline binary classification model using the reference and alternate fragment counts as inputs. FIG. 10A is the ROC curve showing the evaluation of the performance of a logistic regression classifier for determining whether a candidate variant is somatic or germline. Similar performances were observed for both the training and testing datasets (training: AUC=0.63; testing: AUC=0.63). FIG. 10B illustrates the precision-recall curve for the logistic regression classifier, showing that variants are poorly resolved as indicated by the low precision obtained by the model (likely due to low tumor signal and a high proportion of noise from normal-derived fragments in the cfDNA samples compared to tissue samples).

In contrast, FIGS. 11A and 11B illustrate the performance of the model using the expanded feature input, including reference and alternate fragment counts, p-value distribution statistics (e.g., mean, min, max, median, and standard deviation), and distribution statistics for the number of CpG sites (e.g., mean, min, max, median, and standard deviation) across all fragments for each of the reference bin and the alternate bin, respectively. FIG. 11A is the ROC curve showing the evaluation of the performance of the logistic regression model, in which similar performances were observed for both the training and testing datasets (training: AUC=0.86; testing: AUC=0.85) and reveal an improvement over the model utilizing the reference and alternate fragment counts as input (training: AUC=0.63; testing: AUC=0.63). In addition, FIG. 11B illustrates the precision-recall curve for the logistic regression model, showing improved PPV, with approximately 30% sensitivity achieved at approximately 10% PPV.

Conclusions. The data indicate that abnormal methylation patterns co-occur with somatic variants given CpG sites are present within the vicinity of a variant. For example, in WGBS tissue, this relationship can be used to achieve a similar PPV (50%) to filtering methods previously used within tumor fraction estimation methods with WGS cfDNA, albeit with a 40% loss in sensitivity. See, for example, U.S. patent application Ser. No. 17/185,885, filed Feb. 25, 2021, entitled “Systems and Methods for Calling Variants using Methylation Sequencing Data,” and PCT Application No. PCT/US2021/019746, filed February 2021, entitled “Systems and Methods for Calling Variants using Methylation Sequencing Data,” each of which is hereby incorporated herein by reference in its entirety.

In targeted methylation cfDNA, the above experiments revealed an increase in PPV for somatic variant detection when using expanded feature inputs. In some instances, larger training datasets and methods for reducing the class balance can be used to offset the differential between somatic and germline variants in cfDNA (e.g., more closely approximating class balances in tissue), which may further improve PPV and sensitivity.

### CONCLUSION

The terminology used herein is for the purpose of describing particular cases only and is not intended to be limiting. As used herein, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed:

1. A method of identifying a variant allele at a genomic position in a test subject as somatic or germline, the method comprising:
obtaining an identification of a reference allele at the genomic position;
obtaining an identification of the variant allele at the genomic position;
obtaining a methylation state and a respective sequence of each nucleic acid fragment sequence in a respective plurality of nucleic acid fragment sequences in a sequencing dataset derived from a liquid biological sample obtained from the test subject that map onto the genomic position, wherein the sequencing dataset comprises at least 1×10⁶ nucleic acid fragment sequences;
using (i) the identification of the reference allele at the genomic position and (ii) the respective sequence of each nucleic acid fragment sequence in the respective plurality of nucleic acid fragment sequences to assign each nucleic acid fragment sequence in the respective plurality of nucleic acid fragment sequences that has the reference allele, at the genomic position, to a reference subset;
using (i) the identification of the variant allele at the genomic position and (ii) the respective sequence of each nucleic acid fragment sequence in the respective plurality of nucleic acid fragment sequences to assign each nucleic acid fragment sequence in the respective plurality of nucleic acid fragment sequences that has the variant allele, at the genomic position, to a variant subset; and
applying, to a trained binary classifier, at least (i) one or more indications of methylation state across the methylation state of each nucleic acid fragment sequence in the variant subset and (ii) an indication of a number of nucleic acid fragment sequences in the reference subset versus a number of nucleic acid fragment sequences in the variant subset, wherein the trained binary classifier comprises at least 10 parameters, thereby obtaining from the trained binary classifier an identification of the variant allele at the genomic position in the test subject as somatic or germline.

2. The method of claim 1, wherein the method further comprises:
inputting a reference genome into a computer system comprising a processor coupled to a non-transitory memory, and
using the computer system to determine that each respective nucleic acid fragment sequence in the respective plurality of nucleic acid fragment sequences maps to the genomic position by aligning the respective nucleic acid fragment sequence to the reference genome.

3. The method of claim 1, wherein
a first nucleic acid fragment sequence in the respective plurality of nucleic acid fragment sequences has a plurality of CpG sites;
wherein the first nucleic acid fragment sequence has a corresponding methylation pattern across the plurality of CpG sites;
wherein the methylation state of the first nucleic acid fragment sequence is a p-value, and
wherein the method further comprises:
determining the p-value of the first nucleic acid fragment sequence, at least in part, by comparison of the corresponding methylation pattern of the first nucleic acid fragment sequence to a corresponding distribution of methylation patterns of those nucleic acid fragment sequences in a healthy noncancer cohort dataset that each have the respective plurality of CpG sites.

4. The method of claim 1, wherein the variant allele is an insertion, a deletion, or a single nucleotide polymorphism.

5. The method of claim 1, wherein, when the variant allele at the genomic position is determined by the trained binary classifier to be germline, the method further comprises:
using the variant allele in the test subject to perform an action selected from the group consisting of: determining a cancer risk of the test subject, predicting an ethnicity of the test subject, and determining a tumor fraction of the test subject.

6. The method of claim 1, wherein:
each indication in the one or more indications of methylation state across the variant subset is:
a measure of central tendency of a methylation state p-value across the variant subset,
a minimum methylation state p-value across the variant subset,
a maximum methylation state p-value across the variant subset, or
a measure of spread of a methylation state p-value across the variant subset.

7. The method of claim 1, wherein the one or more indications of methylation state across the variant subset is a plurality of indications of methylation state across the variant subset comprising at least 2, at least 3, or all four of:
a measure of central tendency of a methylation state p-value across the variant subset,
a minimum methylation state p-value across the variant subset,
a maximum methylation state p-value across the variant subset, and
a measure of spread of a methylation state p-value across the variant subset.

8. The method of claim 1, wherein the applying, to the trained binary classifier, further applies one of:
one or more CpG site indications across the variant subset;
one or more indications of methylation state across the reference subset; or
one or more CpG site indications across the reference subset.

9. The method of claim 1, wherein the obtaining an identification of the variant allele at the genomic position comprises determining that the respective plurality of nucleic acid fragments support a variant allele call at the genomic position.

10. The method of claim 1, further comprising performing methylation sequencing to obtain the methylation state and the respective sequence of each nucleic acid fragment sequence in the respective plurality of nucleic acid fragment sequences.

11. A computing system, comprising:
one or more processors;
memory storing one or more programs to be executed by the one or more processor, the one or more programs comprising instructions for calling a variant at a genomic position in a test subject by a method comprising:
obtaining an identification of a reference allele at the genomic position;
obtaining an identification of the variant allele at the genomic position;
obtaining a methylation state and a respective sequence of each nucleic acid fragment sequence in a respective plurality of nucleic acid fragment sequences in a sequencing dataset derived from a liquid biological sample obtained from the test subject that map onto the genomic position, wherein the sequencing dataset comprises at least 10\hat{ }6 nucleic acid fragment sequences;
using (i) the identification of the reference allele at the genomic position and (ii) the respective sequence of each nucleic acid fragment sequence in the respective plurality of nucleic acid fragment sequences to assign each nucleic acid fragment sequence in the respective plurality of nucleic acid fragment sequences that has the reference allele, at the genomic position, to a reference subset;
using (i) the identification of the variant allele at the genomic position and (ii) the respective sequence of each nucleic acid fragment sequence in the respective plurality of nucleic acid fragment sequences to assign each nucleic acid fragment sequence in the respective plurality of nucleic acid fragment sequences that has the variant allele, at the genomic position, to a variant subset; and
applying, to a trained binary classifier, at least (i) one or more indications of methylation state across the methylation state of each nucleic acid fragment sequence in the variant subset and (ii) an indication of a number of nucleic acid fragment sequences in the reference subset versus a number of nucleic acid fragment sequences in the variant subset, wherein the trained binary classifier comprises at least 10 parameters, thereby obtaining from the trained binary classifier an identification of the variant allele at the genomic position in the test subject as somatic or germline.

12. The computing system of claim 11, wherein the instructions further comprise:
inputting a reference genome into a computer system comprising a processor coupled to a non-transitory memory, and
using the computer system to determine that each respective nucleic acid fragment sequence in the respective plurality of nucleic acid fragment sequences maps to the genomic position by aligning the respective nucleic acid fragment sequence to the reference genome.

13. The computing system of claim 11, wherein:
a first nucleic acid fragment sequence in the respective plurality of nucleic acid fragment sequences has a plurality of CpG sites;
wherein the first nucleic acid fragment sequence has a corresponding methylation pattern across the plurality of CpG sites;
wherein the methylation state of the first nucleic acid fragment sequence is a p-value, and
wherein the instructions further comprise:
determining the p-value of the first nucleic acid fragment sequence, at least in part, by comparison of the corresponding methylation pattern of the first nucleic acid fragment sequence to a corresponding distribution of methylation patterns of those nucleic acid fragment sequences in a healthy noncancer cohort dataset that each have the respective plurality of CpG sites.

14. The computing system of claim 11, wherein, when the variant allele at the genomic position is determined by the trained binary classifier to be germline, the instructions further comprise:
using the variant allele in the test subject to perform an action selected from the group consisting of: determining a cancer risk of the test subject, predicting an ethnicity of the test subject, and determining a tumor fraction of the test subject.

15. The computing system of claim 11, wherein:
each indication in the one or more indications of methylation state across the variant subset is:
a measure of central tendency of a methylation state p-value across the variant subset,
a minimum methylation state p-value across the variant subset,
a maximum methylation state p-value across the variant subset, or
a measure of spread of a methylation state p-value across the variant subset.

16. The computing system of claim 11, wherein the one or more indications of methylation state across the variant subset is a plurality of indications of methylation state across the variant subset comprising at least 2, at least 3, or all four of:
a measure of central tendency of a methylation state p-value across the variant subset,
a minimum methylation state p-value across the variant subset,
a maximum methylation state p-value across the variant subset, and
a measure of spread of a methylation state p-value across the variant subset.

17. The computing system of claim 11, wherein the instruction to apply, to the trained binary classifier, further comprise instructions to apply one of:
one or more CpG site indications across the variant subset;
one or more indications of methylation state across the reference subset; or
one or more CpG site indications across the reference subset.

18. The computing system of claim 11, wherein the instructions to obtain an identification of the variant allele at the genomic position comprise instructions to determine that the respective plurality of nucleic acid fragments support a variant allele call at the genomic position.

19. The computing system of claim 11, wherein the instructions further comprise performing methylation sequencing to obtain the methylation state and the respective sequence of each nucleic acid fragment sequence in the respective plurality of nucleic acid fragment sequences.

20. A non-transitory computer readable storage medium storing one or more programs for calling a variant at a genomic position in a test subject, the one or more programs configured for execution by a computer, wherein the one or more programs comprise instructions for:
obtaining an identification of a reference allele at the genomic position;
obtaining an identification of the variant allele at the genomic position;
obtaining a methylation state and a respective sequence of each nucleic acid fragment sequence in a respective plurality of nucleic acid fragment sequences in a sequencing dataset derived from a liquid biological sample obtained from the test subject that map onto the genomic position, wherein the sequencing dataset comprises at least 10\hat{ }6 nucleic acid fragment sequences;
using (i) the identification of the reference allele at the genomic position and (ii) the respective sequence of each nucleic acid fragment sequence in the respective plurality of nucleic acid fragment sequences to assign each nucleic acid fragment sequence in the respective plurality of nucleic acid fragment sequences that has the reference allele, at the genomic position, to a reference subset;
using (i) the identification of the variant allele at the genomic position and (ii) the respective sequence of each nucleic acid fragment sequence in the respective plurality of nucleic acid fragment sequences to assign each nucleic acid fragment sequence in the respective plurality of nucleic acid fragment sequences that has the variant allele, at the genomic position, to a variant subset; and
applying, to a trained binary classifier, at least (i) one or more indications of methylation state across the methylation state of each nucleic acid fragment sequence in the variant subset and (ii) an indication of a number of nucleic acid fragment sequences in the reference subset versus a number of nucleic acid fragment sequences in the variant subset, wherein the trained binary classifier comprises at least 10 parameters, thereby obtaining from the trained binary classifier an identification of the variant allele at the genomic position in the test subject as somatic or germline.

21. A method of training a classifier to identify a variant allele at a genomic position in a test subject as somatic or germline, the method comprising:
A) obtaining an identification of a reference allele at the genomic position;
B) for each respective subject in a plurality of subjects, for each respective genomic position in a plurality of genomic positions, performing a procedure comprising:
i) obtaining an orthogonal call for the variant allele at the respective genomic position as one of somatic or germline for the respective subject;
ii) obtaining an identification of the variant allele at the respective genomic position for the respective subject;
iii) obtaining a methylation state and a respective sequence of each nucleic acid fragment sequence in a respective plurality of nucleic acid fragment sequences in a sequencing dataset derived from a liquid biological sample obtained from the respective subject that map onto the respective genomic position, wherein the sequencing dataset comprises at least 1×10⁶ nucleic acid fragment sequences;
iv) using (a) the identification of the reference allele at the respective genomic position and (b) the respective sequence of each nucleic acid fragment sequence in the respective plurality of nucleic acid fragment sequences to assign each nucleic acid fragment sequence in the respective plurality of nucleic acid fragment sequences that has the reference allele, at the respective genomic position, to a reference subset;
v) using (a) the identification of the variant allele at the respective genomic position and (b) the respective sequence of each nucleic acid fragment sequence in the respective plurality of nucleic acid fragment sequences to assign each nucleic acid fragment sequence in the respective plurality of nucleic acid fragment sequences that has the variant allele, at the respective genomic position, to a variant subset; and
C) using, for each respective subject in the plurality of subjects, for each respective genomic position in the plurality of genomic positions, at least (i) one or more indications of methylation state across the methylation state of each nucleic acid fragment sequence in the variant subset for the respective subject for the respective genomic position (ii) an indication of a number of nucleic acid fragment sequences in the reference subset versus a number of nucleic acid fragment sequences in the variant subset for the respective subject for the respective genomic position and (iii) the orthogonal call for the variant allele at the respective genomic position as one of somatic or germline for the respective subject to train the classifier to identify a variant allele at a genomic position in a test subject as somatic or germline, wherein the classifier comprises at least 10 parameters.
